(19) United States
(12) Patent Application Publication
(10) Pub. No.: US 2019/0099291 A1
(43) Pub. Date: Apr. 4, 2019

(54) EFFECTIVE OCULAR LENS POSITIONING METHODS AND APPARATUS

(71) Applicant: ALEYEGN TECHNOLOGIES LLC, Saratoga, CA (US)

(72) Inventors: Rajeev HEREKAR, PALO ALTO, CA (US); Satish V. HEREKAR, PALO ALTO, CA (US)

(73) Assignee: ALEYEGN TECHNOLOGIES LLC, Saratoga, CA (US)

(21) Appl. No.: 16/134,334

(22) Filed: Sep. 18, 2018

Related U.S. Application Data
(63) Continuation of application No. PCT/US2017/023092, filed on Mar. 17, 2017.
(60) Provisional application No. 62/310,638, filed on Mar. 18, 2016.

(30) Foreign Application Priority Data
Mar. 17, 2017 (US) PCT/US2017/023092

(51) Int. Cl. A61F 9/007; A61B 18/12; A61N 7/02

(52) U.S. Cl. CPC A61F 9/007; A61F 2009/0087; A61N 7/02; A61B 18/12

(57) ABSTRACT

The methods and apparatus disclosed herein can be used to adjust the effective lens position (“ELP”) of the eye in order to correct refractive error of the eye. The methods and apparatus can be configured to apply energy to the sclera and other regions of the eye in order to adjust the effective lens position of the eye for far vision. The sclera can be treated in order to shrink or relax the sclera, and combinations thereof in order to adjust the position of the lens of the eye in order to correct vision related to refractive error of the eye. A target location of the lens can be determined to correct the refractive error of the eye, and the energy applied in order to move the lens toward the target location.

### CROSS-REFERENCE TO RELATED APPLICATIONS

The present application is a continuation of International Patent Application No. PCT/US2017/023092, filed Mar. 17, 2017, entitled “EFFECTIVE OCULAR LENS POSITIONING METHODS AND APPARATUS”, which claims priority to U.S. Provisional Application No. 62/310,638, filed Mar. 18, 2016, entitled “EFFECTIVE OCULAR LENS POSITIONING METHODS AND APPARATUS”, the entire disclosure of each of which is incorporated herein by reference.

The subject matter of the present application is related to PCT Application No. PCT/US2016/055829, filed on Oct. 6, 2016, entitled “ULTRASOUND DIRECTED CAVITATIONAL METHODS AND SYSTEM FOR OCULAR TREATMENTS” (attorney docket no. 48848-704.601), the entire disclosure of which is incorporated herein by reference.

### BACKGROUND OF THE INVENTION

Vision correction of the eye such as for the treatment of refractive error and correction of presbyopia have proven challenging. Prior methods and apparatus can be less than ideal in at least some respects. For example, many of the prior approaches can be more complex and/or invasive than would be ideal. For example, prior approaches such as LASIK can result in surgical removal and replacement of a portion of the cornea. Intraocular lenses, while effective, usually require a surgical incision and may require removal of the lens of the eye. Also, intraocular lenses can leave patients with residual amounts of refractive error such that vision correction with glasses may be appropriate after surgery. Although infrared lasers have been used to treat the cornea and alter the curvature, such approaches can result in regression such that the effect of treatment decreases over time.

### SUMMARY OF THE INVENTION

The methods and apparatus disclosed herein can be used to adjust the effective lens position (“ELP”) of the eye in order to correct refractive error of the eye. The methods and apparatus can be configured to apply energy to the sclera and other regions of the eye in order to adjust the effective lens position of the eye for far vision. The sclera can be treated in order to shrink or relax the sclera, and combinations thereof in order to adjust the position of the lens of the eye in order to correct vision related to refractive error of the eye. The shrinkage of the sclera or corneal tissue can be located so as to move the ciliary body, which is connected to the lens with zonules and can result in movement of the lens. The relaxation of tissue can facilitate lens movement by allowing the lens to move to the desired location. In some embodiments, the tissue shrinkage in combination with relaxed tissue allows for more effective placement of the lens. A treatment pattern can be determined that allows the lens to move to a position that corrects vision. The treatment pattern may comprise a pattern of a nomogram that specifies amounts and locations of energy treatment to move the eye into position to correct a specified amount of refractive error, for example one Diopter. Alternatively or in combination, a target location of the lens can be determined to correct the refractive error of the eye, and the energy applied in order to move the lens toward the target location. Although reference is made to generally annular energy treatment patterns, the energy treatment pattern can be delivered in many ways, and may comprise a portion of an annulus, a polygon, or interspersed treatment along a generally circumferential pattern along the treatment region of the eye.

The energy can be applied to the eye in many ways, for example with one or more of laser energy, ultrasound energy or radiofrequency (RF) energy. The energy can be delivered with an optical or ultrasound delivery system, and the system may comprise an energy delivery system to direct energy to the eye or a hand held probe.

In one aspect, an apparatus to adjust one or more of an effective position or an orientation of a lens of the eye is provided. The apparatus comprises: an energy source configured to deliver energy to the eye to one or more of shrink or relax a sclera of the eye in order to adjust the one or more of the effective position or orientation of the lens of the eye.

In some embodiments, the energy source is configured to move the lens anteriorly to treat farsightedness, to move the lens posteriorly to treat nearsightedness, or to tilt the lens to treat astigmatism, and optionally combinations thereof.

In some embodiments, the apparatus may further comprise a processor configured with instructions to determine the effective position or orientation of the lens of the eye and to determine a direction of movement or rotation of the lens toward the effective lens position or orientation and optionally wherein the direction of movement or rotation is selected from the group consisting of posterior movement to treat myopia, anterior movement to treat hyperopia and rotation to treat astigmatism. Optionally, the processor may be coupled to the energy source and configured to heat sclera of the eye to a temperature within a range from about 50 C to about 70 C for an amount of time sufficient to shrink the sclera of the eye in order to move the lens of the eye toward the effective lens position and optionally wherein the processor is configured with instructions to heat the sclera of the eye to a temperature within a range from about 50 C to about 80 C for an amount of time sufficient to relax the sclera of the eye in order to move the lens toward the effective lens position. In some instances, the processor may be configured to determine a scanning pattern of energy to be delivered to the eye to move the lens toward the effective lens position. Alternatively or in addition, the processor may be coupled to the energy source and configured to deliver shrinkage energy to a first region of the sclera to shrink the sclera and relaxation energy to a second region of the sclera to relax the sclera in response to the direction of movement or rotation of the lens. In some embodiments, a target lens position may correspond to a target plane of the lens, and the shrinkage region and the relaxation region may be arranged on opposite sides of the target plane. Optionally, the relaxation region of the sclera may correspond to a location of an equator of the lens prior to treatment and the lens moves away from the relaxation region toward the stiffened region with treatment. Alternatively, a target orientation of the lens may correspond to a target plane of the lens, and the shrinkage region and the relaxation region may be arranged on the same side of the target plane and on opposite sides of the eye in order to urge the lens toward the target orientation. In some embodiments, the lens rotates away from the relaxation region on a first side of the eye and toward the shrinkage region on the second side of the eye. In some embodiments, the processor is configured to receive a refraction of the eye and to determine an amount of movement of the lens of the eye to adjust the refraction of the eye to a target refraction of the eye and wherein movement of the lens anteriorly adjusts the refraction of the eye negatively and movement of lens posteriorly adjusts the refraction of the eye positively. The processor may in some instances be configured to determine a scanning pattern of energy to be delivered to the eye to move the lens to adjust the refraction to the target amount. Alternatively or in addition, the processor is configured to adjust the refraction of the lens at a rate within a range from about 0.75 D to about 1.75 D per mm of movement of the lens. Optionally, the processor is configured to receive an axial length of the eye to determine the amount of movement.

In some embodiments, a target lens position may correspond to a target plane of the lens, and wherein the shrinkage region and the relaxation region are arranged on opposite sides of the target plane to move the lens away from the relaxation region and toward the shrinkage region.

In some embodiments, the apparatus may further comprise a delivery system configured to deliver the energy wherein the delivery system is configured to deliver the energy to one or more of a relaxation region of the sclera or a shrinkage region of the sclera, in order to move the lens anteriorly to treat farsightedness, posteriorly to treat nearsightedness, or to tilt the lens to treat astigmatism, and optionally combinations thereof. The delivery system may in some instances be configured to deliver an annular pattern of shrinkage energy anterior to the lens equator in order to move the lens anteriorly. Alternatively or in addition, the delivery system may be configured to deliver an annular pattern of shrinkage energy posterior to the lens equator in order to move the lens posteriorly. In some embodiments, the delivery system may be configured to deliver an annular pattern of relaxation energy anterior to the lens equator in order to move the lens posteriorly. Optionally, the delivery system may be configured to deliver an annular pattern of relaxation energy posterior to the lens equator in order to move the lens anteriorly.

In some embodiments, one or more of the energy source, the processor or the energy delivery system may be configured to deliver energy to a scleral shrinkage region posterior to the lens equator in order to urge the lens posteriorly to treat myopia. Alternatively or in addition, one or more of the energy source, the processor or the energy delivery system is configured to deliver energy to a scleral shrinkage region anterior to the lens equator in order to urge the lens anteriorly to treat hyperopia. In some embodiments, one or more of the energy source, the processor or the energy delivery system may be configured to deliver energy to a scleral shrinkage region anterior to the lens equator on a side of the eye in order to tilt the lens with anterior movement of a portion of the lens on the side of the eye. Similarly, one or more of the energy source, the processor or the energy delivery system may be configured to deliver energy to a scleral shrinkage region posterior to the lens equator on a side of the eye in order to tilt the lens with posterior movement of a portion of the lens on the side of the eye. Alternatively or in addition, one or more of the energy source, the processor or the energy delivery system may be configured to deliver energy to a scleral relaxation region anterior to the lens equator in order to urge the lens posteriorly to treat myopia. Optionally, one or more of the energy source, the processor or the energy delivery system may be configured to deliver energy to a scleral relaxation region posterior to the lens equator in order to urge the lens anteriorly to treat hyperopia. In some embodiments, one or more of the energy source, the processor or the energy delivery system may be configured to deliver energy to a scleral relaxation region posterior to the lens equator on a side of the eye in order to tilt the lens with anterior movement of a portion of the lens on the side of the eye. In other instances, one or more of the energy source, the processor or the energy delivery system may be configured to deliver energy to a scleral relaxation region anterior to the lens equator on a side of the eye in order to tilt the lens with posterior movement of a portion of the lens on the side of the eye.

In some embodiments, a target lens position corresponds to a target plane of the lens, and wherein the shrinkage region and the relaxation region are arranged on opposite sides of the target plane to move the lens away from the relaxation region and toward the shrinkage region.

In another aspect, a method of adjusting one or more of an effective position or an orientation a lens of the eye is provided. The method comprises: delivering energy to the eye to one or more of shrink or relax a sclera of the eye in order to adjust the one or more of the effective position or orientation of the lens of the eye.

In some embodiments, the energy source moves the lens anteriorly to treat farsightedness, posteriorly to treat nearsightedness, or tilts the lens to treat astigmatism, and optionally combinations thereof.

In some embodiments, the effective position or orientation of the lens of the eye is determined and a direction of movement or rotation of the lens toward the effective position or orientation is determined and wherein shrinkage energy is delivered to a first region of the sclera to shrink the sclera and relaxation energy is delivered to a second region of the sclera to relax the sclera in response to the direction of movement or rotation of the lens.

In some embodiments, a target lens position corresponds to a target plane of the lens, and wherein the shrinkage region and the relaxation region are arranged on opposite sides of the target plane to urge the lens toward the target position. In some instances, the relaxation region of the sclera may correspond to a location of an equator of the lens prior to treatment and the lens may move away from the relaxation region toward the stiffened region with treatment.

In some embodiments, a target orientation of the lens corresponds to a target plane of the lens, and wherein the shrinkage region and the relaxation region are arranged on the same side of the target plane and on opposite sides of the eye in order to urge the lens toward the target orientation. In some instances, the lens rotates away from the relaxation region on a first side of the eye and toward the shrinkage region on the second side of the eye.

In some embodiments, a delivery system delivers the energy to one or more of a relaxation region of the sclera or a shrinkage region of the sclera, in order to move the lens anteriorly to treat farsightedness, posteriorly to treat nearsightedness, or to tilt the lens to treat astigmatism, and optionally combinations thereof. The delivery system is configured to deliver an annular pattern of shrinkage energy anterior to the lens equator in order to move the lens anteriorly. Alternatively or in addition, the delivery system may be configured to deliver an annular pattern of shrinkage energy posterior to the lens equator in order to move the lens posteriorly. In certain instances, the delivery system may be configured to deliver an annular pattern of relaxation energy anterior to the lens equator in order to move the lens posteriorly. Optionally, the delivery system may be configured to deliver an annular pattern of relaxation energy posterior to the lens equator in order to move the lens anteriorly.

In some embodiments, energy may be delivered to a scleral shrinkage region posterior to the lens equator in order to urge the lens posteriorly to treat myopia. Alternatively or in addition, energy may be delivered to a scleral shrinkage region anterior to the lens equator in order to urge the lens anteriorly to treat hyperopia. Optionally, energy may be delivered to a scleral shrinkage region anterior to the lens equator on a side of the eye in order to tilt the lens with anterior movement of a portion of the lens on the side of the eye. In certain embodiments, energy may be delivered to a scleral shrinkage region posterior to the lens equator on a side of the eye in order to tilt the lens with posterior movement of a portion of the lens on the side of the eye. Energy, alternatively or in addition, may also be delivered to a scleral relaxation region anterior to the lens equator in order to urge the lens posteriorly to treat myopia. In some embodiments, energy may be delivered to a scleral relaxation region posterior to the lens equator in order to urge the lens anteriorly to treat hyperopia. Similarly, energy may be delivered to a scleral relaxation region posterior to the lens equator on a side of the eye in order to tilt the lens with anterior movement of a portion of the lens on the side of the eye. In some embodiments, energy may be delivered to a scleral relaxation region anterior to the lens equator on a side of the eye in order to tilt the lens with posterior movement of a portion of the lens on the side of the eye.

In some embodiments, the energy may comprise one or more of thermal energy, mechanical energy, electrical energy, light energy, ultrasound energy, and high intensity ultrasound energy.

In some embodiments, the energy comprises light energy comprising a wavelength within a range from about 1.3 to 1.6 um or from about 1.9 to 2.1 um.

In some embodiments, the processor is configured with instructions to deliver an amount of energy to a treatment region of the eye in order to move the lens to correct refractive error of the eye, in which the amount of energy is within a range from about 50 Joules to about 300 Joules per Diopter of change in optical power of the eye.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a presbyopic eye in a configuration for far vision, in accordance with embodiments;

FIG. 1A shows zonules of the eye and a treatment zone to adjust the effective lens position, in accordance with embodiments;

FIG. 2 illustrates the presbyopic eye of FIG. 1 attempting to correct for near vision, in accordance with embodiments;

FIG. 3 illustrates stabilization of an eye by cross-linking to treat presbyopia, in accordance with embodiments;

FIG. 4 illustrates a heat sink placed over the eye of FIG. 3 to treat presbyopia, in accordance with embodiments;

FIG. 5 illustrates a planned treatment zone to expand the circumlental space in the eye of FIG. 4 to treat presbyopia, in accordance with embodiments;

FIG. 6 illustrates ELP adjustment treatment of the eye of FIG. 5 to treat presbyopia, in accordance with embodiments;

FIG. 7 illustrates the eye of FIG. 6 in a configuration for near vision, in accordance with embodiments;

FIG. 8 illustrates the eye of FIG. 7 in a configuration for far vision, in accordance with embodiments;

FIG. 9 illustrates ELP adjustment softening of the insertion location of the posterior vitreal zonules of the eye of FIG. 8 to treat presbyopia, in accordance with embodiments;

FIG. 10 illustrates a planned treatment to enhance corneal bending of the eye of FIG. 9 to treat presbyopia, in accordance with embodiments;

FIG. 11 illustrates an ELP adjustment system for treating an eye, in accordance with embodiments;

FIG. 12 shows a treatment system for ELP adjustment, in accordance with embodiments;

FIG. 13 shows an ELP adjustment system, in accordance with embodiments;

FIG. 14 shows a HIFU array coupled to an imaging apparatus, in accordance with embodiments;

FIG. 15 shows another HIFU array coupled to an imaging apparatus, in accordance with embodiments;

FIG. 16 shows an effective lens position (ELP) adjustment, in accordance with embodiments;

FIG. 17 shows a lens with a normal ELP, in accordance with embodiments;

FIG. 18 shows another treatment zone for ELP adjustment, in accordance with embodiments;

FIG. 19 shows another treatment zone for ELP adjustment, in accordance with embodiments;

FIG. 20 shows yet another treatment zone for ELP adjustment, in accordance with embodiments;

FIG. 21 shows an additional treatment zone for ELP adjustment, in accordance with embodiments;

FIG. 22 shows a simulation of a model eye without axial displacement of the lens, in accordance with embodiments;

FIG. 23 shows a simulated image of a boy on the retina of the eye of FIG. 22, in accordance with embodiments;

FIG. 24 shows the root mean square (RMS) values for best focal position onto the retina of the eye in FIG. 22, in accordance with embodiments;

FIG. 25 shows a simulation of a model eye with +1.0 mm axial displacement of the lens, in accordance with embodiments;

FIG. 26 shows a simulated image of a boy on the retina of the eye of FIG. 25, in accordance with embodiments;

FIG. 27 shows the RMS values for best focal position onto the retina of the eye in FIG. 25, in accordance with embodiments;

FIG. 28 shows a simulation of a model eye with −1.0 mm axial displacement of the lens, in accordance with embodiments;

FIG. 29 shows a simulated image of a boy on the retina of the eye of FIG. 28, in accordance with embodiments;

FIG. 30 shows the RMS values for best focal position onto the retina of the eye in FIG. 28, in accordance with embodiments;

FIG. 31 shows a simulation of a model eye without lens tilt with respect to the central axis of the lens, in accordance with embodiments;

FIG. 32 shows a simulated image of a boy on the retina of the eye of FIG. 31, in accordance with embodiments;

FIG. 33 shows a spot diagram of different axial positions before and after the retina of the eye of FIG. 31, in accordance with embodiments;

FIG. 34 shows the RMS values for best focal position onto the retina of the eye in FIG. 31, in accordance with embodiments;

FIG. 35 shows a Ray Fan diagram for the eye of FIG. 31, in accordance with embodiments;

FIG. 36 shows a simulation of a model eye with 5° lens tilt with respect to the central axis of the lens, in accordance with embodiments;

FIG. 37 shows a simulated image of a boy on the retina of the eye of FIG. 36, in accordance with embodiments;

FIG. 38 shows a spot diagram of different axial positions before and after the retina of the eye of FIG. 36, in accordance with embodiments;

FIG. 39 shows the RMS values for best focal position onto the retina of the eye in FIG. 36, in accordance with embodiments;

FIG. 40 shows a Ray Fan diagram for the eye of FIG. 36, in accordance with embodiments;

FIG. 41 shows a simulation of a model eye with 10° lens tilt with respect to the central axis of the lens, in accordance with embodiments;

FIG. 42 shows a simulated image of a boy on the retina of the eye of FIG. 41, in accordance with embodiments;

FIG. 43 shows a spot diagram of different axial positions before and after the retina of the eye of FIG. 41, in accordance with embodiments;

FIG. 44 shows the RMS values for best focal position onto the retina of the eye in FIG. 41, in accordance with embodiments;

FIG. 45 shows a Ray Fan diagram for the eye of FIG. 41, in accordance with embodiments;

FIG. 46 shows a simulation of a model eye without lateral shift of the lens, in accordance with embodiments;

FIG. 47 shows the RMS values for best focal position onto the retina of the eye in FIG. 46, in accordance with embodiments;

FIG. 48 shows a simulation of a model eye with 0.5 mm lateral displacement of the lens, in accordance with embodiments;

FIG. 49 shows the RMS values for best focal position onto the retina of the eye in FIG. 48, in accordance with embodiments;

FIG. 50 shows a simulation of a model eye with 1.0 mm lateral displacement of the lens, in accordance with embodiments;

FIG. 51 shows the RMS values for best focal position onto the retina of the eye in FIG. 50, in accordance with embodiments;

FIG. 52 shows treatment zones for ELP adjustment, in accordance with embodiments;

FIG. 53 shows treatment zones for ELP adjustment, in accordance with embodiments;

FIG. 54A shows a treatment zone for phacotripsy, in accordance with embodiments;

FIG. 54B shows a method of ELP, in accordance with embodiments;

FIGS. 55-59 show the results of Experiment 1, in accordance with embodiments;

FIG. 55 shows the treatment zone for ELP adjustment used in Experiment 1, in accordance with embodiments;

FIG. 56 shows an image of the eye prior to ELP adjustment, in accordance with embodiments;

FIG. 57 shows an image of the eye after ELP adjustment, in accordance with embodiments;

FIG. 58 shows an OCT cross-sectional slice of the eye of FIG. 56, in accordance with embodiments;

FIG. 59 shows an OCT cross-sectional slice of the eye of FIG. 57, in accordance with embodiments;

FIGS. 60-64 show the results of Experiment 2, in accordance with embodiments;

FIG. 60 shows the treatment zone for ELP adjustment used in Experiment 2, in accordance with embodiments;

FIG. 61 shows an image of the eye prior to ELP adjustment, in accordance with embodiments;

FIG. 62 shows an image of the eye after ELP adjustment, in accordance with embodiments;

FIG. 63 shows an OCT cross-sectional slice of the eye of FIG. 61, in accordance with embodiments;

FIG. 64 shows an OCT cross-sectional slice of the eye of FIG. 62, in accordance with embodiments;

FIGS. 65-69 show the results of Experiment 3, in accordance with embodiments;

FIG. 65 shows the treatment zone for ELP adjustment used in Experiment 3, in accordance with embodiments;

FIG. 66 shows an image of the eye prior to ELP adjustment, in accordance with embodiments;

FIG. 67 shows an image of the eye after ELP adjustment, in accordance with embodiments;

FIG. 68 shows an OCT cross-sectional slice of the eye of FIG. 66, in accordance with embodiments;

FIG. 69 shows an OCT cross-sectional slice of the eye of FIG. 67, in accordance with embodiments;

FIGS. 70-74 show the results of Experiment 4, in accordance with embodiments;

FIG. 70 shows the treatment zone for ELP adjustment used in Experiment 4, in accordance with embodiments;

FIG. 71 shows an image of the eye prior to ELP adjustment, in accordance with embodiments;

FIG. 72 shows an image of the eye after ELP adjustment, in accordance with embodiments;

FIG. 73 shows an OCT cross-sectional slice of the eye of FIG. 71, in accordance with embodiments;

FIG. 74 shows an OCT cross-sectional slice of the eye of FIG. 72, in accordance with embodiments;

FIGS. 75-79 show the results of Experiment 5, in accordance with embodiments;

FIG. 75 shows the treatment zone for ELP adjustment used in Experiment 5, in accordance with embodiments;

FIG. 76 shows an image of the eye prior to ELP adjustment, in accordance with embodiments;

FIG. 77 shows an image of the eye after ELP adjustment, in accordance with embodiments;

FIG. 78 shows an OCT cross-sectional slice of the eye of FIG. 76, in accordance with embodiments; and

FIG. 79 shows an OCT cross-sectional slice of the eye of FIG. 77, in accordance with embodiments.

FIG. 80A shows OCT cross-sectional slices of an eye, in accordance with embodiments;

FIG. 80B shows OCT cross-sectional slices of an eye, in accordance with embodiments;

FIG. 81 shows temperature profiles of an eye treated with a laser beam with the eye coupled to a chilled lens, in accordance with embodiments;

### DETAILED DESCRIPTION OF THE INVENTION

A better understanding of the features and advantages of the present disclosure will be obtained by reference to the following detailed description that sets forth illustrative embodiments, in which the principles of embodiments of the present disclosure are utilized, and the accompanying drawings.

Although the detailed description contains many specifics, these should not be construed as limiting the scope of the disclosure but merely as illustrating different examples and aspects of the present disclosure. It should be appreciated that the scope of the disclosure includes other embodiments not discussed in detail above. Various other modifications, changes and variations which will be apparent to those skilled in the art may be made in the arrangement, operation and details of the method and apparatus of the present disclosure provided herein without departing from the spirit and scope of the invention as described herein.

The embodiments disclosed herein can be combined in one or more of many ways to provide improved methods and apparatus for treating the eye. The treated ocular tissue, or membranes or pathological transformations thereof, may comprise one or more of corneal tissue, lens tissue, scleral tissue, vitreal tissue, or zonulae extending between the lens capsule and the ora serrata.

As used herein like characters identify like elements.

As used herein A and/or B encompasses one or more of A or B, and combinations thereof such as A and B.

The methods and apparatus disclosed herein are well suited for adjusting the position of the lens of the eye in order to correct refractive error and other errors of the eye related to the refractive and wavefront properties of light. The methods and apparatus disclosed herein are well suited for combination with other treatments such as surgical implants, intraocular lenses, laser in situ keratomileusis (LASIK), photorefractive keratectomy, small incision lens extraction (SMILE), crosslinking and corneal crosslinking.

The embodiments as disclosed herein provide improved methods and apparatus for the treatment of one or more of presbyopia, glaucoma, effective lens position (ELP) adjustment (for example to treat myopia, hyperopia, or astigmatism), and combinations thereof. For example, presbyopia treatments as disclosed herein can have a beneficial effect on a patient's intraocular pressure (hereinafter “IOP”). Alternatively or in combination, the treatment can be directed to the treatment of glaucoma, for example. The treatments and apparatus disclosed herein can be combined with many known methods and apparatus for treatment. For example, the restoration of accommodation as described herein can be combined with one or more of many known prior accommodating intraocular lenses (IOLs), for example. Alternatively or in combination, the methods and apparatus as disclosed herein can be combined with one or more known glaucoma therapies. Although many embodiments are described with reference to a natural lens of the eye, the embodiments disclosed herein can be used to improve vision with IOLs.

Provisional Application to U.S. App. Ser. No. 61/801,041, filed on Mar. 15, 2013, incorporated herein by reference, discloses improved methods and apparatus to treat presbyopia and/or glaucoma in accordance with many embodiments disclosed herein. In many embodiments, tissue is not substantially removed and is moved to a new location with the treatment. This movement of collagenous tissue from a first location to a second location provides improved treatment with less regression of effect and healing. The methods and apparatus disclosed therein describe treatment of the eye without ablation and without formation of hard spots as can be formed when a laser removes tissue with heat. In many embodiments, the treatment can be performed without incisions of the eye, in order to decrease the invasiveness of the procedure and decrease regression of effect.

Examples of treatment modalities of the eye suitable for use with the systems and/or methods disclosed herein are described in PCT/US2014/023763, filed on 11 Mar. 2014, entitled “SCLERAL TRANSLOCATION ELASTO-MODULATION METHODS AND APPARATUS” (attorney docket no. 48848-703.601); U.S. provisional application 62/237,840, filed on 6 Oct. 2015, entitled “ULTRASOUND DIRECTED CAVITATIONAL METHODS AND SYSTEMS FOR OCULAR TREATMENTS” (attorney docket no. 48848-704.101); and U.S. provisional application 62/254,138, filed on 11 Nov. 2015, entitled “ULTRASOUND DIRECTED CAVITATIONAL METHODS AND SYSTEMS FOR OCULAR TREATMENTS” (attorney docket no. 48848-704.102); the entire disclosures of which are incorporated herein by reference.

In many embodiments, the methods and apparatus disclosed herein provide effective lens positioning adjustment (hereinafter “ELP adjustment”) of an eye in order to adjust the position of the lens for treatment of one or more of presbyopia, glaucoma, myopia, hyperopia, or astigmatism. As used herein, ELP adjustment refers of an adjustment of one or more of an effective position or an orientation of a lens of the eye.

Treatment Temperature/Operating Parameters

In many embodiments, the ELP adjustment procedure provides application of heat to the eye to produce a thermo-mechanical response in a tissue of the eye, such as in the cornea and/or sclera. For example, the cornea and/or sclera can be heated to a range from about 50 to about 70 degrees Centigrade, for example between 60 and 70 degrees Centigrade, to produce shrinkage of the tissue. Tissue may be heated within the range without substantially weakening the tissue. In many embodiments, a portion of the eye can be heated to a temperature within a range of up to about 55 or 60 degrees Centigrade in order to relax the tissue. Heating of the cornea and/or sclera to a temperature within this range can also produce softening and/or plasticizing (e.g., to approximately 10% of the native strength of the tissue). The cornea and/or sclera can be heated to greater than 80 degrees Centigrade of the eye to produce denaturation of the tissue. The tissue may be weakened by heating to a temperature within a range from about 70 to about 90 degrees Centigrade.

The heating of tissue can be controlled in order to provide desired amounts of shrinkage or relaxation and combinations thereof. For example, heating collagenous tissue such as scleral tissue to a temperature within a range from about 50 to 70 degrees C. can result in shrinkage of the tissue that can be effective to move the lens position. For temperatures in a range from about 60 to 70 degrees C., heating of the tissue can result in shrinkage or relaxation, depending on how long the tissue is heated. For example, heating tissue within a range from 60 to 70 degrees C. for shorter amounts of time can result in tissue shrinkage, while heating tissue for longer amounts of time can result in relaxation. To relax tissue, the tissue temperature can be elevated to a temperature in a range from about 60 degrees C. to about 80 degrees C. For example, heating tissue to about 80 degrees C. for about a millisecond can result in tissue relaxation. For lower temperatures within this 60 to 80 C range, the tissue can be heated for amounts of time longer than 1 ms to provide tissue relaxation.

The ultrasound methods and apparatus as disclosed herein can be used to heat tissue with similar temperatures and locations as described with reference to laser treatment, in order to provide ELP adjustment as described herein. Other types of energy as described herein can be used similarly to adjust the effective lens position as will be appreciated by one of ordinary skill in the art. The ultrasonic approaches as described herein can also be used to soften tissue without substantial heating, for example.

The cooling methods and apparatus disclosed herein can be combined with the energy sources described herein in order to decrease heating of tissue near external surfaces of the eye, such as conjunctival and epithelial layers of the eye.

In many embodiments, the methods and apparatus can be used in combination with ELP to treat one or more of many disorders of the eye, and can be used to treat many of these disorders with an energy source, under control of computer instructions. The apparatus can be used to one or more of soften or resect tissue with non-thermal treatments, for example less than about 50 degree Centigrade (degrees C.). Alternatively or in combination the methods and apparatus can be used in a thermal mode to treat tissue thermal with treatments more than about 50 degrees C., for example about 60 degrees C. or more. The non-thermal treatment can be used in many ways, such as for accurate tissue resection. The ultrasonic non-thermal tissue resection can be performed without substantial bubble formation, which allows the user such as a surgeon to accurately treat many regions of the, in many instances without interference from bubbles. In many instances, the mechanism of non-thermal treatment is substantially mechanical, such that tissue can be resected with very fine and accurate incision structures, which can be three dimensional. The ELP adjustment-induced cavitation can focally disrupt or liquefy or micro-porate (spongify) tissue and reduce rigidity, thus enhancing mobility of accommodative complexes, aqueous outflow facilities, and adjustment of ELP. By inducing cavitation non-thermally, the methods and system disclosed herein can provide improved safety over currently available thermal treatments.

Three or more types of treatments can be provided depending on energy delivery settings: 1. liquefaction, 2. paste or 3. vacuolated thermal treatment. Liquefied treatment regions are pure mechanically-disrupted treatment regions and can be observed for example with an ultrasound pulse duration less than 30 ms, which is slightly longer than time to boil. Paste treatments represent an intermediate state between mechanically-induced liquefaction and vacuolated thermal treatment. Paste treatment regions may be generated non-thermally, e.g. spongification, or thermally, as a pre-cursor state to vacuolated thermal treatment, or with a combination of both thermal and non-thermal settings. The use of chilled (4 degree C.) degassed Trehalose (optionally with NSAIDs) may be preferred over water as the coupling medium for improved ocular surface lubrication, in some embodiments.

The optional use of nanoparticles similar to nanoparticles for enhanced imaging can be used to enhance cavitation in some embodiments. Nanoparticles can be used with ultrasound treatment as disclosed herein to reduce the cavitational dosage requirements, for example by a factor of 2×-10×. The nanoparticles may comprise one or more of perfluorocarbon, lipid, albumin, or galactose, for example. Targeted (optionally drug-free) lysis due to microstreaming and micro-fragmentation (<5 um diameter) can improve micro-circulation and contains region of treatment demarcation with added safety. Treatments can be provided with decreased bleeding and decreased apoptosis, which can be shown with blood brain barrier and myocardial infraction studies, for example. While the nanoparticles can be used for any of the treatments disclosed herein such as glaucoma treatments, the nanoparticles can be beneficial for fractionation and apoptosis of choroidal neovascularization (CNV) and uveal melanomas, for example.

Drug delivery can be enhanced with the methods and apparatus disclosed herein. Debulking of tissue as disclosed herein can be used as a preparatory step and may be advantageously administered in combination drug delivery to promote drug delivery and improve delivery of the drug through the treated tissue.

The controlled cavitation as described herein can be provided with simultaneous imaging administered from the ELP apparatus to the tissue with a fluidic coupling path and a rotating arm to sequence the focused patterns within the tissue to deliver the drug at doses related to the scan rates of the ultrasound beam as disclosed herein.

The tissue can be treated so as to provide small zones from which the tissue is removed by natural processes such as macrophages in order to remove tissue. The removed tissue can be removed from several small locations so as to make the tissue more elastic, similar to a sponge.

The energy can be delivered so as to generate cavitation and increase elasticity of the target tissue with decreased amounts of heat. In many instances, the ELP adjustment treatment provides debulking of the tissue which increases the elasticity of the tissue. The amount of heating of the treated tissue can be controlled to be no more than about 10 degrees C., for example no more than about 5 degrees C., which can increase elasticity with decreased amounts of regression.

Treatment Locations

The present inventors have determined with finite element analysis (FEA) treatment regions suitable ELP adjustment. The treatment region can be located in scleral tissue and can be about 0.25 to about 0.75 mm deep, for example. The treatment can be located in one or more of the cornea and sclera, for example slightly below the epithelium and conjunctiva. The corneal treatment region can be from about 0.100 to about 400 um deep, for example.

When light energy is used, the depth of tissue with sufficient heating can depend on the wavelengths of light energy. For example, light energy having wavelengths in a range from about 1.9 to 2.1 um, the 1/e attenuation depth can be in a range from about 200 to 300 ums, for example about 225 to 275 um. For light energy having wavelength in a range from about 1.3 to 1.6 um, the 1/e attenuation depth is within a range from about 350 to 450 um. When combined with the cooling lens as described herein, the profile of tissue heating can result in a peak temperature that is located beneath the exterior surface of the ocular tissue, even though the amount of light energy absorbed near the surface is greater than the amount of light energy absorbed at the tissue location which undergoes the highest amount of temperature increase. The depth of tissue that shrinks or relaxes can have a profile extending to a depth in the tissue.

Presbyopia

The ELP treatment as described herein can be combined with presbyopia treatment of the eye. In many embodiments, the eye is treated such that the posterior vitreous zonules can move at least anteriorly to allow the lens capsule to move anteriorly or reshape, or both, in order to provide improved accommodation. In many embodiments, the eye is treated in order to provide improved anterior-centripetal movement of the ciliary body at the insertion of the posterior vitreous zonule into the ciliary body. Alternatively or in combination, the eye can be treated so as to increase the circumlental space between the ciliary body and lens capsule in order to provide increased amounts of accommodation. The increased amount of anterior movement of the posterior vitreous zonule from the unaccommodated state to the accommodative state can be within a range from about 250 to about 1000 um, for example.

The ELP adjustment methods and system can provide non-reductive and non-ablative restoration of accommodative power compatible with the Helmholtz theory of accommodation. Treatment can be applied to the eye from the scleral spur to the ora serrata while avoiding damage to limbal stem cells, conjunctiva, epithelium, and eye muscles. The ELP adjustment procedure can include elasto-modulation to one or more of: soften and/or plasticize scleral regions near the ciliary body apex to enhance inward movement of the ciliary body during accommodation; soften and/or plasticize scleral regions near the insertions of the posterior vitreous zonules to enhance anterior movement of the ciliary body during accommodation; or soften and/or plasticize regions of the sclera and/or cornea near the sclera spur to enhance corneal asphericity and/or flexing during accommodation.

The sclera can be softened posterior to the lens equator and anterior to the insertion location of the posterior zonules near the ora serrata in one or more of many ways in order to encourage movement of the posterior vitreous zonules at least anteriorly in order to provide improved accommodation, such as one or more of light energy, ultrasound energy, radiofrequency energy, electrical energy, thermal energy, electroporation, microwave energy, optoporation, photonic desincrustation, or galvanic desincrustation. In many embodiments, the softening of the scleral tissue posterior to the lens equator provides at least about one millimeter of anterior movement of the posterior vitreous zonules lens and/or capsule so as to provide at least about one diopter of accommodation. In many embodiments, the movement of the posterior vitreous zonules near the insertion into the ora serrata allows the lens to move anteriorly and to reshape itself with a more convex curvature. In many embodiments, the sclera is softened without removal of collagen from the tissue, which can inhibit regression of the softening effect. The softening of the sclera can be performed so as to inhibit damage to the ciliary body and choroid, and the energy such as light energy can be directed in a manner that avoids the ciliary body and choroid. The scleral softening can be performed such that the zonules of the eye comprise slack subsequent to treatment in order to inhibit changes in the position of the lens and/or capsule when the eye is configured for far vision and inhibit changes to the far vision refraction of the eye. In many embodiments, the posterior vitreous zonules comprise at least some slack in order to allow the lens capsule to move anteriorly. In many embodiments, the softened scleral tissue between the lens equator and insertion of the posterior vitreous zonules at the ora serrata moves interiorly toward an optical axis of the eye when the eye accommodates, and may provide inward movement of the posterior vitreous zonules. In many embodiments, the scleral tissue is translocated near the ciliary body apex in order to increase the circumlental space. The translocation of the scleral tissue and ciliary body apex can be performed without tissue removal, in order to decrease regression of an initial effect and in order to decrease the invasiveness of the procedure.

The embodiments disclosed herein provide improved accommodation of the eye with an increase of one or more of the perilenticular space or a softened and/or plasticized portion of scleral or corneal tissue. The perilenticular space extending between the ciliary body and the lens of the eye can be increased with tissue stabilization and shrinking. In some embodiments, the perilenticular space is increased with cross-linking of an outer portion of a sclera of the eye near a ciliary body of the eye so as to stabilize the outer portion of scleral tissue with increased stiffness, and an inner shrinking treatment of an inner portion of the sclera located inwardly from the outer portion and toward the lens of the eye. The shrinking of the inner portion can be combined with the stabilization of the outer portion such that the inner surface of the ciliary body is urged away from the lens capsule so as to increase the perilenticular space. The portion of softened and/or plasticized scleral tissue can be located between sclera disposed over the ora serrata and sclera corresponding to the equator of the lens of the eye in order to allow the lens capsule and lens to move an increased amount in order to restore accommodation. The softening and/or plasticizing of the scleral tissue portion can provide improved accommodation with increased mobility of the posterior vitreous zonules extending between the ciliary body and the ora serrata. In many embodiments, the stiffening of the outer portion of the sclera and shrinking of the inner portion of the sclera provides improved drainage of the channels of the trabecular meshwork of the eye, and can be related to increased channel sizes of this tissue structure.

Glaucoma

In many embodiments, tissue stabilization and shrinking can be used to treat glaucoma. An outer portion of the sclera can be treated with cross-linking to add stiffness and stabilize the outer portion. An inner portion disposed inwardly from the outer portion can be treated with shrinking in order to urge one or more tissue structures of the eye toward the stabilized portion and increase channel sizes of the one or more tissue structures of the eye such as Schlemm's canal and one or more channels of the trabecular meshwork.

In an aspect, a method is provided for treating an eye. The method can include cross-linking an outer portion of the eye and shrinking an inner portion of the eye, such that a tissue structure of the eye has moved outwardly toward the cross-linked outer portion when the inner portion has shrank. Outward can include radially outward away from an optical axis of the eye.

In many embodiments, the outer portion can include a sclera of the eye through which a plane defining an equator of the lens of the eye extends in order to treat a presbyopia of the eye. The outer sclera portion can include a cross-linked profile prior to shrinking. The cross-linked profile can be substantially maintained when the inner portion shrinks. A cross-sectional thickness of the sclera can extend from an outer surface of the sclera adjacent a conjunctiva to an inner surface of the sclera adjacent a trabecular meshwork through the outer portion and the inner portion. The cross-sectional thickness of the sclera can decrease from a first thickness prior to shrinking to a second thickness subsequent to shrinking, the second thickness less than the first thickness. The inner surface can include an inner surface profile extending along an inner side of the sclera. The outer surface can include an outer surface profile extending along an outer side of the sclera. The inner surface can deflect outwardly an amount greater than the outer surface deflects inwardly when the inner portion has shrunk

In many embodiments, the tissue structure of the eye can include a ciliary body of the eye in order to increase a perilenticular space of the eye. The tissue structure of the eye can include one or more of a portion of the cornea or a portion of the sclera lateral to the Schlemm's canal in order to increase a cross-sectional size of one or more of the Schlemm's canal or a trabecular meshwork of the eye in order to treat glaucoma of the eye. The tissue structure of the eye can include a portion of the sclera lateral to a trabecular meshwork of the eye in order to increase a cross-sectional size of channels of the trabecular meshwork in order to treat glaucoma of the eye.

In many embodiments, the eye includes a conjunctiva disposed over a sclera and the inner portion is treated through the conjunctiva of the eye. The eye can include a conjunctiva and the conjunctiva can be moved away from the sclera to treat the inner portion.

In many embodiments, the outer portion can be cross-linked with a cross-linking agent including one or more of: riboflavin, rose bengal, methylene blue, indocyanine green, genipin, threose, methylglyoxal, glyceraldehydes, aliphatic β-nitro alcohols, or black currant extract, or an analog of any of the above. The cross-linking agent can include one or more of a chemical agent or photosensitizers. The inner portion can shrink with one or more of thermal energy, radiofrequency energy, electrical energy, microwave energy, light energy, or ultrasound energy. The energy source can include a microelectrode array. The cross-linker can include a chemical photosensitizer. The method can include placing a heat sink over the outer portion to conduct heat away from the outer portion when the inner portion is heated. The inner portion can shrink with application of energy and the heat sink can include a material transmissive to wavelengths of the light energy in order to heat the tissue with light energy absorbed beneath the heat sink. The inner portion can be heated to a temperature within a range from about 50 to about 70 degrees Centigrade in order to shrink the tissue. The portion can be heated within the range without substantially weakening the tissue. A layer of conjunctiva located above the inner portion can remain substantially viable when the inner portion is treated in order to inhibit pain and swelling.

In many embodiments, the energy source can include a light energy source, in which the light energy source configured to emit at least one wavelength of light to cross-link the outer portion and shrink the inner portion. The light source can include a single light source to emit a wavelength of light to cross-link the outer portion and shrink the inner portion, optionally, to shrink the inner portion and cross-link the outer portion together, or optionally, to shrink the inner portion after the outer portion has been cross-linked, and combinations thereof. The light source can include a first light source to cross-link the outer portion and a second light source to shrink the inner portion. The first light source can be configured to emit a first light energy including a first wavelength of light and the second light source can be configured to emit a second light energy including a second wavelength of light, the first wavelength different from the second wavelength. The light source can include a softening light source to soften a tissue of the sclera.

In many embodiments, the method can include softening a portion of scleral tissue of the eye, the sclera tissue extending posterior to an equatorial plane of a lens of the eye and anterior to an insertion location of posterior vitreous zonules proximate an ora serrata of the eye. The portion can be heated to a temperature within a range from about 70 to about 90 degrees Centigrade in order to weaken the tissue. The softened portion can include four softened portions, each corresponding to four locations away from muscles of the eye including inferior muscles, superior muscles, nasal muscles, and temporal muscles in order to inhibit damage to the muscles.

ELP

In many embodiments, the ELP adjustment system and methods may be used to adjust one or more of an effective position or an orientation of a lens of an eye. An energy source may be configured to deliver energy to the eye in order to one or more of shrink or relax the sclera of the eye. The energy source may be configured to move the lens anteriorly to treat farsightedness. The energy source may be configured to move the lens posteriorly to treat nearsightedness. The energy source may be configured to tilt the lens to treat astigmatism. In many embodiments, the energy source may be configured to treat one or more of nearsightedness, farsightedness, or astigmatism by moving the lens one or more of anteriorly, posteriorly, or by tilting.

The ELP adjustment system and methods may be used to delivery energy to the sclera of the eye to move the lens with respect to a target lens position or target lens orientation. The target lens position or target lens orientation may correspond to a target plane of the lens.

The ELP adjustment system and methods may be used to induce equatorial scleral translocation above and below the target plane in order to adjust the ELP of an IOL after insertion, for example. Post-operative ELP errors may for example occur due to IOL shifts, pre-operative calibration calculation errors, capsular shrinkage, extra-lenticular realignment, or any combination thereof. When HIFU is used, the ELP adjustment system may be used in mechanical mode to generate balanced fractionation or shrinkage along the sclera for 360 degrees about the target plane resulting in stretching or yielding of the sclera and ELP adjustment, for example. Alternatively or in combination the ELP adjustment system may be operated in thermal mode to induce annular shrinkage of the sclera.

In many embodiments, ELP adjustment may for example include translation, tilting, centering, or any combination thereof due to regional scleral shrinkage (elastification) or relaxation (compliance) about a line along a target plane. The ELP adjustment system and methods may be used to deliver energy to generate one or more shrinkage regions, one or more relaxation regions, or any combination thereof, with respect to the target plane of the lens. In many embodiments, the shrinkage region and the relaxation region are arranged on opposite sides of the target plane so as to urge the lens toward the target position. In some embodiments, the relaxation region of the sclera corresponds to a location of an equator of the lens prior to treatment such that the lens moves away from the relaxation region toward the stiffened region with treatment. In many embodiments, the shrinkage region and the relaxation region are arranged on the same side of the target plane and on opposite sides of the eye in order to urge the lens toward the target location or orientation with treatment. The lens may rotate away from the relaxation region on a first side of the eye and rotate toward the shrinkage region on the second side of the eye in order to adjust the tilt of the lens with treatment.

Treatment may be focused to a region extending from about 1 mm anterior to the anterior equatorial zonulae scleral insertion to about 1 mm posterior to the posterior equatorial zonulae scleral insertion. The anterior zonulae scleral insertion generally corresponds to the location of the insertion of anterior zonulae in to the ciliary body, and the location of the sclera immediately above this location. The posterior zonulae scleral insertion generally corresponds to the location of the insertion of posterior zonulae in to the ciliary body, and the location of the sclera immediately above this location. Alternatively or in combination, one or more treatment zones may include one or more of the sclera, the trabecular meshwork, and around Schlemm's Canal. Scleral shrinkage may be induced on the side of the target plane further from the lens equator while scleral relaxation may be induced on the side of the target plane closest to the lens equator so as to urge the lens equator towards the target plane. ELP adjustment using the ELP adjustment system described herein may occur without changing axial length or corneal curvature.

In many embodiments, ELP adjustment is used to treat myopia or hyperopia by facilitating movement of the lens away from or towards the cornea, respectively. The treatment region may comprise an annular region extending 360 degrees about the eye. The treatment region be arcuate and extend less than 360 degrees about the eye, for example 180 degrees. Alternatively or in combination, ELP adjustment may be used to treat astigmatism or coma by facilitating movement of the lens at an angle relative to the target plane. The effective lens angle (ELA) may be adjusted for example by inducing one or more of arcuate shrinkage and relaxation for 180 degrees about the eye and 180 degree mirrored arcuate relaxation and shrinkage. In some embodiments ELA may be adjusted by treating in a distinct symmetric orthogonal bowtie pair pattern with meridional degree-based shrinkage and relaxation. ELP adjustment treatment may be symmetrical or asymmetrical about the eye as needed to induce the desired changes in ELP. Treatment may be regional, for example to induce regional tip or tilt of the lens.

The ELP adjustment system and methods may be used to deliver energy to a scleral shrinkage region posterior to the lens equator in order to urge the lens posteriorly to treat myopia. The ELP adjustment system and methods may be used to deliver energy to a scleral relaxation region anterior to the lens equator in order to urge the lens posteriorly to treat myopia. The ELP adjustment system and methods may be used to deliver energy to a scleral shrinkage region anterior to the lens equator in order to urge the lens anteriorly to treat hyperopia. The ELP adjustment system and methods may be used to deliver energy to a scleral relaxation region posterior to the lens equator in order to urge the lens anteriorly to treat hyperopia. The ELP adjustment system and methods may be used to deliver energy to a scleral shrinkage region anterior to the lens equator on a side of the eye in order to tilt the lens with anterior movement of a portion of the lens on the side of the eye. The ELP adjustment system and methods may be used to deliver energy to a scleral relaxation region posterior to the lens equator on a side of the eye in order to tilt the lens with anterior movement of a portion of the lens on the side of the eye. The ELP adjustment system and methods may be used to deliver energy to a scleral shrinkage region posterior to the lens equator on a side of the eye in order to tilt the lens with posterior movement of a portion of the lens on the side of the eye. The ELP adjustment system and methods may be used to deliver energy to a scleral relaxation region anterior to the lens equator on a side of the eye in order to tilt the lens with posterior movement of a portion of the lens on the side of the eye.

The ELP adjustment system and methods may be used to deliver an annular pattern of shrinkage energy anterior to the lens equator so as to move the lens anteriorly. The ELP adjustment system and methods may be used to deliver an annular pattern of shrinkage energy posterior to the lens equator so as to move the lens posteriorly. The ELP adjustment system and methods may be used to deliver an annular pattern of relaxation energy anterior to the lens equator so as to move the lens posteriorly. The ELP adjustment system and methods may be used to deliver an annular pattern of relaxation energy posterior to the lens equator so as to move the lens anteriorly.

Light Energy Sources

The ELP adjustment system may comprise an energy delivery system configured to delivery energy to the eye. The energy delivery system may be configured to deliver the energy to one or more of a relaxation region of the sclera or a shrinkage region of the sclera, in order to move the lens anteriorly to treat farsightedness, posteriorly to treat nearsightedness, or to tilt the lens to treat astigmatism, and optionally combinations thereof. One or more of the energy source, processor, or energy delivery system may be configured to deliver energy to the eye.

In many embodiments, the ELP adjustment procedure provides extra-corneal and/or extra-lenticular energy treatment to soften and/or plasticize the sclera and/or peripheral cornea, such as with one or more of light energy, ultrasound energy, high intensity ultrasound energy, mechanical energy, radiofrequency energy, electrical energy, thermal energy, electroporation, microwave energy, optoporation, photonic desincrustation, or galvanic desincrustation.

The energy can be delivered with an optical or ultrasound delivery system, and the system may comprise an energy delivery system to direct energy to the eye or a hand held probe, and combinations thereof, for example.

In many embodiments, light energy is used to soften the tissue, and the light energy comprises wavelengths that are strongly absorbed by the collagen of the sclera or the water of the sclera, or both for example. In many embodiments, the light energy comprises wavelengths that are absorbed more strongly by stromal tissue than water, for example light comprising a wavelength within a range from about 4 to 6 um, such as from about 5.5 to 6.6 um. The light energy absorbed more strongly by stroma than water has the advantage of providing more accurate treatment with less interference with water, and can allow the tissues of the eye to retain healthy amounts of water during treatment, for example tissues of the conjunctiva of the eye. Also, interference from water based surgical fluids such as saline and anesthetics can be substantially inhibited.

In many embodiments, the light energy comprises wavelengths within a range from about 1 to 6 um, such as from about 1 to 3 um. In many embodiments the light energy comprises wavelengths within a range from about 1.4 to about 2 um, for example about 1.46 um or 2.01 um, and other ranges as described herein.

In many embodiments a heat sink is provided to couple to the conjunctiva and the heat sink comprises a material transmissive to the light energy, such as sapphire or Zinc Selenide (hereinafter “ZnSe”). The heat sink material can be configured to transmit light energy absorbed more strongly by the stroma than water, and may comprise Zinc Selenide (hereinafter “ZnSe”), for example. The heat sink can be chilled to inhibit damage to the conjunctiva of the eye. The heat sink can provide improved transmission of light energy when condensation is present, as the condensed water may be less strongly absorbed by the laser beam. In many embodiments, one or more layers of the epithelium of the eye (basal layer, wing layer or squamous layer) remains substantially intact above the zone where the eye has been treated, for example at least one layer of viable epithelial cells can remain intact when the heat sink is removed.

In many embodiments, the optically transmissive material of the heat sink is shaped and optically configured with smooth surfaces so as to comprise an optically transparent heat sink such as a lens. The heat sink may comprise a window of the optically transmissive material, and can be one or more of many shapes such as a flat on opposing surfaces, plano-concave, or convex-concave. The convex-concave heat sink window may comprise a meniscus shaped lens, having substantial optical power or no substantial optical power, for example.

The location of the heat sink can be fixed in relation to a fixed structure of the laser system in order to fix the location of the eye, and the heat sink may comprise one or more curved surfaces such as a concave surface to engage the eye. In many embodiments, an arm extends from the fixed structure of the laser system to the heat sink in order to fix the location of the heat sink.

The laser may comprise one or more of many lasers emitting one or more of many wavelengths, such as infrared lasers. In many embodiments, the laser comprises a quantum cascade laser configured to emit light having a wavelength within a range from about 5.8 to about 6.6 um, for example from about 6 to about 6.25 um. In many embodiments, the laser comprises a quantum cascade laser or continuous wave laser configured to emit light having a wavelength within a range from about 1 to about 6 um, such as from about 1 to 3 um. In many embodiments the laser is configured to emit light having a wavelength within a range from about 1.4 to about 2 um, for example about 1.46 um or 2.01 um, and other wavelength ranges as described herein. Such lasers are commercially available, and can be configured by a person of ordinary skill in the art for treatment of the eye as described herein.

In many embodiments the ELP adjustment treatment apparatus comprises an energy source such as a laser and a docking station to retain the eye. In many embodiments the docking station comprises a chilled optically transmissive heat sink to couple to the eye. The docking station couples to the eye such that the heat sink contacts the conjunctiva of the eye and fixes the working distance of the eye relative to the surgical laser, such that the scleral treatment comprising softening posterior to the lens equator can be performed accurately. In many embodiments, heat sink is chilled such that at least one epithelial layer of the conjunctiva of the eye above the treated tissue remains viable, in order to expedite healing of the eye and decrease invasiveness of the procedure. The chilled heat sink structure can be chilled to a temperature within a range from above the freezing temperature of the eye and saline, at about −3 degrees Celsius (C), to below an ambient room temperature of about 20 degrees Celsius. Alternatively, a heat sink can be provided without chilling. In many embodiments, the freezing temperature of the eye corresponds to the freezing temperature of saline, about −3 degrees, for example. In many embodiments, the apparatus comprises a scanner to scan the laser beam. The laser beam can be pulsed or continuous, and in many embodiments comprises a continuous laser beam configured to inhibit temperature spikes related to ablation of the eye. In many embodiments the laser irradiance comprises a temporal and spatial profile to inhibit transient heating peaks of the tissue that can be related to removal of tissue such as ablation. The scanner can be configured to scan the laser beam in a plurality of quadrants, such as for quadrants with untreated regions between each of the quadrants to inhibit damage to muscles of the eye located between the treatment quadrants.

Other Energy Sources

While reference is made to softening tissue with light energy, other forms of energy can be used to soften tissue such as one or more of ultrasound energy, electroporation, microwave, thermal, electrical energy or di-electrophoretic energy and combinations thereof. In many embodiments, electroporation needles can be provided with a shaped array having four quadrants sized to extend through the conjunctiva and deliver electroporation energy beneath the conjunctiva. Alternatively, shaped contact electrodes can be provided without needles such that the current is passed through the epithelial layer of the conjunctiva to targeted regions of the sclera in order to soften at least a portion of the scleral tissue between the lens equator and insertion location of the posterior vitreous zonules. The electroporation to soften the sclera comprises an oscillating electric field to pass current in an electroporation treatment profile similar to the optical treatment profile disclosed herein.

Ultrasound Energy Sources

The energy source may be an ultrasound energy source, for example an ultrasound phased array. The ultrasound phased array can be programmed to treat non-adjacent focal zones with a very high duty cycle, e.g. greater than about 50% from the phased array, while each of focal treatment zones has a duty cycle less than about 5%, for example 2.5% or less in order to provide non-thermal tissue resection with very high pulse repetition frequencies in order to decrease treatment time. The phased array can also be used for imaging the tissue during treatment with imaging ultrasound from the array.

Due to the benefits of sub-surface tissue debulking/softening, high frequency histotripsy transducers, such as preferably electronically steerable phased array 5 MHz-20 MHz HIFU transducers can be used at under 250 W and with pulses within a range from 100 nsecs to 100 msec pulses. The pulse frequency can be under 1000 Hz repetition rates for sequential and non-sequential ocular treatments as described herein.

Using a customized deposition nomogram, temperature outside of the histotripsy focal zone may not exceed 50 degrees C. thus protecting tissue at depth. Negative acoustic pressures of up to −80 MPa (typically −30 MPa) can be provided and can be sufficient to provide a 10% debulking rate for a 360 degree treatment 3 minutes long.

The methods and system disclosed herein can provide an energy source such as a high intensity focused ultrasound (HIFU) energy source for treatment of tissue so as to increase elasticity of the tissue. The methods and system disclosed herein may utilize HIFU treatment to induce cavitation in a non-incisional and non-thermal manner. HIFU tissue penetration is not dependent on the opacity of the tissue, therefore HIFU may have greater access to tissue than laser systems which cannot penetrate through opaque media. Additionally, by inducing cavitation non-thermally with HIFU, the methods and system disclosed herein may prevent boiling bubble formation during cavitation and subsequent opacification of treated tissue.

The increased elasticity of the tissue can be provided at locations arranged in order to provide a therapeutic effect, such as ELP adjustment, with decreased amounts of regression. In many embodiments, the ultrasound beam can be focused to a small spot size with a frequency within a range from about 5 to 25 MHz (mega Hertz) in order to provide improved accuracy as shallow locations such as 1 mm or less below a surface of the eye, for example within a range from about 0.1 to about 0.9 mm.

The methods and system disclosed herein can provide a focused spot having a cross-sectional size within a range from about 50 um to about 200 um full width half maximum (FWHM); the corresponding cavitation can be similarly sized within similar ranges. The energy beam, for example an ultrasound beam, can be focused and pulsed at each of a plurality of locations to provide a plurality of cavitation zones at each of the target regions. Each pulse may comprise a peak power within a range generating focal negative peak pressures of about 30 MPa (mega Pascals). While the treatment pulses can be arranged in many ways within a region, in many instances the pulses can be spaced apart within a region to provide intact tissue such as intact sclera between pulses. Alternatively or in combination, the pulses can be overlapped to provide an overlapping treatment region having dimensions within a range from about 100 um to about 1 mm, and a plurality of spaced apart treatment regions can be provided within a treatment location. The depth of the treatment can be controlled in accordance with the region being treated. For example, glaucoma treatments of Schlemm's canal can be about 0.5 mm or less, and treatment regions of the ora serrata which can be deeper, for example within a range from about 0.5 to about 1.0 mm deep. For treatments located along the ciliary apex the treatment can be within a range from about 0.25 mm to about 0.75 mm.

The treatment geometry can be arranged in many ways and may comprise a length within a range from 100 um to 1 mm, a volumetric regions within a range from (400 um×100 um×360 degrees) and durations of exposure of less than 3 minutes are easily managed with a motorized circumferential track and 5 MHz-10 MHz theranostic applicator.

Use of dual frequency ultrasound with a low frequency pump combined with high frequency ultrasound can be used to reduce the high frequency cavitation threshold.

The methods and apparatus can be configured in many ways to treat tissue. This system can be configured to generate one or more of liquefaction, or vacuolated tissue, for example. The ELP adjustment system can be configured to provide mechanical erosion of collagen with breaking of the collagen fibers with well-defined margins using an energy source, for example one or more of light energy, ultrasound energy, radiofrequency energy, electrical energy, thermal energy, electroporation, microwave energy, optoporation, photonic desincrustation, or galvanic desincrustation.

The following boiling histotripsy parameters and responses describe an upper treatment limit using ultrasound energy in accordance with examples. The treatment energy can be substantially lower than shown in Table 1 below. Alternatively, treatment parameters similar to those shown in Table 1 can be used with decreased amounts of time.

TABLE 1
Boiling Histotripsy parameters and histopathology responses

| | | | | | | | Border | | |
|---|---|---|---|---|---|---|---|---|---|
| | Pulse | | Duty | | | Protein | Destroyed | | Effect Within |
| | Duration | | Factor | PRF | | Denaturation | vs Intact | | Lesion |
| Liquefied | <30 | ms | <.02 | 1 | Hz | 0% | <40 | um | mechanical |
| Paste | <100 | ms | <0.2 | <2 | Hz | 22%-27% | <40 | um | mechanical & |
| | | | | | | | | | thermal |
| Vacuolated | >100 | ms | >0.2 | >2 | Hz | 70%-90% | <100 | um | thermal |

The ELP adjustment system may be operated in mechanical mode to produce purely mechanical effects or in thermal mode to produce thermal effects in the tissue when exposed to an energy source such as HIFU. Mechanical mode using HIFU comprises a duty cycle of less than 2.5%, more preferably less than 1%. Thermal mode comprises a duty cycle of more than 2.5%. The device may be operated in either mechanical mode or thermal mode and may be readily switched between the two modes.

HIFU may be operated with a duty cycle range of about 0.1% to about 1% for cavitational histotripsy or a duty cycle range of about 1% to about 2.5% for boiling histotripsy depending on the energy applied by the HIFU system described herein. HIFU may be operated with a duty cycle range of about 0.01% to about 1% for cavitational histotripsy or a duty cycle range of about 1% to about 2.5% for boiling histotripsy depending on the energy applied by the HIFU system described herein.

The methods and system described herein may be operated with any combination of the parameters listed in Table 2.

TABLE 2
Treatment parameters.

| Parameter | Range | Preferred | |
|---|---|---|---|
| HIFU frequency | 750 kHz to 25 MHz | 10 | MHz |
| Total treatment | 0 min to 10 min | 4 | min |
| duration | | | |
| PRF | 1 Hz to 1000 Hz | 1000 | Hz |
| Non-thermal duty | 0.1% to 2.5% | 1% | |
| cycle | | | |
| Negative acoustic | −10 MPa to −80 MPa | −30 | MPa |
| pressure | | | |
| Tissue temperature | 37° C. to 100° C. | 41° | C. |
| Treatment size | 100 um × 400 um | May be configured to scan | |
| | (per focal point) | and treat multiple regions | |
| | | with focal points | |
| Treatment depth | 0 cm to 2.5 cm | 1 | cm |

| | | |
|---|---|---|
| Focal gain | 10 to 100 | Typical: 50 |

The ELP adjustment system may be operated at a HIFU frequency within a range of about 750 kHz to about 25 MHz, for example within a range of about 1 MHz to about 25 MHz, preferably within a range of about 5 MHz to 15 MHz, more preferably within a range of about 5 MHz to 10 MHz, more preferably about 10 MHz. The HIFU frequency for example may be within a range of about 2 MHz to about 24 MHz, for example within a range of about 3 MHz to about 23 MHz or within a range of about 4 MHz to about 22 MHz. The frequency for example may be within a range of about 5 MHz to about 21 MHz, within a range of about 6 MHz to about 20 MHz, or within a range of about 7 MHz to about 19 MHz. The frequency may for example be within a range of about 8 MHz to about 18 MHz, within a range of about 9 MHz to about 17 MHz, or within a range of about 10 MHz to about 16 MHz. The frequency may for example be within a range of about 11 MHz to about 15 MHz, within a range of about 12 MHz to about 14 MHz, or within a range of about 10 MHz to about 13 MHz.

The total ELP adjustment treatment duration may be up to 10 minutes, for example within a range from about 1 min to about 10 min, preferably about 4 min. The total treatment duration may for example be within a range of about 2 min to about 9 min, within a range of about 3 min to about 8 min, or within a range of about 4 min to about 7 min. The total treatment duration may for example be within a range of about 5 min to about 6 min. The total treatment duration may for example be within a range of about 2 min to about 6 min, preferably within a range of about 3 min to about 5 min, or within a range of about 4 min to about 6 min, and more preferably within a range of about 4 min to about 5 min. The total treatment duration for example may be within a range of about 3 min to about 10 min, or within a range of about 4 min to about 8 min.

The PRF of the HIFU energy source of the ELP adjustment system described herein may be within a range of about 1 Hz to about 1000 Hz, for example within a range of about 50 Hz to about 1000 Hz, preferably about 1000 Hz. The PRF may for example be within a range of about 100 Hz to about 900 Hz, within a range of about 200 Hz to about 800 Hz, or within a range of about 300 Hz to about 700 Hz. The PRF for example may be within a range of about 400 Hz to about 600 Hz, for example about 500 Hz to about 600 Hz. The PRF for example may be within a range of about 100 Hz to about 1000 Hz, preferably within a range of about 200 Hz to about 1000 Hz, more preferably within a range of about 500 Hz to about 1000 Hz.

The non-thermal duty cycle of the HIFU energy source of the ELP adjustment system described herein may be within a range of about 0.1% to about 2.5%, preferably less than 1.0%. The non-thermal duty cycle may for example be within a range of about 0.2% to about 2.4%, within a range of about 0.3% to about 2.3%, or within a range of about 0.4% to about 2.2%. The non-thermal duty cycle may for example be within a range of about 0.5% to about 2.1%, within a range of about 0.6% to about 2.0%, or within a range of about 0.7% to about 1.9%. The non-thermal duty cycle may for example be within a range of about 0.8% to about 1.8%, within a range of about 0.9% to about 1.7%, or within a range of about 1.0% to about 1.6%. The non-thermal duty cycle may for example be within a range of about 1.1% to about 1.5%, within a range of about 1.2% to about 1.4%, or within a range of about 1.2% to about 1.3%. The non-thermal duty cycle may for example be within a range of about 0.5% to about 1.5%, preferably within a range of about 0.7% to about 1.3%, more preferably within a range of about 0.8% to about 1.2%.

The number of cycles of the ELP adjustment system described herein may be within a range of about 1 to about 100 cycles, for example about 10 to about 100 cycles. The number of cycles may be within a range of about 20 to about 100 cycles, for example about 30 to about 100 cycles, for example about 40 to about 100 cycles. The number of cycles may be within a range of about 50 to about 100 cycles, for example about 60 to about 100 cycles, for example about 70 to about 100 cycles. The number of cycles may be within a range of about 80 to about 100 cycles, for example about 90 to about 100 cycles. The number of cycles may be within a range of about 10 to about 50 cycles, for example about 10 to about 30 cycles. The number of cycles may be within a range of about 10 to about 80 cycles, for example about 20 to about 50 cycles.

The peak negative acoustic pressure of the HIFU energy source of the ELP adjustment system described herein may be within a range of about −10 MPa to about −80 MPa, preferably about −30 MPa. The negative acoustic pressure may for example be within a range of about −20 MPa to about −70 MPa, within a range of about −30 MPa to about −60 MPa, or within a range of about −40 MPa to about −50 MPa. The negative acoustic pressure may for example be within a range of about −10 MPa to about −50 MPa, preferably within a range of about −20 MPa to about −40 MPa, more preferably about −30 MPa.

The negative acoustic pressure of the HIFU energy generated at the cornea by the ELP adjustment system may for example be calculated using the formula:

$${P}_{C} = {P}_{F} \frac{{A}_{F}}{{A}_{C}} \qquad ( 1 )$$

Where Pc=pressure at the cornea, P_{F}=pressure at the focal point of the HIFU energy, A_{F}=area of the focal point, and A_{C}=area of the cornea in line of the HIFU energy beam. The diameter of the focal point may for example be in a range from about 50 μm to 200 μm, thus the area of the focal point may be calculated to be about 1964 μm² to about 31416 μm². The negative pressure at the focal point may for example be in a range from about −10 MPa to about −80 MPa. The diameter of the cornea in the line of the HIFU beam may for example be about 3 mm, thus the area of the cornea may be about 7.07 mm². Using Formula 1 to calculate the pressure at the cornea given the exemplary ranges described, the negative acoustic pressure at the cornea may for example be within a range of about 2.8 kPa to about 356 kPa.

The negative acoustic pressure at the cornea may for example be within a range of about 1 kPa to about 350 kPa, for example within a range of about 1 kPa to about 300 kPa. The negative acoustic pressure at the cornea may for example be within a range of about 1 kPa to about 250 kPa, for example about 1 kPa to about 200 kPa. The negative acoustic pressure at the cornea may for example be within a range of about 0 kPa to about 150 kPa, for example about 1 kPa to about 100 kPa. The negative acoustic pressure at the cornea may for example be within a range of about 1 kPa to about 50 kPa, for example about 1 kPa to about 10 kPa.

The temperature of the tissue treated with the ELP adjustment system may be within a range of about 37° C. to about 100° C., preferably 41° C. The temperature of the tissue may for example be within a range of about 37° C. to about 50° C., preferably within a range of about 37° C. to about 45° C., more preferably within a range of about 37° C. to about 44° C., still more preferably within a range of about 37° C. to about 41° C.

The treatment size per focal point may be about 100 um×400 um. The ELP adjustment system described herein may be configured to scan and treat multiple regions with multiple focal points, thus the total treatment area may be any area of any size within the eye.

The treatment depth of the ELP adjustment system described herein may be within a range of about 0 cm at the surface of the eye to about 2.5 cm deep within the eye, preferably about 1 cm depending on the target tissue. The treatment depth may for example be within a range of about 0.1 cm to about 2.4 cm, within a range of about 0.2 cm to about 2.3 cm, or within a range of about 0.3 cm to about 2.2 cm. The treatment depth may for example be within a range of about 0.4 cm to about 2.1 cm, within a range of about 0.5 cm to about 2.0 cm, or within a range of about 0.6 cm to about 1.9 cm. The treatment depth may for example be within a range of about 0.7 cm to about 1.8 cm, within a range of about 0.8 cm to about 1.7 cm, or within a range of about 0.9 cm to about 1.6 cm. The treatment depth may for example be within a range of about 1.0 cm to about 1.5 cm, within a range of about 1.1 cm to about 1.4 cm, or within a range of about 1.2 cm to about 1.3 cm. The treatment depth may for example be within a range of about 0.25 cm to 0.75 cm, within a range of about 0.5 cm to about 1.5 cm, or 0.5 cm or less. The treatment depth is determined by the location of the tissue being treated.

The focal gain of the ELP adjustment system described herein may be within a range of about 10 to 100, for example within a range of about 20 to 90. The focal gain may for example be within a range of about 30 to 80, within a range of about 40 to 70, or within a range of about 50 to 60.

The voltage of the HIFU energy source of the ELP adjustment system described herein may be within a range of about 100V to about 400V, for example about 150V to about 350V. The voltage of the HIFU energy source of the ELP adjustment system described herein may be within a range of about 200V to about 300V, for example about 200V to about 250V.

Imaging while Treating

The methods and system disclosed herein can be used in many ways and can be used to image the tissue during treatment. The ELP adjustment treatment system may comprise in imaging apparatus such that the treatment can be combined imaging with one or more of magnetic resonance (MR) imaging, ultrasound biomicroscopy (UBM), ultrasound (US) imaging, optical coherence tomography (OCT), optical coherence elastography (OCE), or US elastography transducer measurements. The imaging apparatus can be combined with the ELP adjustment treatment with either simultaneous oblique trans-iridional imaging or on the coaxial therapeutic probe; and diagnostic images that are useful intra-operatively, for visualization as well as for feature/landmark tracking. Rapid real time MR images can be acquired when time-synchronized to ELP adjustment energy pulses with weighting motion gradients turned ON for greater cavitational sensitivity. MR/OCT/US guided ELP adjustment guidance can include one or more of pretreatment planning, image-based alignment and siting of the ELP energy source focus, real-time monitoring of treatment energy-tissue interactions, or real-time control of exposure and damage assessment.

The ELP adjustment treatment system may comprise an imaging apparatus capable of determining tissue elasticity before, during, or after ELP adjustment treatment, or some combination thereof, for example OCE or US elastography transducers. The treatment system may additionally or in combination comprise a mechanism for real-time temperature sensing, for example using an OCT transducer, in order for real-time monitoring of HIFU-induced temperature changes or to provide for control of HIFU exposure to maintain temperature.

Motorized diagnostic imaging in sync with histotripsy patterning can be achieved in these configurations. For example, real-time imaging of treatment tissue may allow for user input to a grid of target regions, which may be larger than the area covered by a single treatment or include multiple areas not in direct contact with each other, for motorized control of multiple treatments over a larger area, allowing the user to avoid manual repositioning which may save time and prevent mistakes.

Imaging may be configured to occur simultaneously with treatment. A processor can be coupled to the ultrasound array and configured with instructions to scan the beam to a plurality of locations and image the tissue during treatment. The system may also comprise a display coupled to the processor that allows the user to see the tissue treated on the display and to plan the treatment. The images shown on the display can be provided in real time and can allow the operator to accurately align the tissue with the treatment and may allow the operator to visualize the treatment area, and other locations away from the treatment area. The imaging of the treatment area can be used to determine identify the target area on the screen and to program the treatment depth and location in response to the images shown on the display. The imaging can be used to visualize movement of ocular structures during treatment in order to detect beneficial treatment effects. The processor can be configured with instructions to treat the eye with a first wavelength of ultrasound and to image the eye with a second wavelength longer than the first wavelength. The processor may alternatively or in combination be configured with instructions to treat the eye with HIFU and to image the eye with an embedded imaging apparatus, for example an optical coherence tomography (OCT) probe. The processor coupled to the array can be configured with instructions to provide both ultrasound wavelengths from the array. The imaging apparatus may provide additional tissue feedback data in real-time, for example temperature or elasticity.

The processor may be configured with instructions to determine the effective position or orientation (e.g. ELP) of the lens of the eye. The processor may determine a direction of movement or rotation of the lens toward the ELP. In response to the direction of movement or rotation of the lens, the processor may deliver one or more of shrinkage energy to a region of the sclera to shrink the sclera or relaxation energy to a region of the sclera to relax the sclera. Shrinkage energy may for example be delivered to a first region of the sclera to shrink the sclera and relaxation energy may be delivered to a second region of the sclera to relax the sclera in response to the determined direction of movement or rotation of the lens toward the ELP.

Advantages of ELP Adjustment Procedure

The ELP adjustment procedure may provide one or more of the following advantages:

Increased depth of field of the eye;

Preservation of distance visual acuity, as the central corneal and central lenticular regions are substantially unaffected by the treatment;

Preservation of limbal stem cells, ciliary muscle function, conjunctiva, epithelium, and aqueous production, as these are substantially unaffected by treatment;

No substantial loss of contrast sensitivity;

No substantial disturbances of night vision;

Preservation of aesthetics of the eye, as there are no cuts, implants, or full punctures of the eye;

Rapid patient recovery, as the conjunctiva is protected during treatment;

Tolerable treatment procedure for many patients;

Improved safety of the treatment procedure;

Avoidance of dry eye;

Sparing of cornea tissue from damage;

Non-subtractive treatment of corneas allowing for treatment of thin corneas;

Smoothing of corneal striae (e.g. SRI);

Smoothing of capsular striae (e.g. cSRI);

Circularization of pupil;

Centering of lenticular sub-luxation;

Correction of lenticular astigmatism;

Correction of mild to moderate refractive errors (e.g. myopia, hyperopia, astigmatism);

Treatment of keratoconus (KCN);

Little additional optical power required, resulting in substantially no cross blurring; or

Other surgeries, including additional ELP adjustment treatments, are not precluded.

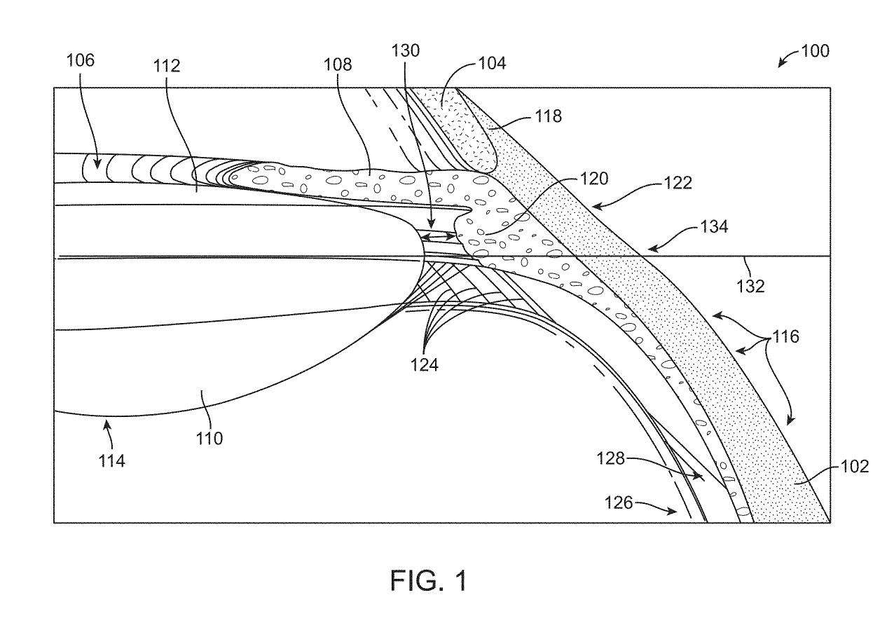

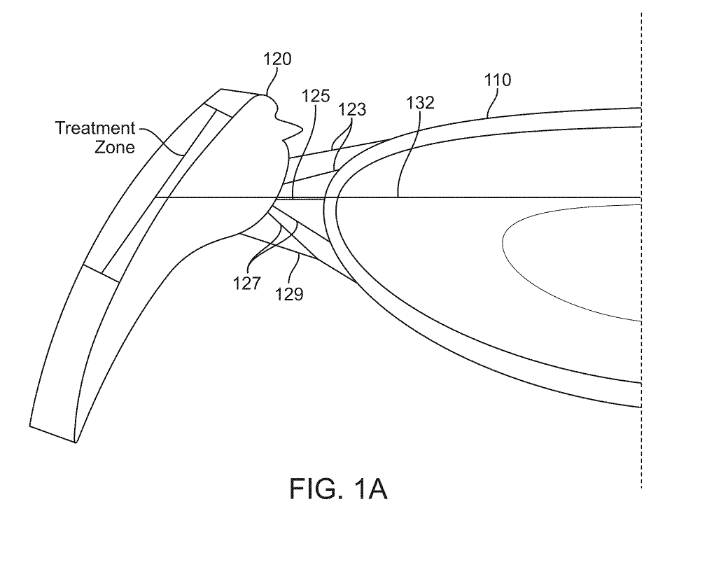

FIG. 1 illustrates a presbyopic eye 100 in a configuration for far vision, in accordance with embodiments. The eye 100 includes a sclera 102, a cornea 104, a pupil 106, an iris 108, and a lens 110 within a lens capsule, the lens capsule including a lens capsule anterior surface 112 and a lens capsule posterior surface 114. The sclera is lined by a conjunctiva 116 and includes a sclera spur 118 adjacent the cornea 104. A ciliary body 120 is adjacent the ciliary body sclera region 122. The ciliary body 120 is connected to the lens 110 by vitreal zonules 124 and to the ora serrata 126 by the posterior vitreal zonules 128 (hereinafter “PVZ”). A circumlental space 130 (hereinafter “CLS”) is defined by the distance between the lens 110 and the ciliary body 120 along a lens equator plane 132, the lens equator plane 132 passing through an equatorial sclera region 134.

FIG. 1A shows zonules of the eye 100 near the lens equator plane 132 of lens 110, and a treatment zone of the sclera 102 suitable for adjusting the effective lens position as described herein. The lens is coupled to the ciliary body 120 with several zonules, such as the anterior zonules 123, equatorial zonules 125 and posterior zonules 127. The eye also comprises an anterior hyaloid membrane 129. The effective lens position can be shifted by selectively shrinking collagen near the ciliary body 120 in order to move the ciliary body and the corresponding locations at which the zonules attach to the ciliary body, resulting in changes to the location of the lens. The treatment location can be anterior or posterior to the lens equator as described herein.

The anterior zonulae scleral insertion generally corresponds to the location of the insertion of anterior zonules 123 in to the ciliary body 120, and the location of the sclera 102 immediately exterior to this location. The posterior zonulae scleral insertion generally corresponds to the location of the insertion of posterior zonules 127 in to the ciliary body 120, and the location of the sclera 102 immediately exterior to this location. The treatment zone may extend anteriorly or posteriorly to this region, by about a 1 mm, for example. Alternatively or in combination, the treatment zone may extend into the cornea, for example.

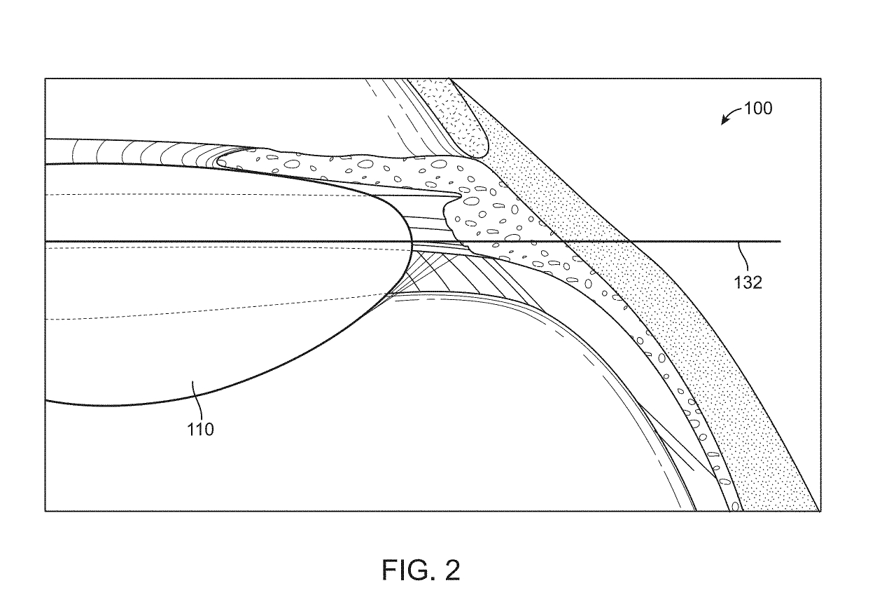

FIG. 2 illustrates the presbyopic eye 100 of FIG. 1 attempting to correct for near vision, in accordance with embodiments. In the presbyopic eye 100, the curvature of the lens 110 does not change substantially from the curvature in the far vision configuration, and the accommodative amplitude of the lens 110 along the lens equator plane 132 is relatively small.

Table 3 shows PVZ mobility and CLS size in non-presbyopic and presbyopic eyes during an unaccommodative state (“UN-ACC”) and an accommodative state (“ACC”). In non-presbyopic eyes, the length of the PVZ changes from 4.6 mm in the un-accommodative state to 3.6 mm in the accommodative state, for a net change of 1 mm. In contrast, PVZ mobility is substantially reduced in presbyopic eyes: the PVZ length changes from 4.6 mm in the un-accommodative state to 4.45 mm in the accommodative state, for a net change of only 0.15 mm. Additionally, the size of the CLS is significantly smaller in presbyopic eyes compared to non-presbyopic eyes, with measured values of 0.68 mm and 0.35 mm in the un-accommodative state, and 0.68 mm and 0.2 mm in the accommodative state, respectively.

TABLE 3
PVZ mobility and CLS size in non-
presbyopic and presbyopic eyes.

| | Non-Presbyopic | | | Presbyopic | | |
|---|---|---|---|---|---|---|
| | UN-ACC | ACC | Change | UN-ACC | ACC | Change |
| PVZ (mm) | 4.6 | 3.6 | 1 | 4.6 | 4.45 | 0.15 |
| CLS (mm) | 0.68 | 0.68 | 0 | 0.35 | 0.2 | 0.15 |

Without being bound to any particular theory, it is believed that accommodative anterior and inward ciliary apex movement is hindered by PVZ immobility in the presbyopic eye. The embodiments disclosed herein can provide improved mobility of the accommodative anterior and inward ciliary apex movement with softening of the scleral and corneal tissue as disclosed herein. The embodiments disclosed herein can provide compensation for antero-posterior lens growth, equatorial-apex position and zonular insertion angle changes, and loss in corneal elasticity with age. The embodiments disclosed herein can provide increased curvature of the lens with decreased zonular tension in order to provide increased accommodation. In many embodiments, the simultaneous expansion of the perilenticular space and softened and/or plasticized mid-scleral stroma near the ciliary body and PVZ as described herein can provide for stable distance vision (e.g., augmented by cross-linking) and restoration (e.g., an increase) of accommodative amplitude.

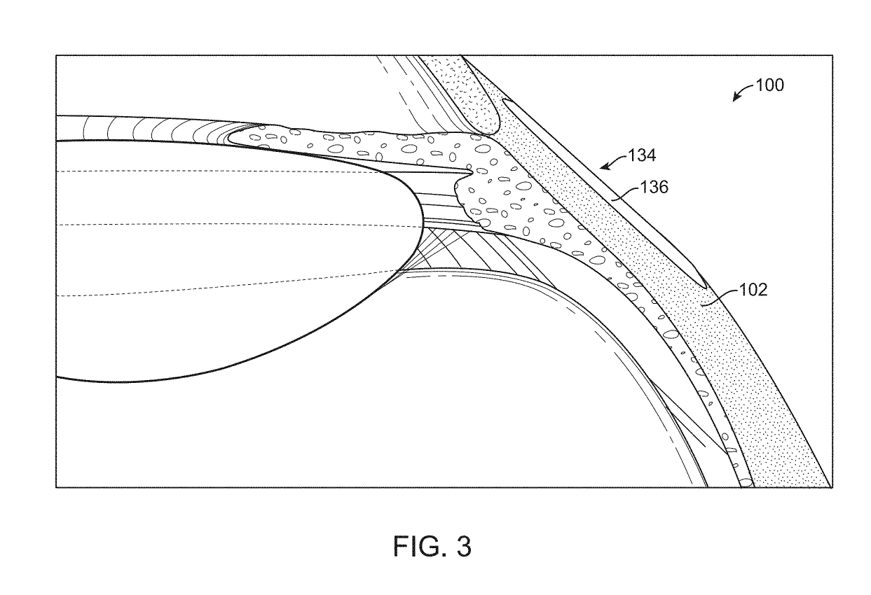

FIG. 3 illustrates stabilization of an eye 100 by cross-linking to treat presbyopia, in accordance with embodiments. The stabilized region 136 can be disposed in the outer portion of equatorial sclera region 134 of the sclera 102. Any suitable stabilization method, such as collagen cross-linking, can be used to stabilize the cross-linked region 136 in order to substantially maintain the outer profile of the sclera 102. In many embodiments, a cross-linking agent is applied to the sclera and allowed to infuse into the sclera at stabilized region 136. An energy source can be applied to the sclera to cross-link the sclera at stabilized region 136 with the cross-linking agent. The energy source can include a microelectrode array to generate a patterned cross-linked profile on the sclera. The energy can include one or more of thermal energy, radiofrequency (hereinafter “RF”) energy, electrical energy, microwave energy, light energy, or ultrasound energy.

In many embodiments, the cross-linking agent includes one or more of many known chemical photosensitizers in the presence of oxygen. Oxygen can be delivered to the stabilized region 136 of the sclera, concurrently with the cross-linking agent or separately. The cross-linking agent can be exposed to light energy when the cross-linking agent has been provided to the tissue, in order to provide cross-linking to a target depth of tissue stabilization. The light energy may include one or more of visible light energy, ultraviolet (hereinafter “UV”) light energy, or infrared (hereinafter “IR”) light energy. Alternatively or combination, the cross-linking agent may include a chemical cross-linking agent. In many embodiments, the cross-linking agent includes one or more of the following: riboflavin, rose bengal, methylene blue, indocyanine green, genipin, threose, methylglyoxal, glyceraldehydes, aliphatic β-nitro alcohols, black currant extract, or an analog of any of the above.

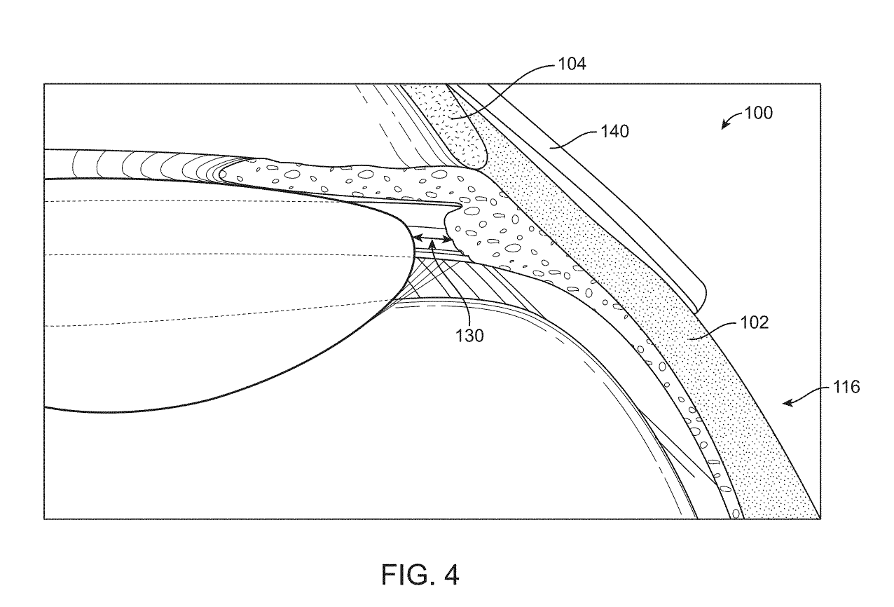

FIGS. 4-6 illustrate aspects of provide scleral translocation and elasto-modulation (hereinafter “STEM”) adjustment treatment procedure to expand the CLS and thereby enhance ciliary body apex mobility in order to increase the accommodative amplitude of the eye, suitable for combination in accordance with ELP embodiments disclosed herein. The CLS can be expanded by applying energy to shrink and/or plasticize an inner portion of the eye, such as the inner portion of the sclera (e.g., the mid-stroma), so as to move the ciliary body apex outward and thereby increase the ciliary ring diameter. In many embodiments, the outward movement includes a radially outward movement away from the optical axis of the eye and towards a stabilized outer portion of the eye (e.g., the cross-linked region 136). The energy to shrink and/or plasticize the inner portion of the eye can include one or more of thermal energy, RF energy, mechanical energy, electrical energy, microwave energy, light energy, high intensity ultrasound energy, or ultrasound energy. The energy can shrink and/or plasticize the tissue by heating the tissue to a suitable temperature without substantially weakening the tissue, such as within a range from about 50° C. to 70° C. Heating the tissue can increase the elasticity of the tissue. In many embodiments, the heat is applied such that the outer portion of the tissue remains substantially viable so as to inhibit post-operative pain and swelling. While in many embodiments the energy can be applied through the conjunctiva and/or epithelium, the energy can also be applied with the conjunctiva and/or epithelium moved away from the sclera. The energy source can be the same energy source used to cross-link the eye, as described herein, or a different energy source.

FIG. 4 illustrates a heat sink 140 placed over the eye 100 of FIG. 3 in order to treat presbyopia, in accordance with embodiments. The heat sink 140 can be inserted over an outer portion of the eye 100 including the cornea 104, sclera 102, and conjunctiva 116, in order to conduct heat away from the outer portion of the eye 100 during the treatment procedure. The heat sink can be made of any suitable material. For example, the heat sink can include a material transmissive to wavelengths of light energy (e.g., sapphire of diamond-like carbon transmissive to certain wavelengths of IR light), so that the eye tissue beneath the heat sink can be heated with absorbed light energy.

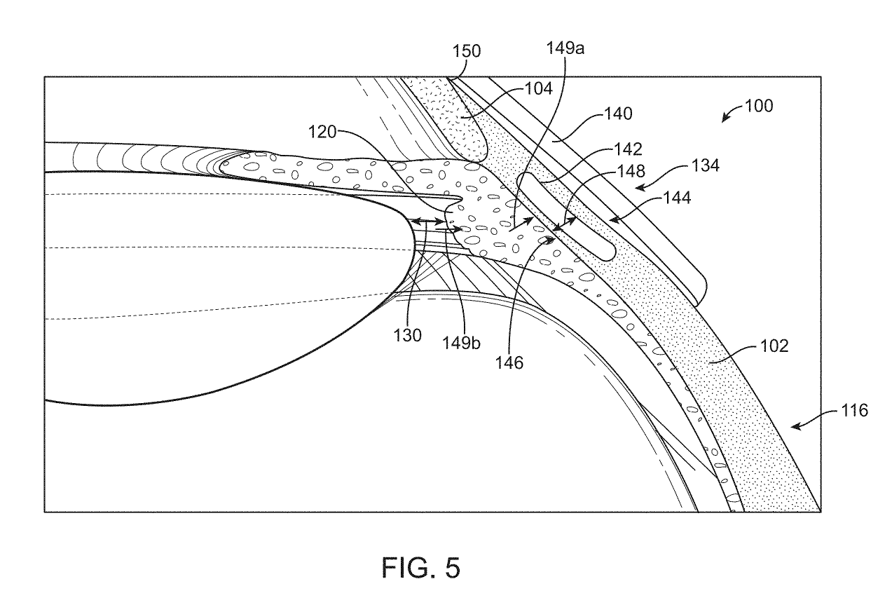

FIG. 5 illustrates a planned treatment zone 142 in the eye 100 of FIG. 4 for treating presbyopia, in accordance with embodiments. The planned treatment zone 142 can be disposed between an outer surface 144 (e.g., adjacent the conjunctiva 116) and inner surface 146 (e.g., adjacent the apex of the ciliary body 120 or a trabecular meshwork (not shown)) of the equatorial sclera region 134 of an eye 100. The equatorial sclera region 134 has an initial sclera thickness 148 defined by the distance between outer surface 144 and inner surface 146. The treatment can be applied by a laser to the treatment region 142 to heat and shrink and/or plasticize the mid-stroma of the equatorial sclera region 134, thereby causing the inner sclera surface 146 and inner ciliary body 120 to move in centrifugal directions 149a, 149b, while avoiding the conjunctiva 116 and ciliary muscles adjacent the ciliary body 120. The laser can be scanned through the sclera 102 posterior the limbus 150 such that limbal stem cells and insertions of the rectus muscles of the eye 100 are avoided.

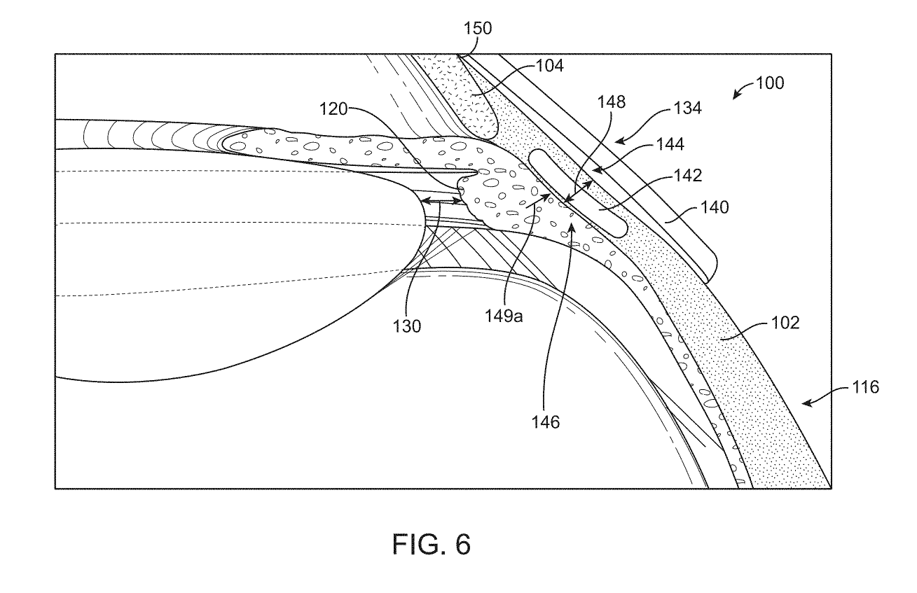

FIG. 6 illustrates laser treatment of the eye 100 of FIG. 5 to treat presbyopia, in accordance with embodiments. The laser treatment can be applied to the treatment zone 142 to shrink and/or plasticize the tissue in the treatment zone 142 and thereby expand the CLS 130. Compared to the pre-treatment eye 100 of FIG. 5, the profile of the outer sclera surface 144 is substantially maintained (e.g., by stabilization as described herein), while the profile of the inner sclera surface 146 moves in a centrifugal direction 149a and is deflected substantially outward, resulting in a decreased sclera thickness 148 of the equatorial sclera region 134. The shrinkage of the mid-stroma causes the inner profile of the ciliary body 120 to move centrifugally outward toward the outer sclera surface 144, producing an increase in the size of the CLS 130 and an enhancement in the inward mobility of the ciliary body 120 during accommodation.

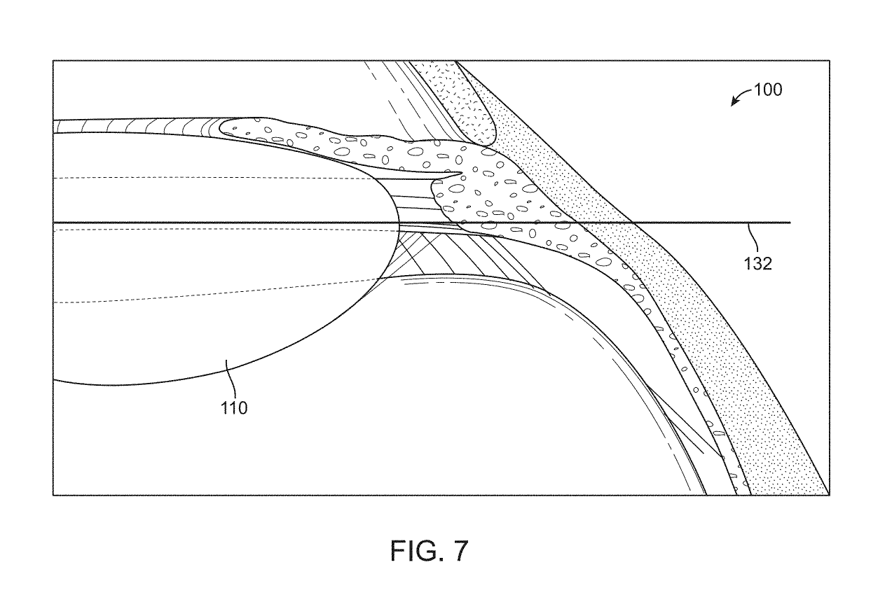

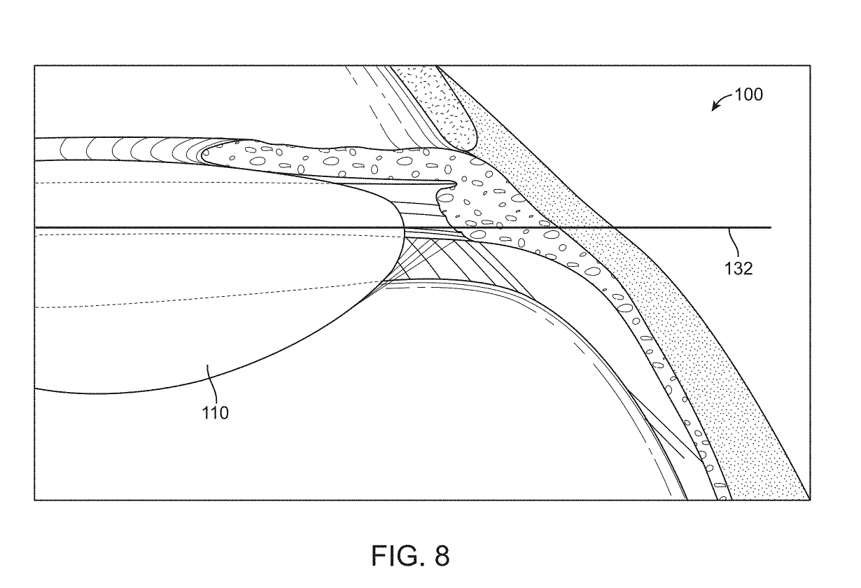

Referring to FIGS. 7 and 8, an enhancement in centrifugal accommodative and un-accommodative movement of the eye 100 of FIG. 6 is observed following CLS expansion, in accordance with embodiments. FIG. 7 illustrates the postoperative eye 100 in a near vision configuration with the lens 110 in an accommodative state. FIG. 8 illustrates the post-operative eye 100 in a far vision configuration with the lens 110 in a un-accommodative state. Mobility of the ciliary body apex has been restored, and substantial changes in the curvature of the lens 110 and large accommodative amplitude along the lens equator plane 132 are observed, in contrast to the presbyopic eye of FIGS. 1 and 2.

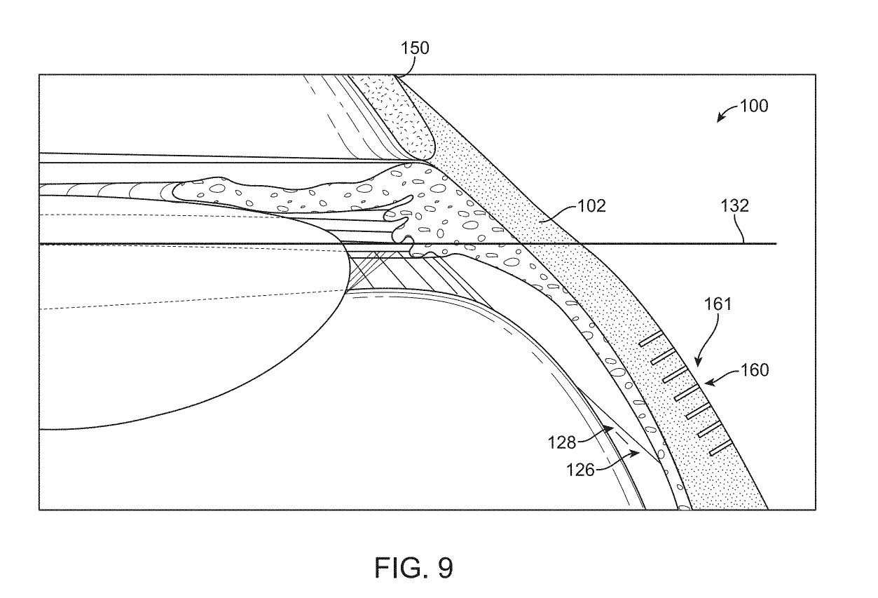

FIG. 9 illustrates treatment of the eye 100 to soften the sclera proximate the insertion location of the PVZ 128 to treat presbyopia, in accordance with embodiments. The treatment region can extend posterior to the lens equator plane 132 and anterior to the insertion location of the PVZ 128 at the ora serrata 126. The treatment can be applied to the treatment region to ablate the tissue and form tiny fenestrations 160 within a scleral softening region 161 of the sclera 102. Alternatively or in combination, the tissue can be softened without ablation. In many embodiments, the PVZ insertion location can be softened order to enhance mobility of the PVZ and thereby increase the anterior mobility of the ciliary body apex during accommodation. Any suitable method can be used, such as laser-induced softening and/or plasticizing, to soften and/or plasticize any suitable portion of the sclera. The softening can include heating the portion of the sclera to a suitable temperature to weaken the tissue, such as within a range from about 70° C. to 90° C. The heat can be applied using energy, such as one or more of thermal energy, RF energy, mechanical energy, electrical energy, microwave energy, light energy, high intensity focused ultrasound energy, or ultrasound energy. The energy may be emitted by the same energy source used to cross-link the eye or shrink and/or plasticize the inner portion of the eye, or by a different energy source. The softening and/or plasticizing treatment can be applied at any suitable location such that damage to non-treatment regions of the eye, such as muscles of the eye, is avoided. For example, the treatment can be applied to soften and/or plasticize four portions of the sclera, each corresponding to a location away from muscles of the eye including inferior muscles, superior muscles, nasal muscles, and temporal muscles. In many embodiments, after softening and/or plasticizing, the mobility of the PVZ 128 in accommodated and un-accommodated states is enhanced, and the anterior movement of the ciliary body apex is restored.

FIG. 10 illustrates aspects of STEM treatment procedure to enhance corneal bending of the eye to treat presbyopia, or glaucoma, or both, suitable for combination in accordance with ELP embodiments disclosed herein. In many embodiments, inner portions of the scleral spur and/or the cornea lateral to the Schlemm's canal and trabecular meshwork can be heated to increase the elasticity of the eye near the scleral spur inner portions, thereby enhancing corneal bending during accommodation to treat presbyopia, for example. For example, energy can be applied to shrink and/or plasticize the inner portions by heating the tissue to a suitable temperature without substantially weakening the tissue, such as within a range from about 50° C. to 70° C. Alternatively, energy can be applied to soften the inner portions by heating the tissue to a suitable temperature to weaken the tissue, such as within a range from about 70° C. to 90° C. Any suitable energy source can be used to enhance corneal bending, as described herein. The energy source can be the same energy source used to cross-link the eye or soften the PVZ insertion location, as described herein, or a different energy source, for example.

FIG. 10 illustrates a planned treatment of the eye 100 of FIG. 9 to soften the tissue lateral to the Schlemm's canal and trabecular meshwork to treat presbyopia, in accordance with embodiments. The Schlemm's canal 170 and trabecular meshwork 172 are positioned within the inner portion of the cornea 104 adjacent to the scleral spur 118 of the sclera 102. A planned treatment zone 174 can be disposed within the cornea 104 lateral to the Schlemm's canal 170, trabecular meshwork 172, and scleral spur 118. In many embodiments, the treatment zone 174 can be located outside the optically used portion of the cornea 104 (e.g., the peripheral corneal stroma). Alternatively or in combination, the treatment zone 174 can be located within a portion of the sclera 102 lateral to the Schlemm's canal 170 and trabecular meshwork 172, such as the scleral spur 118. The outer portion of the cornea 104 and/or the scleral spur 118 lateral to the planned treatment zone 174 can be cross-linked to create a stabilized outer profile, as previously described herein.

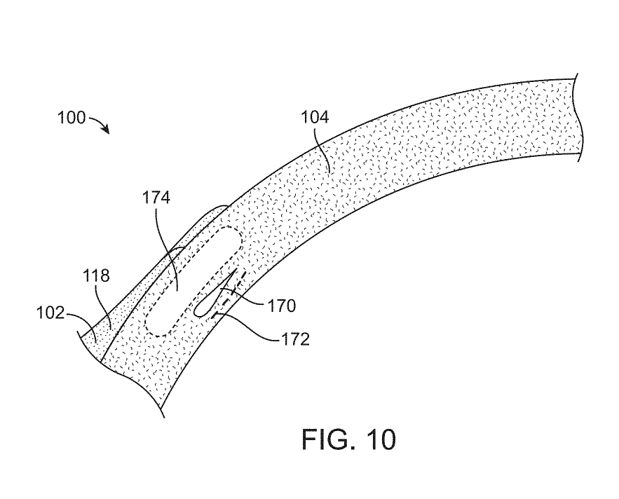

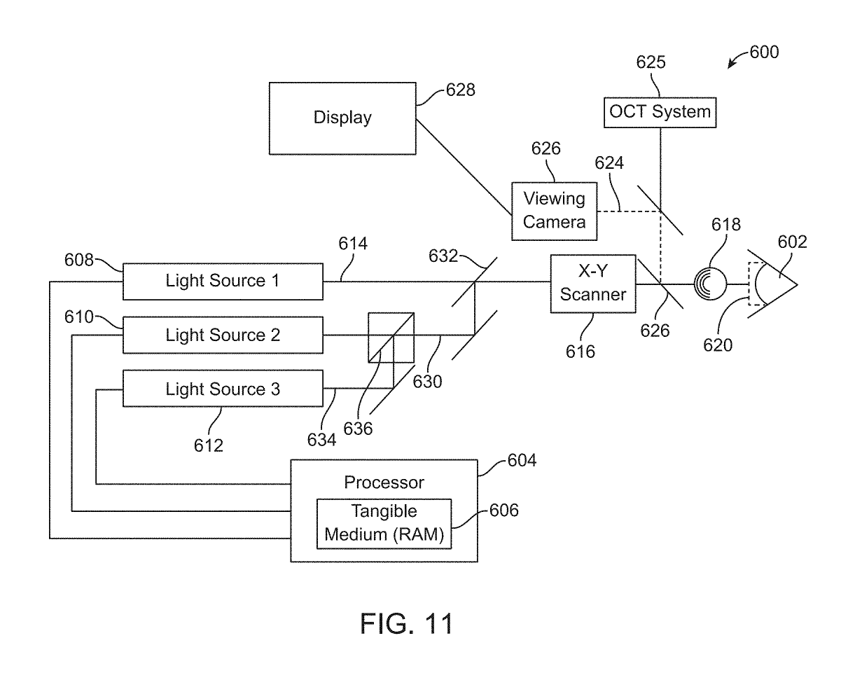

FIG. 11 illustrates a system 600 for treating an eye 602, in accordance with embodiments. The system 600 includes a processor 604 having a tangible medium 606 (e.g., a RAM). The processor 604 is operatively coupled to a first light source 608, a second light source 610, and a third light source 612. The first light source 608 emits a first beam of light 614 that is scanned by X-Y scanner 616 through an optional mask 618 and optional heat sink 620 onto the eye 602. The mirror 622 directs light energy from the eye 602 to a viewing camera 627 coupled to a display 628. An independent non-treatment light source for the viewing camera can be provided, for example. The mirror 622 may direct a portion of the light beam returning from eye 602 to the camera 627, for example. The second light source 610 emits a second beam of light 630 that is combined by a first beam combiner 632 with the first beam of light 614 prior to passing through X-Y scanner 616. The third light source 612 emits a third beam of light 634 that is combined by a second beam combiner 636 with the second beam of light 630 prior to passing through the first beam combiner 632.

The processor may be configured with instructions to determine the effective position or orientation (e.g. ELP) of the lens of the eye. The processor may determine a direction of movement or rotation of the lens toward the ELP. In response to the direction of movement or rotation of the lens, the processor may deliver one or more of shrinkage energy to a region of the sclera to shrink the sclera or relaxation energy to a region of the sclera to relax the sclera. Shrinkage energy may for example be delivered to a first region of the sclera to shrink the sclera and relaxation energy may be delivered to a second region of the sclera to relax the sclera in response to the determined direction of movement or rotation of the lens toward the ELP.

The optical delivery system may comprise one or more of the first light source, second light source, third light source, X-Y scanner, optional mask, or a heat sink. The energy may be directed by the optical energy delivery system to the eye or a hand held probe.

In many embodiments, the beams of light 614, 630, and 634 can be scanned onto the eye 602 at a specified X and Y position by the X-Y scanner 616 to treat the eye 602. The X-Y scanner can be configured to scan the combined light beams onto the eye 602 in a suitable treatment scan pattern, as previously described herein. An optional mask 618 can be used to mask the light applied to the eye 602, for example, to protect masked portions of the eye 602 while treating other portions as described herein. An optional heat sink 620 can be placed on the eye 602 during treatment to avoid heating specified portions of the eye 602, as described herein.

The system 600 can be used to apply light energy to the eye 602 in accordance with any suitable treatment procedure, such as the embodiments described herein. In many embodiments, the first light beam 614 has a first wavelength, the second light beam 630 has a second wavelength, and the third light beam 634 has a third wavelength. Each wavelength can be a different wavelength of light. Alternatively, at least some of the wavelengths can be the same. For example, in accordance with the embodiments described herein, the first light beam 614 can have a wavelength suitable to: cross-link an outer portion of the eye 602 and shrink an inner portion of the eye 602; shrink the inner portion and cross-link the outer portion concurrently; shrink the inner portion after the outer portion has been cross-linked; or any suitable combinations thereof. Alternatively, the first light beam 614 can have a first wavelength suitable to cross-link the outer portion of the eye 602, as described herein, and the second light beam 630 can have a second wavelength suitable to shrink the inner portion of the eye 602, as described herein. The third light beam 634 can have a third wavelength suitable to soften a portion of the sclera of the eye 602, as described herein. Any suitable combination of wavelengths of light for applying any combination of the treatments described herein, concurrently or separately, can be used.

The processor can be coupled to each of the light sources in order to selectively irradiate the eye with light having wavelengths within a desired range of wavelengths. For example, the first light source can be configured to emit light energy having wavelengths in a range from about 1.9 to 2.1 um, the 1/e attenuation depth can be in a range from about 200 to 300 ums, for example about 225 to 275 um. The second light source can be configured to emit light energy having wavelength in a range from about 1.3 to 1.55 um, the 1/e attenuation depth is within a range from about 350 to 450 um. The processor can be programmed with instructions to irradiate tissue with light energy appropriate for the effect at the desired treatment location. For example, the light source emitting light energy in the range from 1.9 to 2.1 um can be used to treat the cornea, and the second light source emitting light energy with wavelengths in the range from 1.3 to 1.55 um can be used to irradiate the sclera. The software may comprise instructions of a treatment table so as to scan the laser beam to desired treatment locations as described herein, for example.

The laser system 600 may comprise an OCT system 625, such as a commercially available OCT system. The OCT system may for example be a CASIA2 or CASIA SS-100 OCT scanner (TOMEY). The OCT system can be coupled to the viewing optics and laser delivery system with a beam splitter 626. The OCT system can be used to measure the eye in situ during treatment. For example, the OCT system can be used to generate OCT images as described herein in order to generate tomography of the eye to determine the location of target tissues, movement of target tissues and stretching of target tissues as described herein. The OCT system 625 can be coupled to processor 604 and used to control the laser system with a feedback loop, for example.

The processor can be configured with instructions to scan the laser beam on the eye in accordance with the treatment patterns and parameters as described herein.

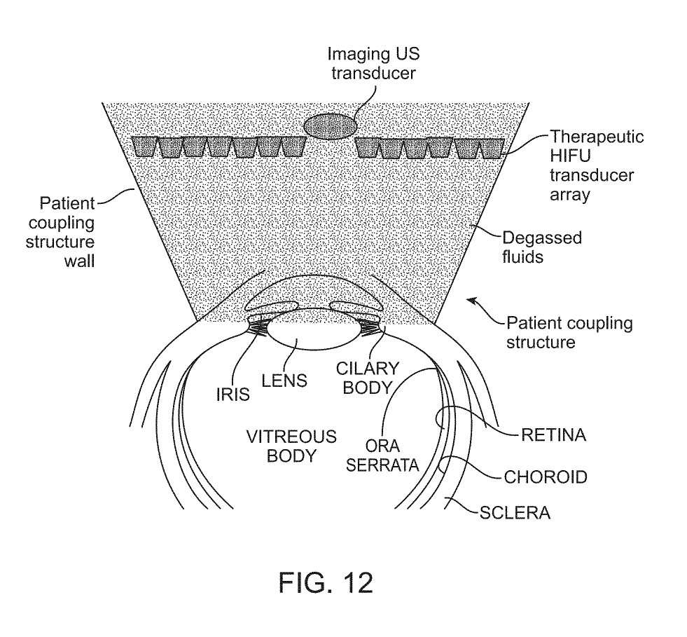

FIG. 12 shows a treatment setup for ELP adjustment using ultrasound, for example high intensity focused ultrasound (HIFU), as an energy source. In one embodiment, a therapeutic HIFU transducer array which comprises a centrally positioned imaging system, for example an ultrasound transducer or OCT fiber, may be coupled to the eye with a patient coupling structure comprising a conic-shaped wall and a degassed fluid therein. The fluid interface may serve as a space to tightly focus the ultrasound beam to the desired treatment zone. Alternatively or in combination, the fluid may allow for greater control and/or a greater range over depth from the tissue surface. The fluid may further be used to control the temperature of surface tissue during exposure to HIFU during treatment. The fluid is preferably chilled to about 4° C. and may be one or more of a gel, a gel pack, water or trehalose. The HIFU array may be aimed at a cavitational zone appropriate for the ELP adjustment treatment area.

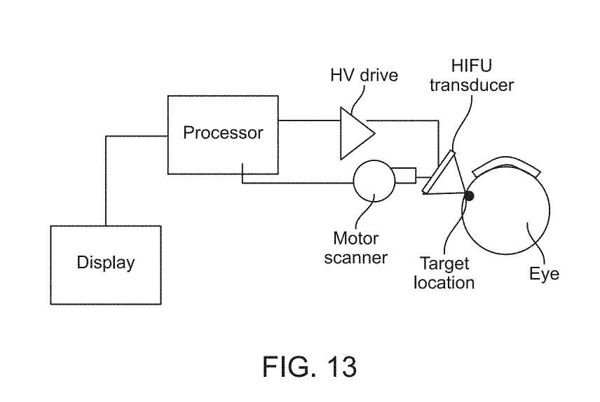

FIG. 13 shows an ELP adjustment system comprising HIFU. The system comprises a HIFU array focusing ultrasound energy to a location inside the eye. A motor scanner can optionally be coupled to the ultrasound array to direct the treatment energy to the target locations of the eye. A processor may be coupled to a high voltage drive (HV) to drive the array. The processor can be coupled to the motor scanner to move the array during treatment. A display can be coupled to the processor to show an image of the eye. The image of the eye can be generated with imaging frequencies and wavelengths and the HIFU can be delivered to the eye with HIFU wavelengths as described herein.

One or more of the motor scanner, HIFU transducer, patient coupling structure, and imaging system may comprise an energy delivery system. The energy may be directed by the ultrasound energy delivery system to the eye or a hand held probe.

The processor may be configured with instructions to determine the effective position or orientation (e.g. ELP) of the lens of the eye. The processor may determine a direction of movement or rotation of the lens toward the ELP. In response to the direction of movement or rotation of the lens, the processor may deliver one or more of shrinkage energy to a region of the sclera to shrink the sclera or relaxation energy to a region of the sclera to relax the sclera. Shrinkage energy may for example be delivered to a first region of the sclera to shrink the sclera and relaxation energy may be delivered to a second region of the sclera to relax the sclera in response to the determined direction of movement or rotation of the lens toward the ELP.

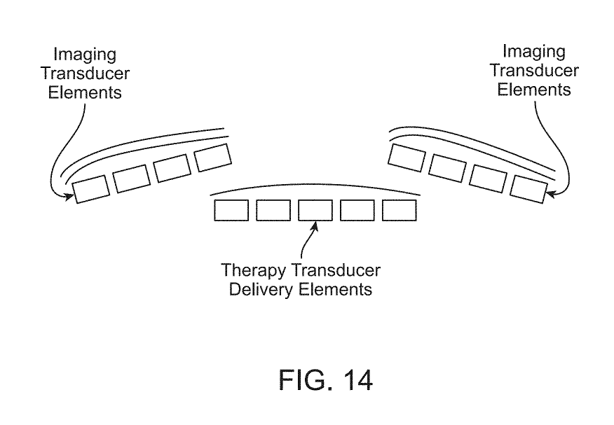

FIG. 14 shows a HIFU array coupled to an imaging apparatus. A pair of ultrasound imaging arrays and a HIFU array are arranged for real-time imaging during treatment. The imaging transducer elements and therapy transducer and elements can be coupled to the processor as disclosed herein. Coupling the imaging apparatus to the HIFU transducer allows for passive cavitation detection and imaging feedback to guide and inform treatment.

The HIFU transducer may comprise one or more of a phased array, a discrete array, an annular array, a spherical array, a spherical phased array, or any combination thereof. The HIFU transducer may be combined with an imaging apparatus, for example embedded OCT sensors. Additionally, the transducer may be fabricated to allow for opto-acoustic excitation for precise theranostic delivery.

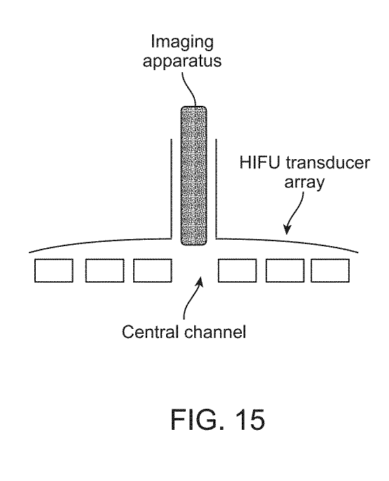

FIG. 15 shows another embodiment of a HIFU array coupled to an imaging apparatus. The HIFU array in this embodiment comprises a transducer with central channel in which the imaging apparatus may be disposed. For example, the imaging apparatus may be an OCT fiber optic cable. The OCT fiber may be disposed inside a channel extending from the center of the therapy transducer and can allow for real-time imaging of tissue at one or more times before, during, or after treatment with the HIFU array.

The HIFU array may be coupled to a number of imaging systems, including but not limited to MRI, UBM, ultrasound imaging, OCT, OCE, or US elastography.

Each of the HIFU arrays and imaging apparatus shown in FIGS. 12-15 can be similarly coupled to a processor and display for controlling treatment, for example as shown with reference to FIG. 13, and a person of ordinary skill in the art will understand how to construct such as system in accordance with the teachings disclosed herein. Further, for each of the laser treatments as disclosed herein, a person of ordinary skill in the art can construct a HIFU system to treat regions of the eye similarly with heat to obtain similar treatment outcomes.

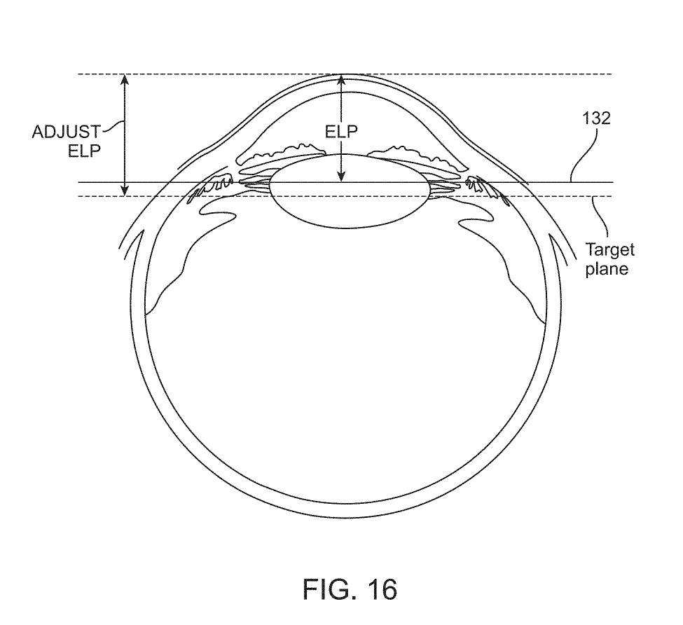

FIG. 16 shows ELP adjustment. The ELP adjustment system described herein may be used to delivery energy to induce equatorial scleral translocation posterior and anterior the target plane in order to adjust the ELP of a lens such as an IOL after insertion toward the target plane. The effective lens position may comprise a distance from the vertex of the cornea to the lens equator plane 132. The effective lens position (ELP) can be adjusted to correct refractive error of the eye with energy as described herein. Decreasing the distance from the lens equator to the vertex of the cornea will make the eye more near sighted, while increasing the ELP will make the eye more far sighted. This approach can be used to correct refractive error of the eye. With a myopic (“near sighted”) eye, the lens of the eye can be moved posteriorly to decrease the optical power of the eye and allow the eye to see far objects, thereby correcting the refractive error of the eye. With a hyperopic (“far sighted”) eye, the lens can be moved anteriorly to increase the optical power of the eye, and allow the eye to see distance objects in focus. For an astigmatic eye, the lens of the eye can be tilted so as to correct astigmatism of the eye. The corrected astigmatism may comprise corneal astigmatism, in which induced tilt of the lens corrects the corneal astigmatism. The corrected astigmatism may comprise lenticular astigmatism, in which changing the angle of the lens can correct the lenticular astigmatism. The treatment methods and apparatus as disclosed herein can be used shrink or relax tissue, in order to adjust the effective lens position with treatment to the sclera or the cornea, and combinations thereof. The treatment can be combined with chilling of the lens in order to maintain viability of the exterior layers of the eye as described herein. Although reference is made to movement of the lens equator, the reference location of the lens may comprise one or more nodes of the lens, and the treatment determined in order to move a node of the lens of the eye to a target location to correct vision.

A more anterior ELP corresponds to a lower IOL power while a more posterior ELP corresponds to a higher IOL power required for the same prescription. The ELP adjustment system may be used in mechanical mode to generate balanced fractionation or shrinkage along the sclera for 360 degrees about the target plane resulting in stretching or yielding of the sclera and ELP adjustment, for example with ultrasound. Alternatively or in combination the ELP adjustment system may be operated in thermal mode to induce annular shrinkage of the sclera, for example with thermal energy such as ultrasound, laser and other thermal sources of energy as described herein. ELP adjustment may be done after cataract surgery or LASIK for touchups for example. Post-operative ELP errors may for example occur due to IOL shifts, pre-operative calibration calculation errors, capsular shrinkage, extra-lenticular realignment, or any combination thereof. The ELP treatment can be performed on a natural lens of an eye, such as a presbyopic or non-presbyopic natural lens, for example to treat myopia, hyperopia, astigmatism and any combination thereof.

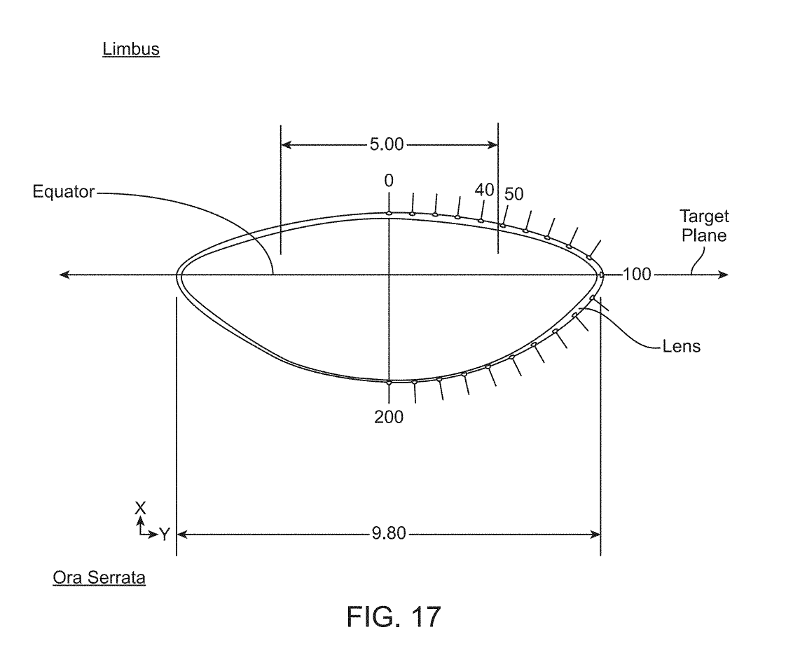

ELP adjustment may for example include translation, tilting, centering, or any combination thereof due to regional scleral shrinkage (elastification) or relaxation (compliance) about a line along a target plane. ELP adjustment of about 1 mm may correspond to a focal change of about 2.0 D for example. FIG. 17 shows a lens with a normal ELP. The lens lies along the target plane such that a line along the target plane passes through the equator of the lens and the lens in optimally situated between the limbus and the ora serrata.

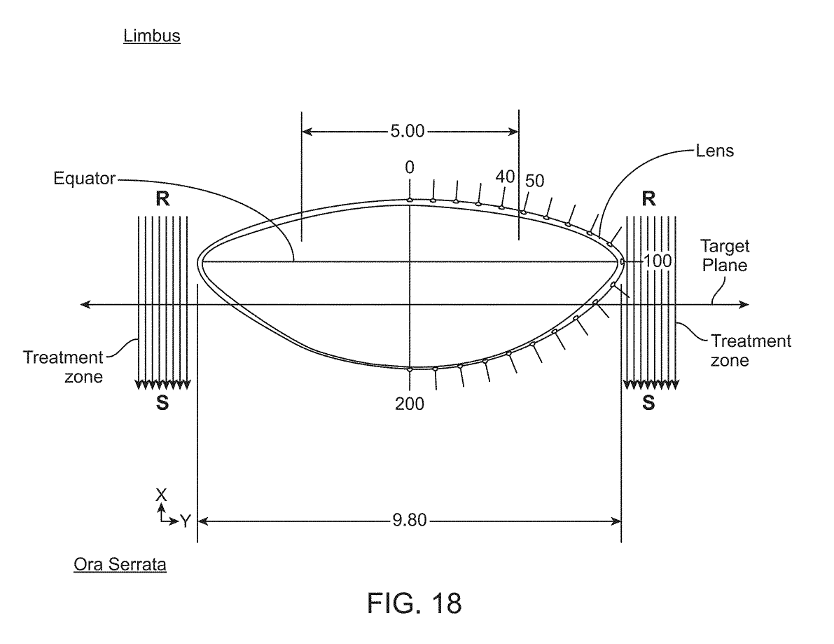

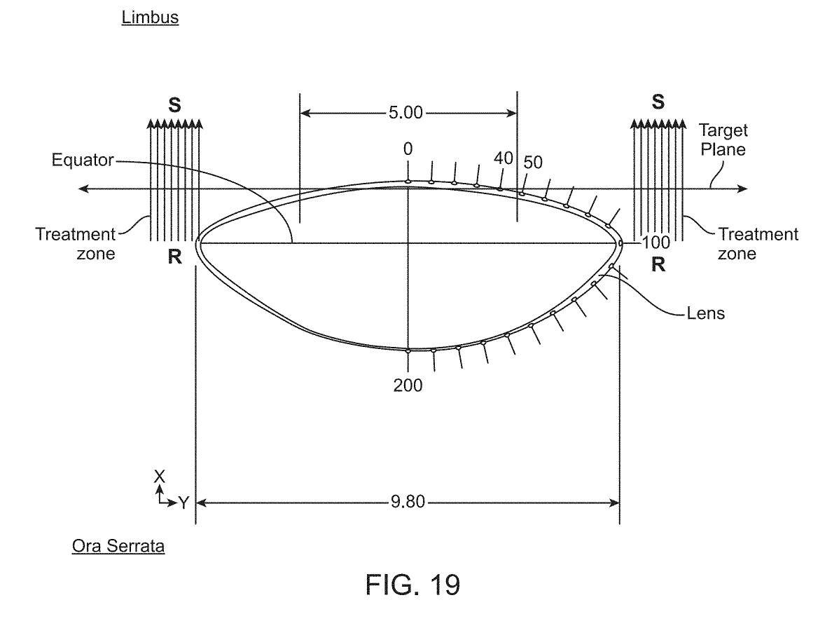

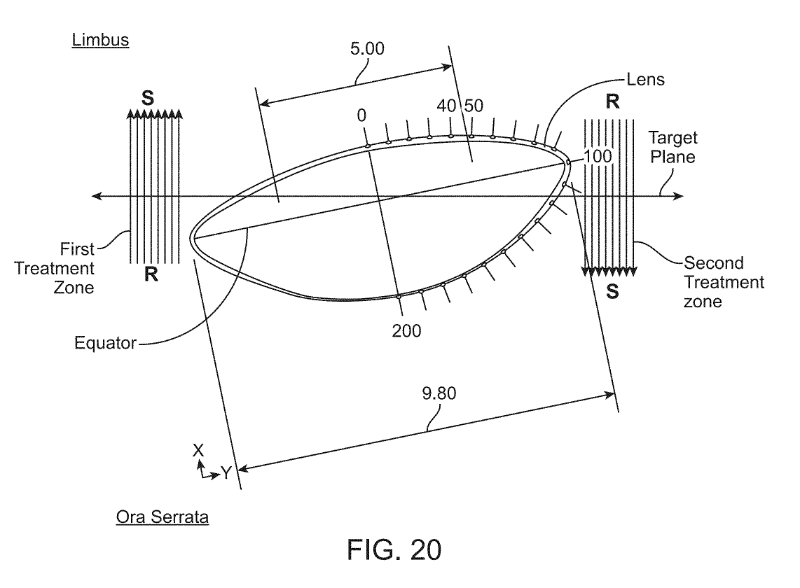

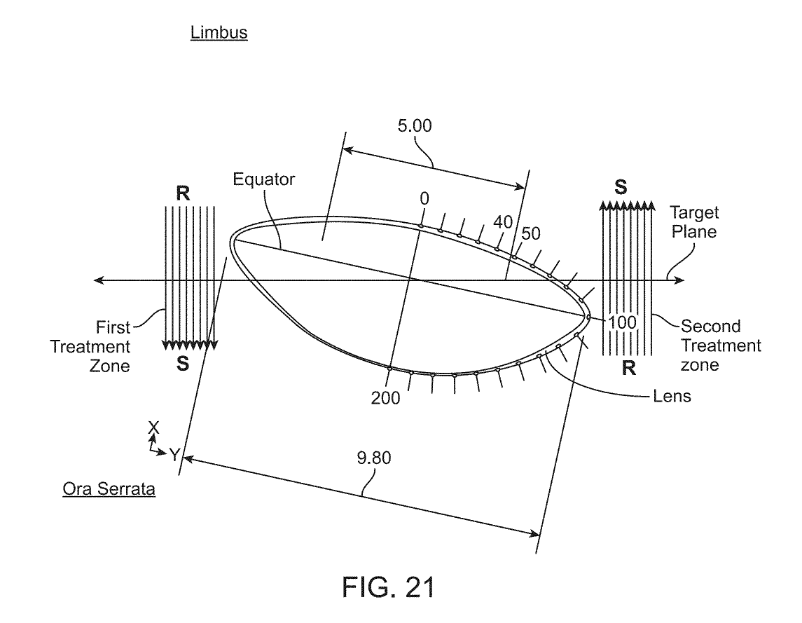

FIGS. 18-21 show additional embodiments of treatment zones for ELP adjustment. Treatment may be focused to a region extending from about 1 mm anterior to the anterior equatorial zonulae scleral insertion to about 1 mm posterior to the posterior equatorial zonulae scleral insertion for 360 degrees. Scleral shrinkage S may be induced on the side of the target plane further from the lens equator while scleral relaxation R may be induced on the side of the target plane closest to the lens equator so as to adjust the lens equator towards the target plane. ELP adjustment using the ELP adjustment system described herein may occur without changing axial length or corneal curvature. FIG. 18 shows a treatment zone for ELP adjustment. The lens is myopically shifted such that the lens equator is located above the target plane. The ELP adjustment system described herein may be used to induce scleral shrinkage anterior to the lens target plane as well as scleral relaxation posterior to the lens target plane within the treatment zone to return the lens to the position described in FIG. 17. FIG. 19 shows another embodiment of a treatment zone for ELP adjustment. In this embodiment, the lens is hyperopically shifted such that the lens equator is located below the target plane. The ELP adjustment system described herein may be used to induce scleral relaxation anterior to the lens target plane as well as scleral shrinkage posterior to the lens target plane within the treatment zone to return the lens to the position described in FIG. 17. FIG. 20 shows yet another embodiment of a treatment zone for ELP adjustment. In this embodiment, the equator of the lens is at an angle relative to the target plane. The ELP adjustment system described herein may be used to adjust the ELP by adjusting the effective lens angle (ELA) of the lens. The system may be used to induce arcuate shrinkage and relaxation about the target plane in the first treatment zone and 180 degree mirrored arcuate relaxation and shrinkage about the target plane in the second treatment zone in order to rotate the lens towards the shrinkage and away from the relaxation to return the lens to the position described in FIG. 17. A treatment zone for ELA adjustment may for example have a distinct symmetric orthogonal bowtie pair pattern with meridional degree-based shrinkage and relaxation. FIG. 21 shows an additional embodiment of a treatment zone for ELP adjustment. In this embodiment, the equator of the lens is at a different angle relative to the target plane and the lens angle in FIG. 20. The ELP adjustment system described herein may be used to adjust the ELA of the lens by inducing arcuate relaxation and shrinkage zone about the target plane in the first treatment and 180 degree mirrored arcuate shrinkage and relaxation about the target plane in the second treatment zone in order to rotate the lens toward the shrinkage region and away from the relaxation region and return the lens to the position described in FIG. 17. Such symmetrical ELA adjustments as described herein may for example be used to treat astigmatism or for coma adjustment.

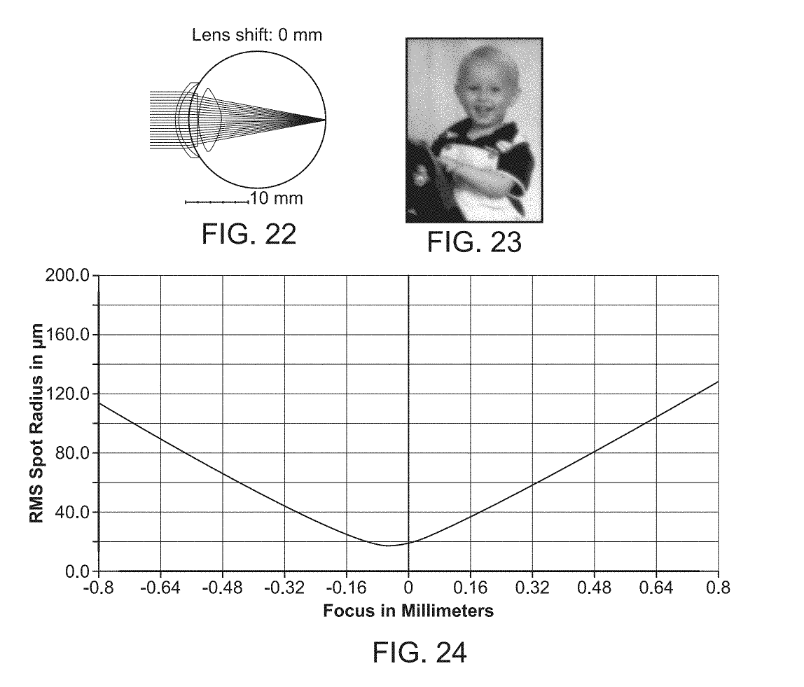

FIGS. 22-51 show ZEMAX simulations of the effects of ELP adjustment using the ELP adjustment system described herein to induce scleral cavitation. The optical ray tracing software ZEMAX was used to simulate a standard human eye (using the Gullstrand model) and the imaging behavior of the eye in response to changes of the position of the crystalline lens. The focusing condition on the retina, spot sizes on the retina, focal shift, and image quality of the eye were simulated at various ELP positions.

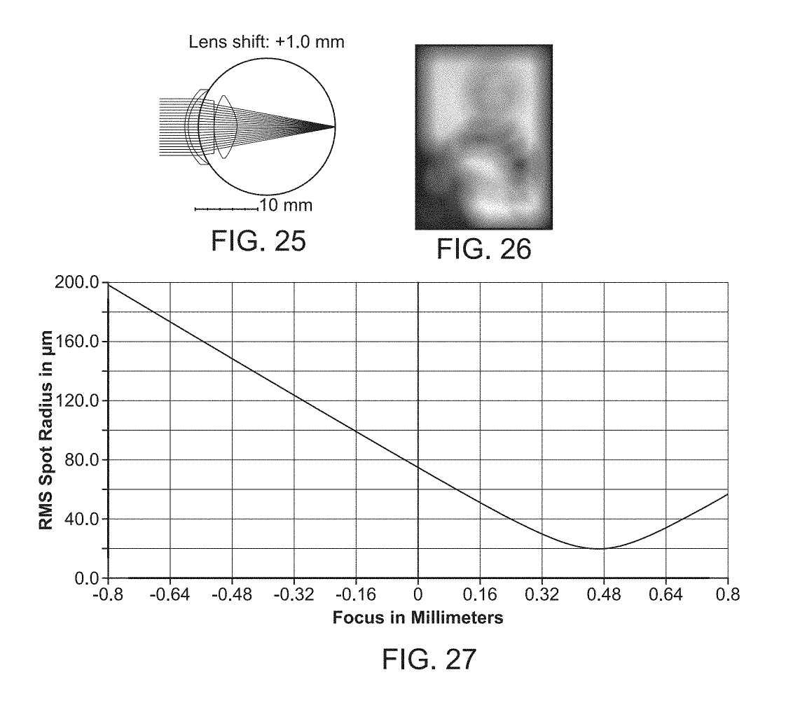

FIGS. 22-30 show simulated ocular lenses in several configurations with axial displacement. The model eye was adapted to image an object at 0.5 m distance onto the retina prior to performing the simulations. Shown here are simulations of a model eye (FIGS. 22, 25, and 28), a simulated image of a boy on the retina (FIGS. 23, 26, and 29), and the root mean square (RMS) value for best focal position onto the retina (FIGS. 24, 27, and 30) of each of the three different configurations. FIGS. 22-24 show a simulated ocular lens without any axial displacement. FIGS. 25-27 show a simulated ocular lens with +1.0 mm axial displacement. This simulation resulted in a focal shift on the retina of 0.51 mm. Furthermore, the simulation found that the object would need to be moved from 0.5 m to 2.114 m to achieve a focused image on the retina, resulting in an object displacement, or refractive change, result of 1.53 D. The refractive index change was calculated using the following formula:

$$RC = \frac{1}{{d}_{0}} - \frac{1}{{d}_{1}} \qquad ( 2 )$$

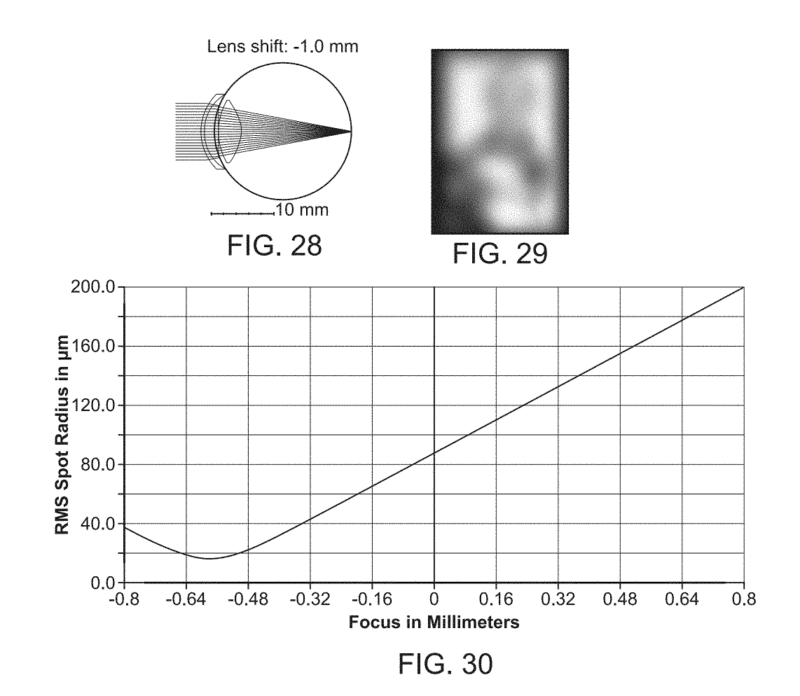

with the desired object distance d₀=0.5 m and real object distance at displaced crystalline lens, d₁=2.114 m. FIGS. 28-30 show a simulated ocular lens with −1.0 axial displacement. The simulation resulted in a focal shift of 0.52 mm. In order to achieve a focused image on the retina, the simulation found that the object would need to be move to a distance of 0.28 m, resulting in a refractive change of 1.58 D.

Additional simulations were performed with the model eye adapted to image an object at 0.4 m, 0.7 m, and 4 m distance onto the retina prior to performing the simulations. The simulated ocular lens was shifted from −1.0 mm to +1.0 mm axial displacement in 0.5 mm increments. The focal shift on the retina and the object distance to achieve a focused image on the retina were simulated. The resulting refractive change was calculated at each axial lens position using Formula 2.

Table 4 shows the results of the simulated effect of axial crystalline lens displacement for an object at 0.4 m distance.

| Axial | Focal | Object Distance | Refractive |
|---|---|---|---|
| Displacement | Shift | for Focus | Change |
| (mm) | (mm) | (m) | (D) |
| −1.0 | −0.54 | 0.244 | −1.60 |
| −0.5 | −0.27 | 0.303 | −0.80 |
| 0 | 0 | 0.4 | 0 |
| 0.5 | 0.25 | 0.581 | 0.78 |
| 1.0 | 0.52 | 1.055 | 1.55 |

Table 5 shows the results of the simulated effect of axial crystalline lens displacement for an object at 0.7 m distance.

| Axial | Focal | Object Distance | Refractive |
|---|---|---|---|
| Displacement | Shift | for Focus | Change |
| (mm) | (mm) | (m) | (D) |
| −1.0 | −0.516 | 0.34 | −1.51 |
| −0.5 | −0.249 | 0.46 | −0.75 |
| 0 | 0 | 0.7 | 0 |
| 0.5 | 0.246 | 1.434 | 0.73 |
| 1.0 | 0.49 | ∞ | 1.428 |

Table 6 shows the results of the simulated effect of axial crystalline lens displacement for an object at 4 m distance.

| Axial | Focal | Object Distance | Refractive |
|---|---|---|---|
| Displacement | Shift | for Focus | Change |
| (mm) | (mm) | (m) | (D) |
| −1.0 | −0.49 | 0.611 | −1.39 |
| −0.5 | −0.24 | 1.066 | −0.69 |
| 0 | 0 | 4 | 0 |
| 0.5 | 0.24 | ∞ | 0.25 |
| 1.0 | 0.49 | ∞ | 0.25 |

Table 7 shows the simulated refractive changes (RC) due to axial crystalline lens displacement for an object at 0.4 m, 0.5 m, 0.7 m, and 4 m distance.

| Axial | RC for | RC for | RC for | RC for |
|---|---|---|---|---|
| Displacement | object at | object at | object at | object at |
| (mm) | 0.4 m (D) | 0.5 m (D) | 0.7 m (D) | 4 m (D) |
| −1.0 | −1.60 | −1.58 | −1.51 | −1.39 |
| −0.5 | −0.80 | −0.78 | −0.75 | −0.69 |
| 0 | 0 | 0 | 0 | 0 |
| 0.5 | 0.78 | 0.77 | 0.73 | 0.25 |
| 1.0 | 1.55 | 1.53 | 1.428 | 0.25 |

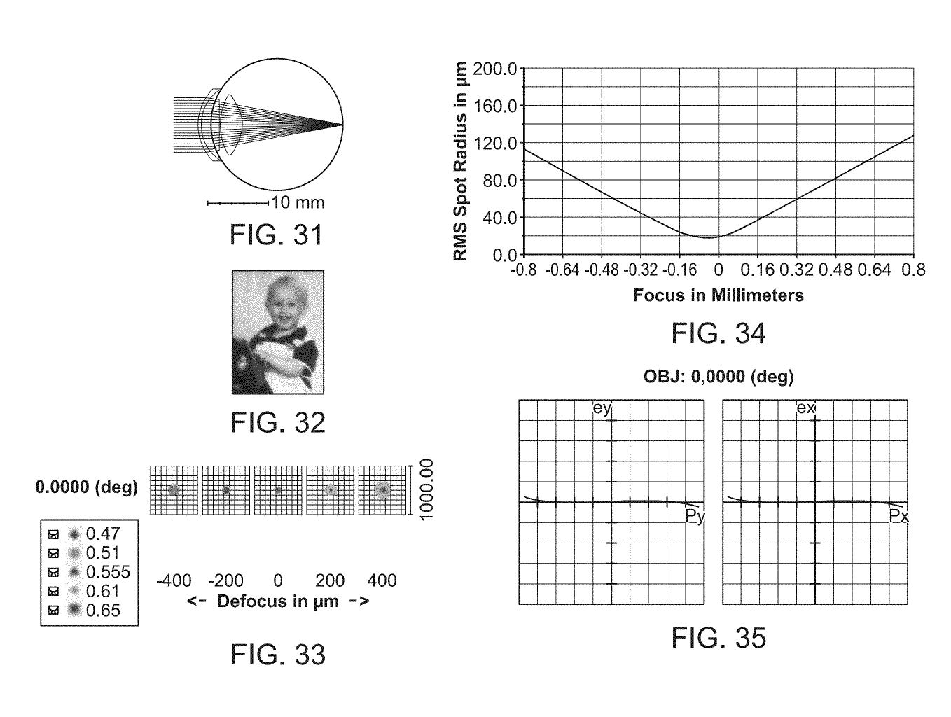

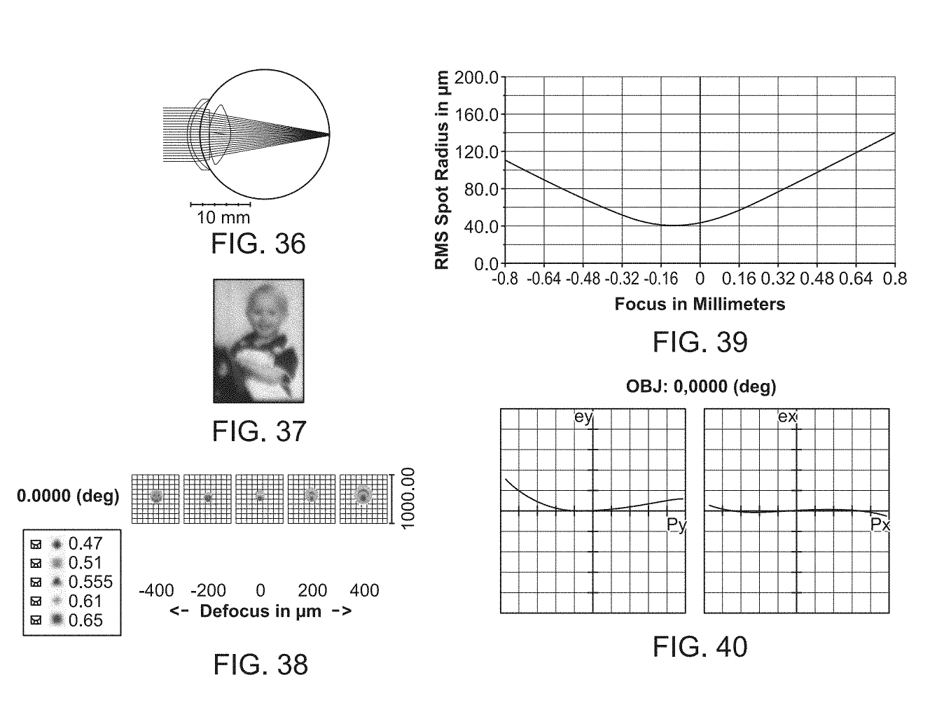

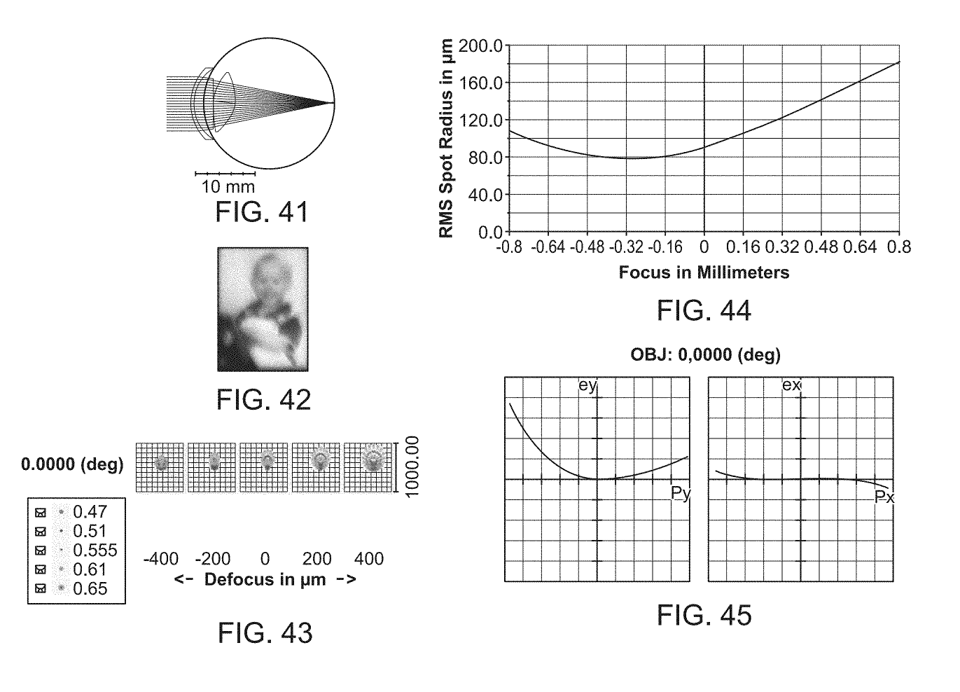

FIGS. 31-45 show simulated ocular lenses in several configurations with lens tilt. The model eye was adapted to image an object at 0.5 m distance onto the retina prior to performing the simulations. Shown here are simulations of a model eye (FIGS. 31, 36, and 41), a simulated image of a boy on the retina (FIGS. 32, 37, and 42), and the RMS value for best focal position onto the retina (FIGS. 34, 39, and 44) of each of the tree different configurations. Spot diagrams (FIGS. 33, 38, and 43) were generated at different axial positions before and after the retina to give qualitative information of coma. Additionally, Ray Fan diagrams (FIGS. 35, 40, and 45) were created showing ray coordinates relative to the chief ray, with a flat line representing an almost aberration-free beam, in order to quantitatively assess coma. The axis on the Ray Fan diagram are ±500 μm vertical and ±1 mm horizontal. P_{y} and Pₓ represent the input ray position with respect to the central ray in the x and y directions, respectively. eₓ and e_{y} represent focal error (in RMS μms) in the x and y directions, respectively. FIGS. 31-35 show a simulated ocular lens without tilt with respect to the central axis of the lens. The RMS curves showed a minimum RMS value of −0.0546, which corresponded to a spot size of 16.5 m. The maximum ray coordinate with respect to the chief ray was 27.55 μm. FIGS. 36-40 show a simulated ocular lens with 5° tilt with respect to the central axis of the lens. The simulated RMS curve showed a slight focal shift from −0.0546, the minimum RMS value simulated in FIG. 34, to −0.1056. This shift resulted in a refractive change of 0.2 D. Furthermore, the spot size increased to 36 μm, resulting in a blurred image. The effect of coma was observed in the Ray Fan diagram as well, with the maximum ray coordinate with respect to the chief ray being increased to 153 μm in the y direction. FIGS. 41-45 show a simulated ocular lens with 10° tilt with respect to the central axis of the lens. The simulated RMS curve showed a focal shift from −0.0546, the minimum RMS value simulated in FIG. 34, to −0.3108. This shift resulted in a refractive change of 0.77 D. Furthermore, the spot size increased to 76 μm, resulting in a blurred image. The effect of coma was observed in the Ray Fan diagram as well, with the maximum ray coordinate with respect to the chief ray being increased to 371 μm in the y direction. FIGS. 40 and 45 show astigmatism due to lens tilt in the y direction resulting in errors in the y direction but little change in the x direction when compared to a lens without tilt in the y direction as in FIG. 35.

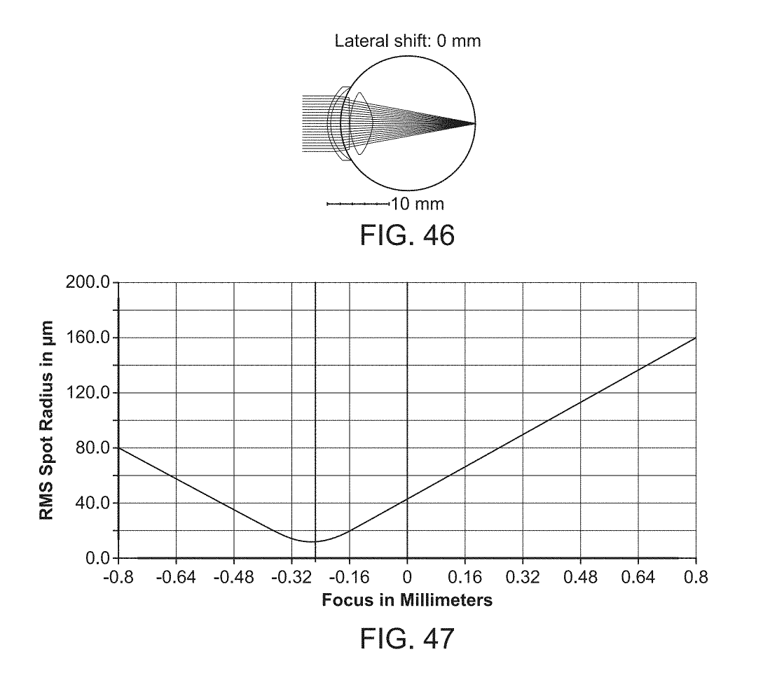

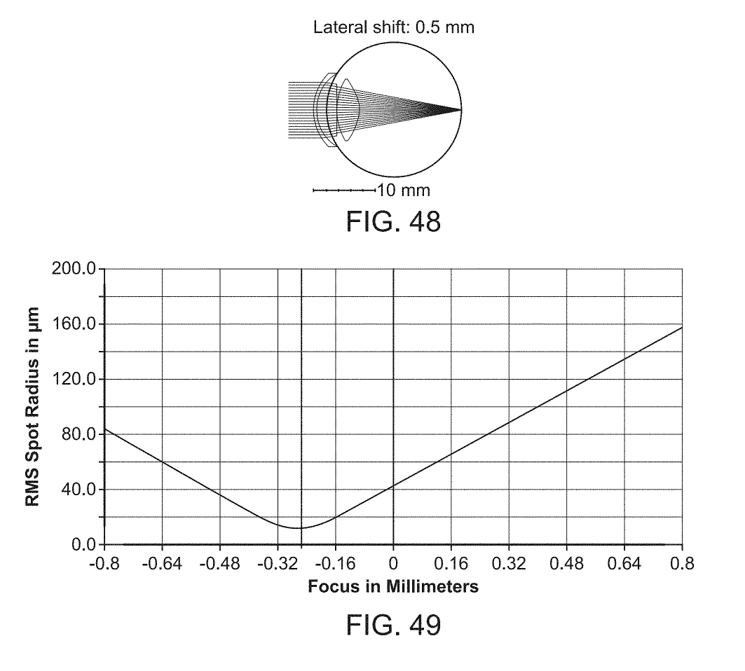

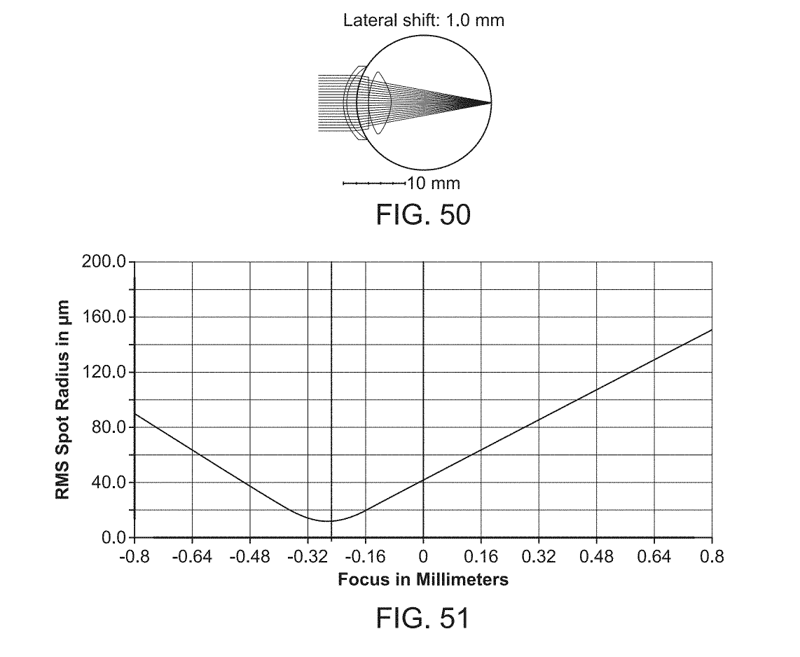

FIGS. 46-51 show a simulated ocular lens in several configurations with lateral shift of the crystalline lens. The model eye was adapted to image an object at 0.5 m distance onto the retina prior to performing the simulations. Shown here are simulations of a model eye (FIGS. 46, 48, and 50) and the RMS value for best focal position onto the retina (FIGS. 47, 49, and 51) of each of the tree different configurations. FIGS. 46-47 shows a simulated ocular lens without lateral shift of the lens. FIGS. 48-49 show a simulated ocular lens with 0.5 mm lateral displacement. The results of the simulations shown here reveal that a 0.5 mm lateral shift marginally shifts the focal position on the retina and results in a refractive power change of 0.04 D. FIGS. 50-51 show a simulated ocular lens with 1.0 mm lateral displacement. The results of the simulations shown here reveal that a 1.0 mm lateral shift marginally shifts the focal position on the retina and results in a refractive power change of 0.15 D.

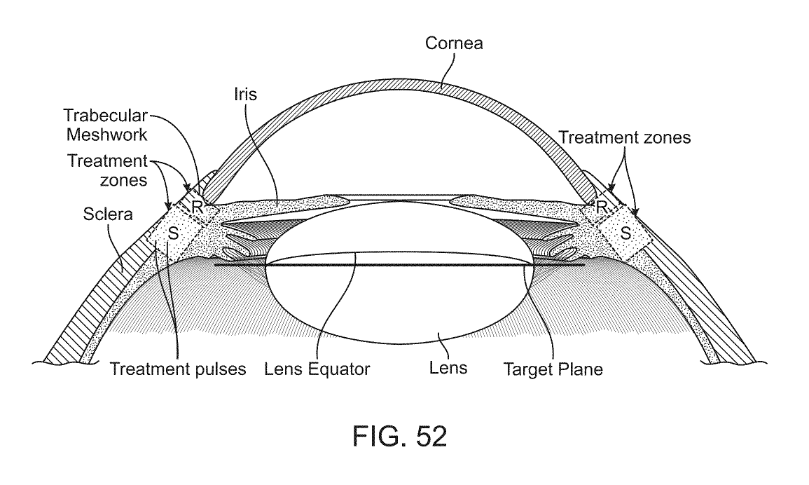

FIG. 52 shows treatment zones for ELP adjustment, for example ELA adjustment to treat coma or astigmatism. Treatment energy pulses may be focused to treatment zones, for example to a region extending from about 1 mm anterior to the anterior equatorial zonulae scleral insertion to about 1 mm posterior to the posterior equatorial zonulae scleral insertion. Alternatively or in combination, one or more treatment zones may include one or more of the sclera, the trabecular meshwork, and around Schlemm's Canal. Treatment may be for 360 degrees. Treatment may be regional, for example to induce regional tip or tilt of the lens. Treatment may be symmetric, for example the treatment zones may create a dual symmetric orthogonal bowtie pattern. As shown here, scleral shrinkage S and relaxation R may be induced symmetrically about the target plane to induce movement of the lens equator towards a line on the target plane such that the lens is rotated (e.g. tipped and/or tilted) into a target position or orientation. For example, the lens may be adjusted such that a node of the lens moves from a first position to a target location on the target plane, resulting in the lens equator aligned with the target plane. Treatment with the ELP adjustment system described herein may be used to treat RMS higher order aberrations (HOAs), for example 3^{rd} order Zernike polynomials, such as coma. RMS HOA changes may for example range from about 0 μm to about 25 μm, for example about 0 μm to about 20 μm. RMS HOA changes may for example range from about 0 μm to about 15 μm, for example about 0 μm to about 10 μm, for example about 0 μm to about 5 μm.

Although reference is made to treatment with scleral shrinkage and relaxation, the lens can be effectively moved to a desired position with scleral shrinkage without scleral relaxation, or with scleral relaxation without shrinkage. For example, the treatment may comprise stretching S as shown in FIG. 52, but without relaxation R.

The treatment energy can be delivered in many ways. The treatment pattern may comprise a plurality of generally circular scan patterns, for example. The treatment energy can be delivered with a continuous wave energy source or a pulsed energy source. Although the treatment patterns are generally circular, the treatment pattern can be configured with other shapes, such elliptical, radial or in a grid pattern over the desired treatment area.

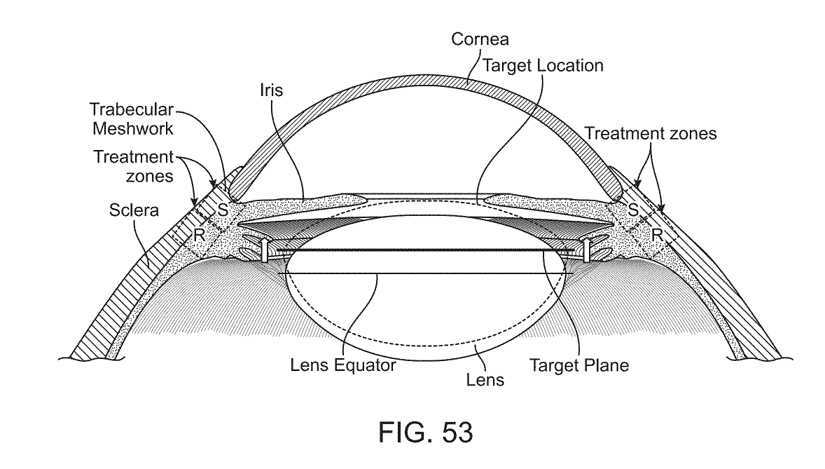

FIG. 53 shows treatment zones for ELP adjustment. The lens may be shifted from an initial position, with the lens equator lying anterior or posterior the target plane, to a target lens location. A target lens location may be determined for the patient to correct vision by the processor as previously described herein. Shown here, the lens equator lies posterior to the target plane and the target lens location therefore may be reached by myopically shifting the lens. The ELP adjustment system described herein may be used to induce scleral shrinkage S anterior to a target plane and scleral relaxation R posterior to a target plane for 360 degrees to adjust the position of the lens myopically towards the target location. Treatment pulses may be focused to treatment zones, for example to a region extending from about 1 mm anterior to the anterior equatorial zonulae scleral insertion to about 1 mm posterior to the posterior equatorial zonulae scleral insertion. Alternatively or in combination, one or more treatment zones may include one or more of the sclera, the trabecular meshwork, and around Schlemm's Canal. The lens may be myopically shifted such that a node of the lens moves from a first position to a target position along the target plane so as to laterally translate the lens (with respect to the optical axis) to the target lens location after treatment. For example, the lens may be shifted from 1.55 D to 0 D as shown here.

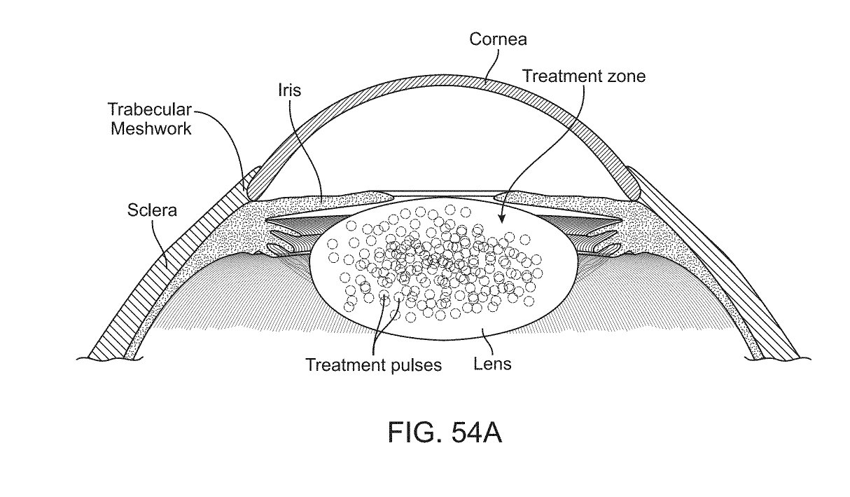

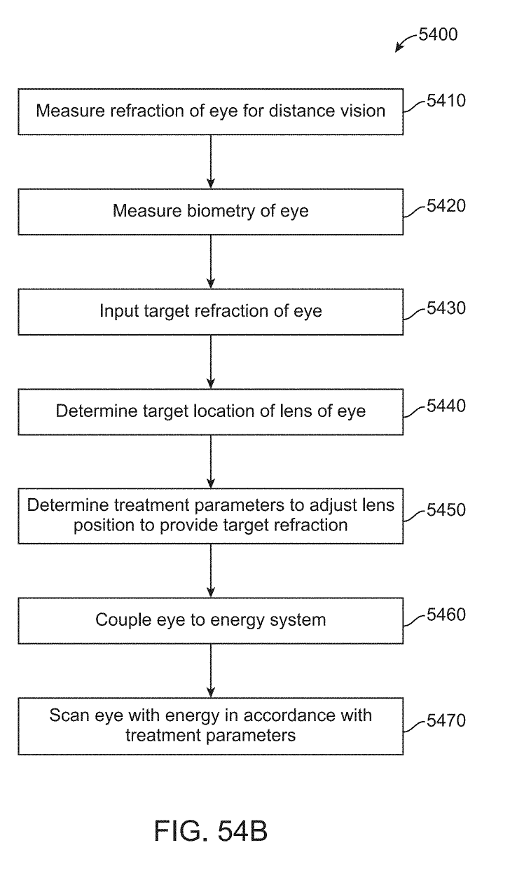

FIG. 54A shows a treatment zone for phacotripsy. The ELP adjustment system described herein may be used to mechanically induce lens softening without affecting the cornea or lens capsule, for example. Treatment pulses may be focused to a treatment zone comprising the lens cortex and nucleus. The treatment zone may be regional, for example the treatment zone may comprise one or more layers of softening at depths of the lens. Softening of the lens may be used to adjust the modulus of the lens from about 50 kPa to about 3 kPa, for example to increase accommodation. The phacotripsy treatment can be combined with ELP treatment as described herein.

FIG. 54B shows a method 5500 of ELP. At a step 5410, the refraction of eye is measured for distance vision. The refraction of the eye can be measure in many ways, for example with manifest refraction, wavefront aberrometry, or auto-refraction.

At a step 5420, biometry of eye is measured. The biometry of the eye may comprise, an axial length of the eye from the anterior corneal surface to the retina, a curvature of an anterior surface of the cornea, a curvature of the posterior surface of the cornea, a position of an anterior surface of the lens, a position of the posterior surface of the lens, a curvature of the anterior surface of the lens, a curvature of the posterior surface of the lens, the location of a node of the lens.

At a step 5430, a target refraction of eye is input into the processor system. The target refraction of the eye can depend on the age of the patient and the desired refractive outcome. For example, the target refraction of the eye may comprise emmetropia (no refractive error), or another target value, for example slightly near sighted.

At a step 5440, a target location of the lens of the eye is determined in response to the target refractive error of the eye. The target location can be determined with optical modeling. Alternatively or in combination, approximations can be used to determine the target location of the eye.

At a step 5450, treatment parameters are determined to adjust lens position to provide target refraction. The treatment parameters may comprise a laser to be used, a wavelength to be used, a power of treatment energy on to the eye, a scan rate of the laser beam on the eye, a pulse repetition rate of the laser on the eye, a type of tissue to be treated, e.g. corneal or scleral.

At a step 5460, the eye is coupled to the energy system as described herein.

At a step 5470, the eye is scanned with energy in accordance with the treatment parameters.

FIG. 54B shows a method of treating an eye in accordance with some embodiments, and a person of ordinary skill in the art will recognize many variations and adaptations. For example, some of the steps can be removed. The steps can be performed in a different order, and some of the steps can be repeated. The processor coupled to the energy delivery system can be configured to perform one or more of the steps of the method.

### EXPERIMENTAL

Table 8 describes various ELP treatment parameters that the inventors have used to induce cavitation in pig eyes for ELP adjustment with a laser energy delivery system. The sclera of each eye was treated using the methods and system disclosed herein. The wavelength of the continuous wave (CW) laser, CW power, scan speed, and beam diameter were the same for all experiments. Treatment times were varied. The eyes in Experiments 1-5 were treated in four repetitions at the sclera to induce scleral shrinkage and tensioning with either 180° arcuate patterning for cylinder (e.g. astigmatism) treatment or 360° circular (also referred to herein as annular) patterning for defocus (e.g. myopia or hyperopia) treatment. The number of repetitions refers to the number of times that the scanning patter shown in the corresponding figure was repeated. Asymmetrical scleral shrinkage, such as with an arcuate treatment pattern, may be used to correct or induce lens tilt and changes in the ELA. Symmetrical scleral shrinkage, such as with an annular treatment pattern, may be used to induce a changes in ELP by laterally shifting the lens. The eye in Experiment 6 was treated with scleral shrinkage-induced ELP adjustment followed by treatment at the cornea to induce a myopic correction.

TABLE 8
Laser treatment parameters for ELP adjustment.

| | | | | Laser | CW | Scan | Beam | | Total |
|---|---|---|---|---|---|---|---|---|---|
| | Treatment | | Treatment | wavelength | Power | Speed | diameter | | Time |
| Exp. | Site | Treatment Pattern | Type | (μm) | (W) | (mm/s) | (μm) | Reps | (s) |
| 1 | Sclera | Arcuate | Cylinder | 1.46 | 1.2 | 10 | 600 | 4 | 140 |
| | | (14 mm-20 mm, | | | | | | | |
| | | left half) | | | | | | | |
| 2 | Sclera | Arcuate | Cylinder | 1.46 | 1.2 | 10 | 600 | 4 | 140 |
| | | (14 mm-20 mm, | | | | | | | |
| | | bottom half) | | | | | | | |
| 3 | Sclera | Arcuate | Cylinder | 1.46 | 1.2 | 10 | 600 | 4 | 123 |
| | | (12 mm-18 mm, | | | | | | | |
| | | top half) | | | | | | | |
| 4 | Sclera | Circular | Defocus | 1.46 | 1.2 | 10 | 600 | 4 | 245 |
| | | (12 mm-18 mm) | | | | | | | |
| 5 | Sclera | Circular | Defocus | 1.46 | 1.2 | 10 | 600 | 4 | 290 |
| | | (15 mm-18 mm) | | | | | | | |
| 6 | Sclera + | Circular | Defocus | 1.46 | 1.2 | 10 | 600 | 8 | |
| | Cornea | | | | | | | | |

These values suggest that the lens of the eye can be moved with energy values in a range from about 50 Joules to about 500 Joules, for example with an amount of energy within a range from about 100 Joules to about 400 Joules. The amount of change of lens power can be within a range from about 1 Diopter to about 5 Diopters, for example within a range from about 1 Diopter to about 3 Diopters. The ELP treatment as disclosed herein can be used to provide an amount of energy per Diopter of lens power, the amount of energy within a range from about 50 Joules to about 300 Joules per Diopter of change in lens power.

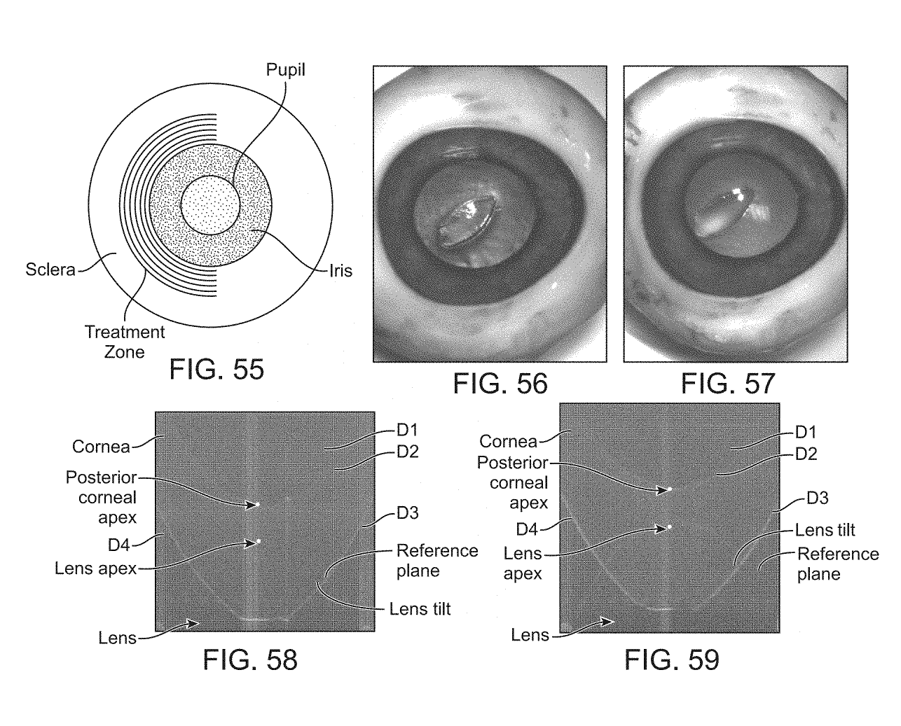

FIGS. 55-59 show the results of Experiment 1. Using the ELP adjustment parameters described in Table 8, shrinkage was induced in the sclera to induce scleral shrinkage and changes in ELP. FIG. 55 shows the treatment zone for ELP adjustment used in Experiment 1. The left half of the sclera was treated in an arcuate pattern for 180° from about 14 mm to about 20 mm from the center of the eye. FIG. 56 shows an image of the eye prior to ELP adjustment while FIG. 57 shows an image of the eye after ELP adjustment. FIG. 58 shows an OCT cross-sectional slice of the eye of FIG. 56, prior to ELP adjustment. The cornea appears upside down due to aliasing while the lens appears upright. FIG. 59 shows an OCT cross-sectional slice of the eye of FIG. 57, following ELP adjustment.

The OCT images shown were taken with an OCT system that aliased images, resulting in flipping of some of the structures, and the equations have been modified accordingly.

The anterior chamber depth (ACD) was measured at three locations before and after ELP adjustment: at the center and at the edges along the lens equator (0° and 180°). Central ACD was measured from the lens apex to the posterior corneal apex. The posterior corneal apex and the lens apex were identified prior to ELP adjustment and a line was drawn through each apex to help determine the initial distance between the apexes as shown. The distance between the posterior corneal apex and the lens apex (e.g. ACD) was defined as:

ACD=D1+D2 (3)

where D1 is the distance between the posterior corneal apex and the top of the image and D2 is the distance between the lens apex and the top of the image. The change in the central ACD was defined as

ΔACD=(D1+D2)ₚᵣₑ−(D1+D2)ₚₒₛₜ (4)

where (D1+D2)ₚᵣₑ is the ACD prior to ELP adjustment treatment and (D1+D2)ₚₒₛₜ is the ACD after ELP adjustment treatment. The ACD at 0° along the lens equator (e.g. ACD 0°) and ACD at 180° along the lens equator (e.g. ACD 180°) were measured from the lens equator to the posterior corneal apex. The lens equator at 0° was approximated by the widest visible point on the lens at 0° while the lens equator at 180° was approximated by the widest visible point on the lens at 180°, with a line between the two points representing the lens equator. The distance between the lens equator at 0° and the posterior corneal apex (e.g. ACD 0°) was defined as:

ACD0°=D1+D3 (5)

where D3 is the distance between the lens equator at 0° and the top of the image. The change in the ACD 0° was defined as

ΔACD0°=(D1+D3)ₚᵣₑ−(D1+D3)ₚₒₛₜ (6)

where (D1+D3)ₚᵣₑ is the ACD 0° prior to ELP adjustment treatment and (D1+D3)ₚₒₛₜ is the ACD 0° after ELP adjustment treatment. The distance between the lens equator at 180° and the posterior corneal apex (e.g. ACD 180°) was defined as:

ACD180°=D1+D4 (7)

where D4 is the distance between the lens equator at 180° and the top of the image. The change in the ACD 180° was defined as

ΔACD180°=(D1+D4)ₚᵣₑ−(D1+D4)ₚₒₛₜ (8)

where (D1+D4)ₚᵣₑ is the ACD 180° prior to ELP adjustment treatment and (D1+D4)ₚₒₛₜ is the ACD 180° after ELP adjustment treatment. The change in ELP in um was defined as:

$$Δ {ELP}_{μ m} = \frac{Δ ACD 180 ° + Δ ACD 0 °}{2} \qquad ( 9 )$$

where a negative change in ELP indicates movement of the lens towards the cornea. The change in ELP in Diopters was calculated assuming the linear relationship determined by the ZEMAX simulations in Table 1 above where a change in ELP of 1000 um corresponds to a change of 1.55 D. Therefore, the change in ELP in Diopters was defined as:

ΔELP_{D}=ΔELP_{μm}=0.00155 (10)

Although the ZEMAX values are shown for a model eye, specific parameters of an individual eye can be used to determine the amount of lens movement to correct vision with ELP. For example, the axial length, nodal position of the lens, corneal curvature and other parameters can be entered in order to determine the amount of lens movement to correct vision and the treatment customized for a specific patient.

The initial lens tilt was calculated with respect to an arbitrary reference plane. The post-operative lens tilt was calculated with respect to the reference plane and the change in lens tilt was defined as the difference between the initial lens tilt and the post-operative lens tilt. A negative change in lens tilt or angle indicates tilting of the lens away from the cornea.

The changes in ELP, lens tilt, and ACD for Experiment 1 are listed in Table 9 below. 180° treatment of the sclera resulted in a change in ELP of −100 um, which is equivalent to −0.15 D, and lens tilt of 5°. The changes in ACD at 0°, 180°, and the center were −176 um, 29 um, and 628 um, respectively.

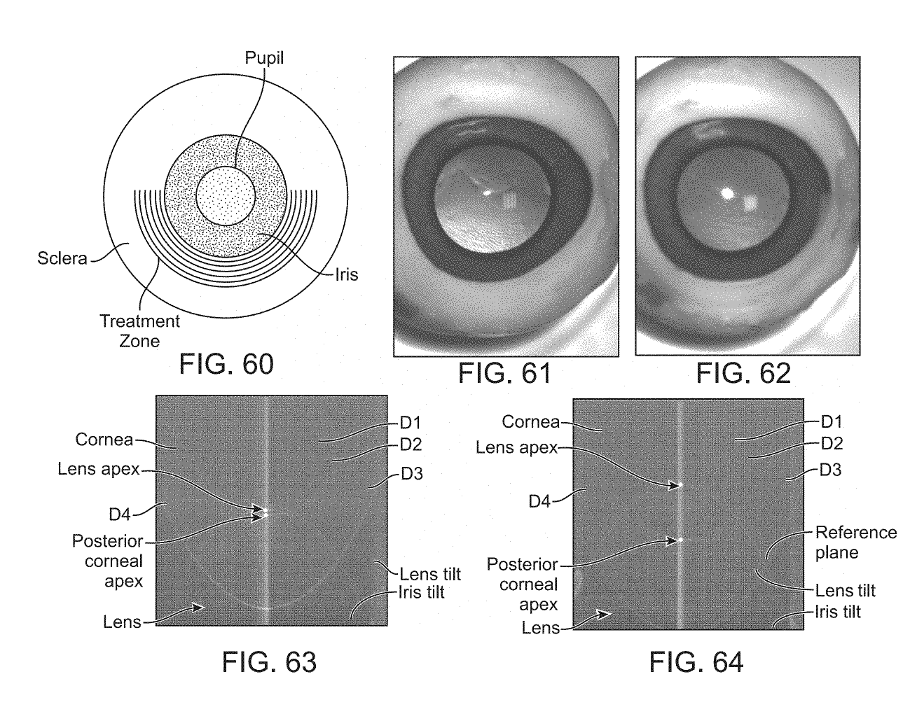

FIGS. 60-64 show the results of Experiment 2. Using the ELP adjustment parameters described in Table 8, non-thermal cavitation was induced in the sclera to induce scleral shrinkage and changes in ELP. FIG. 60 shows the treatment zone for ELP adjustment used in Experiment 2. The bottom half of the sclera was treated in an arcuate pattern for 180° from about 14 mm to about 20 mm from the center of the eye. FIG. 61 shows an image of the eye prior to ELP adjustment while FIG. 62 shows an image of the eye after ELP adjustment. FIG. 63 shows an OCT cross-sectional slice of the eye of FIG. 61, prior to ELP adjustment. The cornea appears upside down due to aliasing while the lens appears upright. FIG. 64 shows an OCT cross-sectional slice of the eye of FIG. 62, following ELP adjustment.

The anterior chamber depth (ACD) was measured at three locations before and after ELP adjustment: at the center and at the edges along the lens equator (0° and 180°). Central ACD was measured from the lens apex to the posterior corneal apex. The posterior corneal apex and the lens apex were identified prior to ELP adjustment and a line was drawn through each apex to help determine the initial distance between the apexes as shown. The distance between the posterior corneal apex and the lens apex (e.g. ACD) was calculated using Formula 3 as previously described. The change in the central ACD was calculated using Formula 4 as previously described. The ACD at 0° along the lens equator (e.g. ACD 0°) and ACD at 180° along the lens equator (e.g. ACD 180°) were measured from the lens equator to the posterior corneal apex. The lens equator at 0° was approximated by the widest visible point on the lens at 0° while the lens equator at 180° was approximated by the widest visible point on the lens at 180°, with a line between the two points representing the lens equator. The distance between the lens equator at 0° and the posterior corneal apex (e.g. ACD 0°) was calculated using Formula 5 as previously described. The change in the ACD 0° was calculated using Formula 6 as previously described. The distance between the lens equator at 180° and the posterior corneal apex (e.g. ACD 180°) was calculated using Formula 7 as previously described. The change in the ACD 180° was calculated using Formula 8 as previously described. The change in ELP in um was calculated using Formula 9 as previously described. The change in ELP in Diopters was calculated using Formula 10 as previously described. A negative change in ELP indicates movement of the lens towards the cornea.

The initial lens tilt was calculated with respect to an arbitrary reference plane. The post-operative lens tilt was calculated with respect to the reference plane and the change in lens tilt was defined as the difference between the initial lens tilt and the post-operative lens tilt. A negative change in lens tilt or angle indicates tilting of the lens away from the cornea. The initial iris tilt was calculated as the angle of a line drawn from one visible edge of the iris to another visible edge of the iris prior to treatment with respect to an arbitrary reference plane. The post-operative iris tilt was calculated and the change in iris tilt was defined as the difference between the initial iris tilt and the post-operative iris tilt.

The changes measured in ELP, lens tilt, and ACD for Experiment 2 are listed in Table 9 below. 180° treatment of the scleral resulted in a change in ELP of −954 um, which is equivalent to −1.48 D, and lens tilt of −11°. The changes in ACD at 0°, 180°, and the center were −834 um, −1186 um, and 558 um, respectively.

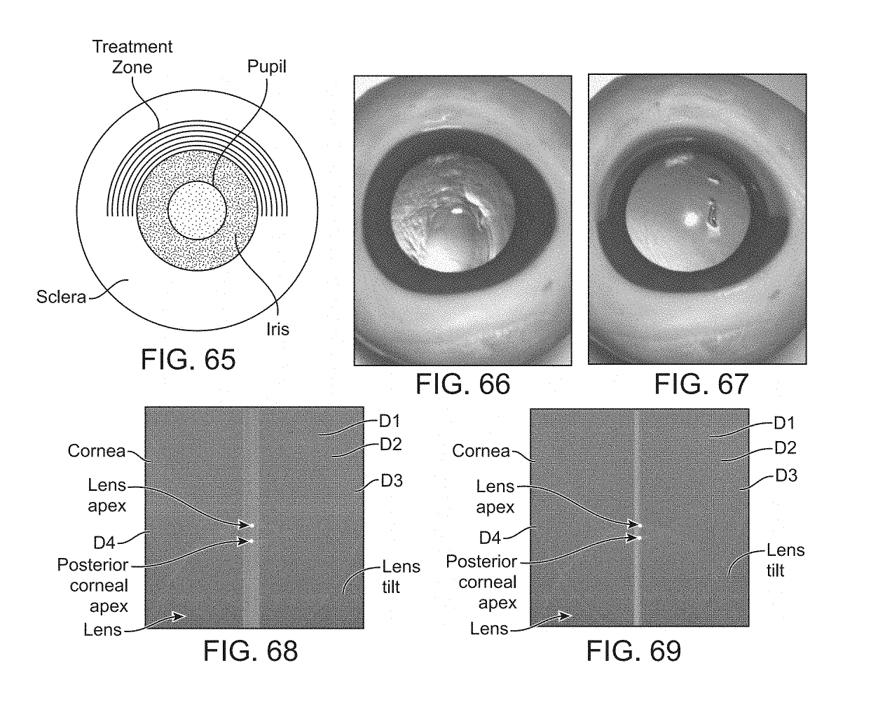

FIGS. 65-69 show the results of Experiment 3. Using the ELP adjustment parameters described in Table 8, non-thermal cavitation was induced in the sclera to induce scleral shrinkage and changes in ELP. FIG. 65 shows the treatment zone for ELP adjustment used in Experiment 3. The top half of the sclera was treated in an arcuate pattern for 180° from about 12 mm to about 18 mm from the center of the eye. FIG. 66 shows an image of the eye prior to ELP adjustment while FIG. 67 shows an image of the eye after ELP adjustment. FIG. 68 shows an OCT cross-sectional slice of the eye of FIG. 66, prior to ELP adjustment. The cornea appears upside down due to aliasing while the lens appears upright. FIG. 69 shows an OCT cross-sectional slice of the eye of FIG. 67, following ELP adjustment.

The anterior chamber depth (ACD) was measured at three locations before and after ELP adjustment: at the center and at the edges along the lens equator (0° and 180°). Central ACD was measured from the lens apex to the posterior corneal apex. The posterior corneal apex and the lens apex were identified prior to ELP adjustment and a line was drawn through each apex to help determine the initial distance between the apexes as shown. The distance between the posterior corneal apex and the lens apex (e.g. ACD) was calculated using Formula 3 as previously described. The change in the central ACD was calculated using Formula 4 as previously described. The ACD at 0° along the lens equator (e.g. ACD 0°) and ACD at 180° along the lens equator (e.g. ACD 180°) were measured from the lens equator to the posterior corneal apex. The lens equator at 0° was approximated by the widest visible point on the lens at 0° while the lens equator at 180° was approximated by the widest visible point on the lens at 180°, with a line between the two points representing the lens equator. The distance between the lens equator at 0° and the posterior corneal apex (e.g. ACD 0°) was calculated using Formula 5 as previously described. The change in the ACD 0° was calculated using Formula 6 as previously described. The distance between the lens equator at 180° and the posterior corneal apex (e.g. ACD 180°) was calculated using Formula 7 as previously described. The change in the ACD 180° was calculated using Formula 8 as previously described. The change in ELP in um was calculated using Formula 9 as previously described. The change in ELP in Diopters was calculated using Formula 10 as previously described. A negative change in ELP indicates movement of the lens towards the cornea.

The initial lens tilt was calculated with respect to an arbitrary reference plane. The post-operative lens tilt was calculated with respect to the reference plane and the change in lens tilt was defined as the difference between the initial lens tilt and the post-operative lens tilt. A negative change in lens tilt or angle indicates tilting of the lens away from the cornea.

The changes measured in ELP, lens tilt, and ACD for Experiment 3 are listed in Table 9 below. 180° treatment of the scleral resulted in a change in ELP of 637 um, which is equivalent to 0.99 D, and lens tilt of −3°. The changes in ACD at 0°, 180°, and the center were 558 um, 705 um, and 681 um, respectively.

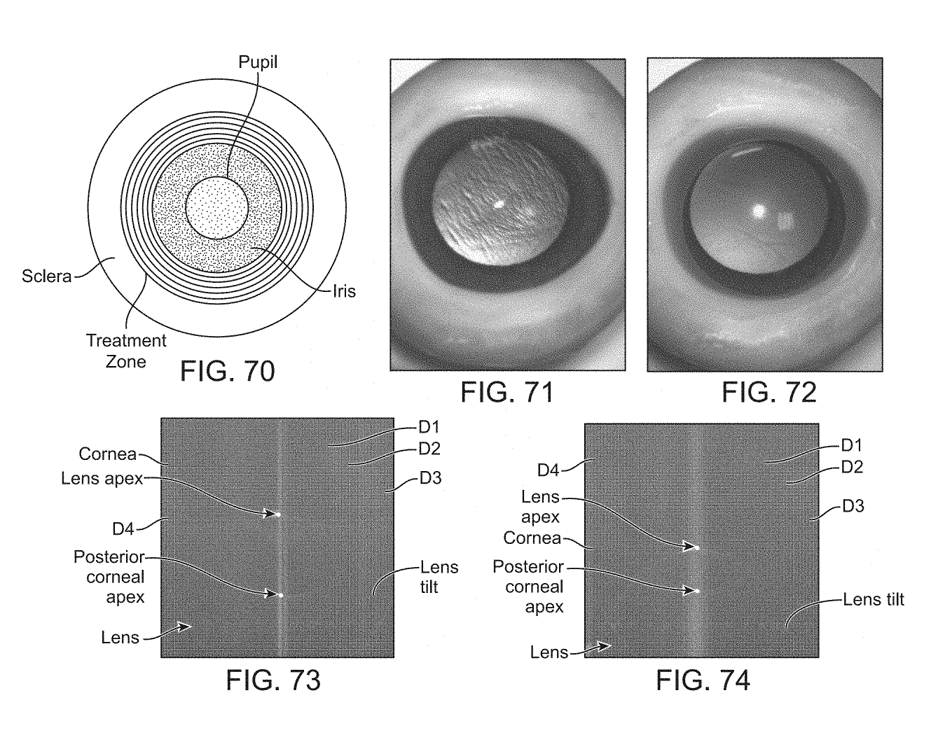

FIGS. 70-74 show the results of Experiment 4. Using the ELP adjustment parameters described in Table 8, non-thermal cavitation was induced in the sclera to induce scleral shrinkage and changes in ELP. FIG. 70 shows the treatment zone for ELP adjustment used in Experiment 4. The sclera was treated in an annular (also referred to herein as circular) pattern for 360° from about 12 mm to about 18 mm from the center of the eye. FIG. 71 shows an image of the eye prior to ELP adjustment while FIG. 72 shows an image of the eye after ELP adjustment. FIG. 73 shows an OCT cross-sectional slice of the eye of FIG. 71, prior to ELP adjustment. The cornea appears upside down due to aliasing while the lens appears upright. FIG. 74 shows an OCT cross-sectional slice of the eye of FIG. 72, following ELP adjustment.

The anterior chamber depth (ACD) was measured at three locations before and after ELP adjustment: at the center and at the edges along the lens equator (0° and 180°). Central ACD was measured from the lens apex to the posterior corneal apex. The posterior corneal apex and the lens apex were identified prior to ELP adjustment and a line was drawn through each apex to help determine the initial distance between the apexes as shown. The distance between the posterior corneal apex and the lens apex (e.g. ACD) was calculated using Formula 3 as previously described. The change in the central ACD was calculated using Formula 4 as previously described. The ACD at 0° along the lens equator (e.g. ACD 0°) and ACD at 180° along the lens equator (e.g. ACD 180°) were measured from the lens equator to the posterior corneal apex. The lens equator at 0° was approximated by the widest visible point on the lens at 0° while the lens equator at 180° was approximated by the widest visible point on the lens at 180°, with a line between the two points representing the lens equator. The distance between the lens equator at 0° and the posterior corneal apex (e.g. ACD 0°) was calculated using Formula 5 as previously described. The change in the ACD 0° was calculated using Formula 6 as previously described. The distance between the lens equator at 180° and the posterior corneal apex (e.g. ACD 180°) was calculated using Formula 7 as previously described. The change in the ACD 180° was calculated using Formula 8 as previously described. The change in ELP in um was calculated using Formula 9 as previously described. The change in ELP in Diopters was calculated using Formula 10 as previously described. A negative change in ELP indicates movement of the lens towards the cornea.

The initial lens tilt was calculated with respect to an arbitrary reference plane. The post-operative lens tilt was calculated with respect to the reference plane and the change in lens tilt was defined as the difference between the initial lens tilt and the post-operative lens tilt. A negative change in lens tilt or angle indicates tilting of the lens away from the cornea.

The changes measured in ELP, lens tilt, and ACD for Experiment 4 are listed in Table 9 below. 180° treatment of the scleral resulted in a change in ELP of 399 um, which is equivalent to 0.62 D, and lens tilt of 1°. The changes in ACD at 0°, 180°, and the center were 528 um, 223 um, and 1245 um, respectively.

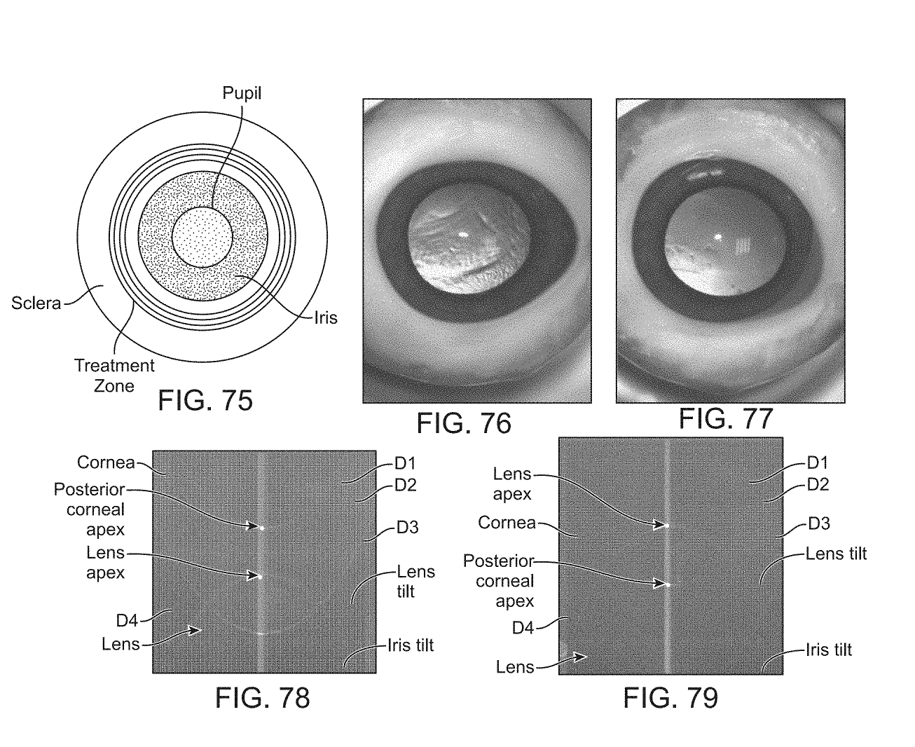

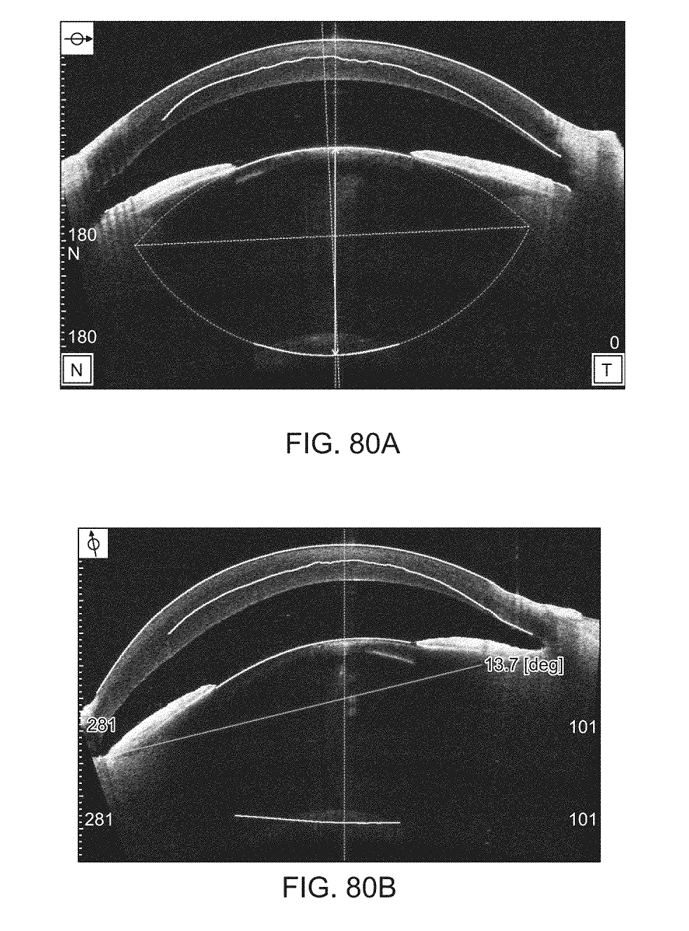

FIGS. 75-79 show the results of Experiment 5. Using the ELP adjustment parameters described in Table 8, non-thermal cavitation was induced in the sclera to induce scleral relaxation and changes in ELP. FIG. 75 shows the treatment zone for ELP adjustment used in Experiment 5. The sclera was treated in a circular pattern for 360° from about 15 mm to about 18 mm from the center of the eye. FIG. 76 shows an image of the eye prior to ELP adjustment while FIG. 77 shows an image of the eye after ELP adjustment. FIG. 78 shows an OCT cross-sectional slice of the eye of FIG. 76, prior to ELP adjustment. The cornea appears upside down due to aliasing while the lens appears upright. FIG. 79 shows an OCT cross-sectional slice of the eye of FIG. 77, following ELP adjustment.

The anterior chamber depth (ACD) was measured at three locations before and after ELP adjustment: at the center and at the edges along the lens equator (0° and 180°). Central ACD was measured from the lens apex to the posterior corneal apex. The posterior corneal apex and the lens apex were identified prior to ELP adjustment and a line was drawn through each apex to help determine the initial distance between the apexes as shown. The distance between the posterior corneal apex and the lens apex (e.g. ACD) was calculated using Formula 3 as previously described. The change in the central ACD was calculated using Formula 4 as previously described. The ACD at 0° along the lens equator (e.g. ACD 0°) and ACD at 180° along the lens equator (e.g. ACD 180°) were measured from the lens equator to the posterior corneal apex. The lens equator at 0° was approximated by the widest visible point on the lens at 0° while the lens equator at 180° was approximated by the widest visible point on the lens at 180°, with a line between the two points representing the lens equator. The distance between the lens equator at 0° and the posterior corneal apex (e.g. ACD 0°) was calculated using Formula 5 as previously described. The change in the ACD 0° was calculated using Formula 6 as previously described. The distance between the lens equator at 180° and the posterior corneal apex (e.g. ACD 180°) was calculated using Formula 7 as previously described. The change in the ACD 180° was calculated using Formula 8 as previously described. The change in ELP in um was calculated using Formula 9 as previously described. The change in ELP in Diopters was calculated using Formula 10 as previously described. A negative change in ELP indicates movement of the lens towards the cornea.

The initial lens tilt was calculated with respect to an arbitrary reference plane. The post-operative lens tilt was calculated with respect to the reference plane and the change in lens tilt was defined as the difference between the initial lens tilt and the post-operative lens tilt. A negative change in lens tilt or angle indicates tilting of the lens away from the cornea. The initial iris tilt was calculated as the angle of a line drawn from one visible edge of the iris to another visible edge of the iris prior to treatment with respect to an arbitrary reference plane. The post-operative iris tilt was calculated and the change in iris tilt was defined as the difference between the initial iris tilt and the post-operative iris tilt.

The changes measured in ELP, lens tilt, and ACD for Experiment 5 are listed in Table 9 below. 180° treatment of the sclera resulted in a change in ELP of −728 um, which is equivalent to −1.13 D, and lens tilt of −1°. The changes in ACD at 0°, 180°, and the center were −622 um, −857 um, and 640 um, respectively.

The results of Experiment 6 are shown in Table 9. An eye was treated with scleral ELP adjustment followed by treatment at the cornea to induce a myopic correction. As shown, the change in ELP was measured to be −5.5 D while the change in ACD was −3399 um at the center.

TABLE 9
Results of ELP adjustment Experiments 1-6.

| | Δ 0° | Δ 180° | | Δ Lens | | |
|---|---|---|---|---|---|---|
| Exper- | ACD | ACD | Δ ACD | tilt | Δ ELP | Δ ELP |
| iment | (um) | (um) | (um) | (°) | (um) | (Diopters) |
| 1 | −176 | 29 | 628 | 5 | −100 | −0.15 |
| 2 | −834 | −1186 | 558 | −11 | −954 | −1.48 |
| 3 | 558 | 705 | 681 | −3 | 637 | 0.99 |
| 4 | 528 | 223 | 1245 | 1 | 399 | 0.62 |
| 5 | −622 | −857 | 640 | −1 | −728 | −1.13 |
| 6 | NA | NA | −3399 | NA | 0 | −5.5 |

Further changes were observed in the surface regularity of the lens capsule, the circularity of the iris, and the anterior lens curvature.

A visual comparison of the pre-operative images (FIGS. 56, 61, 66, 71, and 76) with their respective post-operative images (FIGS. 57, 62, 67, 72, and 77, respectively) shows that treatment of the sclera with the ELP adjustment system described herein may lead to a reduction in or smoothing of lens capsular wrinkles, resulting in an increase in capsular surface regularity which may lead to better optical quality.

Analysis of the pre-operative images (FIGS. 56, 61, 66, 71, and 76) with their respective post-operative images (FIGS. 57, 62, 67, 72, and 77, respectively) shows that treatment of the sclera with the ELP adjustment system described herein may lead to improvements in iris circularity. Iris circularity may be defined as a ratio between the length of the major axis and the length of the minor axis of the iris, with a ratio of 1 being a circular iris. The post-operative eye treated in Experiments 1-5 had more circular irises, with about 5% improvement in circularity, when compared to the initial pre-operative shape of their respective irises.

Analysis of pre-operative OCT cross-sectional images (FIGS. 58, 63, 68, 73, and 78) with their respective post-operative OCT images (FIGS. 59, 64, 69, 74, and 79, respectively) shows that treatment of the sclera with the ELP adjustment system described herein may lead to improvements in anterior lens curvature as well as ELP. Changes in ELP adjustment can be used to compensate for changes in the anterior lens curvature. For example, adjustment of ELP in the negative direction (towards the cornea) results in a myopic change in the refraction of the eye. As the anterior lens curvature flattens away from the cornea, the eye becomes hyperopic. Therefore, a myopic ELP adjustment may be used to adjust the curvature of the lens of the eye or the position of the lens of the eye and combinations thereof to provide a desired refraction of the patient.

FIGS. 80A and 80B show OCT cross-sectional slices of an eye treated in accordance with treatment parameters as disclosed herein. The eye was treated with a 270 degree treatment, 400 mW of 2.1 um laser energy, a scan rate of 10 mm/sec, in circular scan patterns from 12 mm to 15 mm in 0.5 mm increments (7 circular scans), and this pattern was repeated four times. The eye was scanned with a commercially available OCT system after laser treatment. The eye was aligned with the OCT system subsequent to treatment such that the treated area extended from approximately −45 degrees to approximately 225 degrees, with the untreated region extending from approximately 225 degrees to 315 degrees. FIG. 80A shows the OCT scan from approximately 0 to 180 degrees, and FIG. 80B shows the OCT scan from approximately 260 degrees to approximately 80 degrees. FIG. 80A shows a scan along the eye through treated annular regions, and the lens of the eye shows relatively little tilt. FIG. 80B shows a scan along the eye encompassing the treated and untreated regions, and this scan shows significantly greater tilt of approximately 13 degrees. The shrinkage of scleral tissue as described herein appears to have induced shrinking of the scleral tissue anterior to the lens and pulled the lens anteriorly, so as to induce the tilt. The shrinkage of scleral tissue would be expected to symmetrically pull the lens forward along the 0 to 180 meridian, such that the induced tilt is much less visible along this meridian Similar experiments can be conducted to show movement of the lens without substantial tilt in order to correct spherical errors of the eye as described herein.

Although the above experiments were conducted on ex vivo porcine eyes, a person of ordinary skill in the art can conduct in vivo experiments on living human eyes in order to develop nomograms to position the lens of the eye to correct vision with treatment energies as described herein. For example, the laser system can be coupled to an OCT measurement systems as described herein, and OCT measurements on living human eyes during surgery can be used to measure movement of the lens in response to treatment as described herein, in order to determine amounts and locations of energy treatment that produce desired amounts of movement of the lens. The OCT system can be coupled to the laser system with a beam splitter, such that the laser beam path and OCT measurement beam path are aligned, and the patient can be measured in situ.

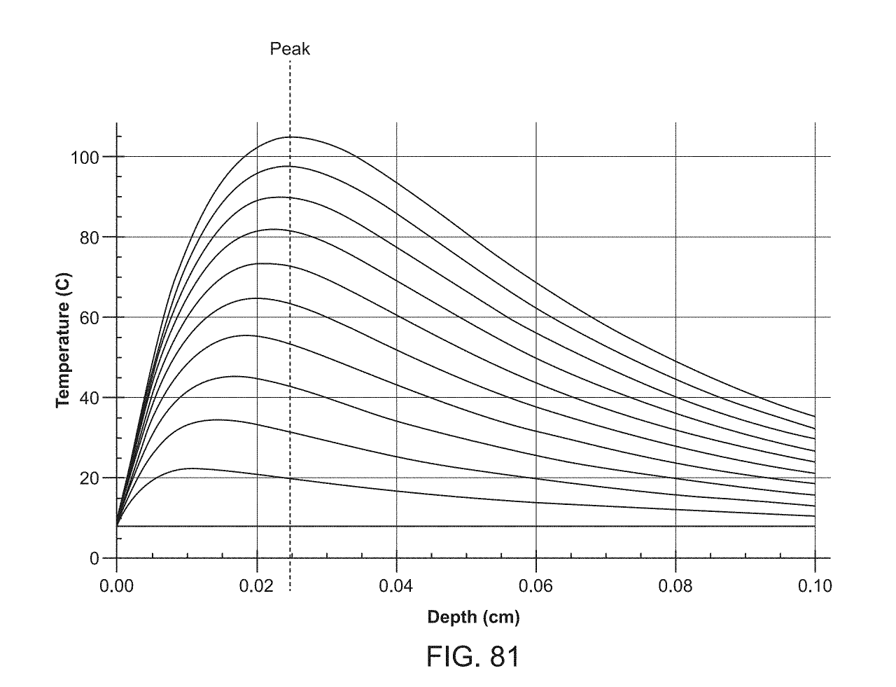

FIG. 81 shows temperature profiles of an eye treated with a laser beam with the eye coupled to a chilled lens based on computer modeling. FIG. 81 shows a plot of a finite element analysis model of temperature (in degrees C.) as a function of scleral tissue depth (in mm). Temperature profiles of an eye treated with a 2.1 um laser beam were simulated with the eye coupled to a chilled lens using computer modeling. The finite element analysis assumed anisotropy in the radial and axial dimensions. Each line represents a different amount of energy deposited within the tissue. Changes in the simulated power and/or time were used to alter the total simulated energy deposited within the tissue. For each of the treatment parameters simulated, the tissue reached a peak temperature at about 250 um deep. The simulation was conducted assuming a laser intensity of 200 W cm⁻² and tissue absorptivity at 2.1 um of 20 mm⁻¹.

The chilled lens was modeled at the surface with a fixed temperature of about 6 to 8 C, assuming that the lens would remain at the substantially the same temperature to which it was cooled prior to or during (via active cooling methods) treatment. Such temperatures may for example be reached by placing a cooled lens on the surface of the eye for a predetermined amount of time, for example about 10 to about 30 seconds, prior to initiating treatment as described herein. The amount of time sufficient to cool and protect the surface of the eye may vary depending on the thickness of the cornea and the extent of suction applied by the system to maintain contact between the lens, patient interface, and the surface of the eye.

In some instances, a temperature of about 60 to about 70 degrees may be reached at a depth of about 50 um to about 400 um in the sclera. A temperature of about 60 to about 70 degrees may be reached at a depth of about 50 um to about 300 um in the cornea. The conjunctiva and/or the epithelium may remain substantially intact during heating of the underlying tissue due to the use of a cooling element or heat sink, such as a chilled contact lens, to inhibit damage to these sensitive surface tissues of the eye. The surface of the sclera may be chilled so as to remain at or near 4 C.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An apparatus to adjust one or more of an effective position or an orientation of a lens of the eye, the apparatus comprising:
an energy source configured to deliver energy to the eye to one or more of shrink or relax a sclera of the eye in order to adjust the one or more of the effective position or orientation of the lens of the eye.

2. The apparatus of claim 1, wherein the energy source is configured to move the lens anteriorly to treat farsightedness, to move the lens posteriorly to treat nearsightedness, or to tilt the lens to treat astigmatism, and optionally combinations thereof.

3. The apparatus of claim 1, further comprising:
a processor configured with instructions to determine the effective position or orientation of the lens of the eye and to determine a direction of movement or rotation of the lens toward the effective lens position or orientation and optionally wherein the direction of movement or rotation is selected from the group consisting of posterior movement to treat myopia, anterior movement to treat hyperopia and rotation to treat astigmatism.

4. The apparatus of claim 3 wherein the processor is coupled to the energy source and configured to heat sclera of the eye to a temperature within a range from about 50 C to about 70 C for an amount of time sufficient to shrink the sclera of the eye in order to move the lens of the eye toward the effective lens position and optionally wherein the processor is configured with instructions to heat the sclera of the eye to a temperature within a range from about 50 C to about 80 C for an amount of time sufficient to relax the sclera of the eye in order to move the lens toward the effective lens position.

5. The apparatus of claim 3 wherein the processor is configured to determine a scanning pattern of energy to be delivered to the eye to move the lens toward the effective lens position.

6. The apparatus of claim 3 wherein the processor is coupled to the energy source and configured to deliver shrinkage energy to a first region of the sclera to shrink the sclera and relaxation energy to a second region of the sclera to relax the sclera in response to the direction of movement or rotation of the lens.

7. The apparatus of claim 6, wherein a target lens position corresponds to a target plane of the lens, and wherein the shrinkage region and the relaxation region are arranged on opposite sides of the target plane.

8. The apparatus of claim 7, wherein the relaxation region of the sclera corresponds to a location of an equator of the lens prior to treatment and the lens moves away from the relaxation region toward the stiffened region with treatment.

9. The apparatus of claim 8, wherein a target orientation of the lens corresponds to a target plane of the lens, and wherein the shrinkage region and the relaxation region are arranged on the same side of the target plane and on opposite sides of the eye in order to urge the lens toward the target orientation.

10. The apparatus of claim 9, wherein the lens rotates away from the relaxation region on a first side of the eye and toward the shrinkage region on the second side of the eye.

11. The apparatus of claim 3, wherein the processor is configured to receive a refraction of the eye and to determine an amount of movement of the lens of the eye to adjust the refraction of the eye to a target refraction of the eye and wherein movement of the lens anteriorly adjusts the refraction of the eye negatively and movement of lens posteriorly adjusts the refraction of the eye positively.

12. The apparatus of claim 11, wherein the processor is configured to determine a scanning pattern of energy to be delivered to the eye to move the lens to adjust the refraction to the target amount.

13. The apparatus of claim 11, wherein the processor is configured to adjust the refraction of the lens at a rate within a range from about 0.75 D to about 1.75 D per mm of movement of the lens.

14. The apparatus of claim 11, wherein the processor is configured to receive an axial length of the eye to determine the amount of movement.

15. The apparatus of claim 1, wherein a target lens position corresponds to a target plane of the lens, and wherein the shrinkage region and the relaxation region are arranged on opposite sides of the target plane to move the lens away from the relaxation region and toward the shrinkage region.

16. The apparatus of claim 1, further comprising:
a delivery system configured to deliver the energy wherein the delivery system is configured to deliver the energy to one or more of a relaxation region of the sclera or a shrinkage region of the sclera, in order to move the lens anteriorly to treat farsightedness, posteriorly to treat nearsightedness, or to tilt the lens to treat astigmatism, and optionally combinations thereof.

17. The apparatus of claim 16, wherein the delivery system is configured to deliver an annular pattern of shrinkage energy anterior to the lens equator in order to move the lens anteriorly.

18. The apparatus of claim 16, wherein the delivery system is configured to deliver an annular pattern of shrinkage energy posterior to the lens equator in order to move the lens posteriorly

19. The apparatus of claim 16, wherein the delivery system is configured to deliver an annular pattern of relaxation energy anterior to the lens equator in order to move the lens posteriorly.

20. The apparatus of claim 16, wherein the delivery system is configured to deliver an annular pattern of relaxation energy posterior to the lens equator in order to move the lens anteriorly.

21. The apparatus of claim 1, wherein one or more of the energy source, the processor or the energy delivery system is configured to deliver energy to a scleral shrinkage region posterior to the lens equator in order to urge the lens posteriorly to treat myopia.

22.-52. (canceled)
